(19) United States
(12) Patent Application Publication
(10) Pub. No.: US 2025/0085273 A1
(43) Pub. Date: Mar. 13, 2025

(54) NOVEL RAPID DRUG DISCOVERY FOR SEPSIS AND AGENTS FOR USE AS SEPSIS THERAPY

(71) Applicant: THE GOVERNMENT OF THE UNITED STATES, AS REPRESENTED BY THE SECRETARY OF THE ARMY, Fort Detrick, MD (US)

(72) Inventors: Mark W. WIDDER, Waynesboro, PA (US); Chance M. CARBAUGH, McConnellsburg, PA (US); William H. VAN DER SCHALIE, Fort Detrick, MD (US); Charlotte A. LANTERI, Boyds, MD (US); Kevin S. AKERS, San Antonio, TX (US)

(21) Appl. No.: 18/800,672

(22) Filed: Aug. 12, 2024

Related U.S. Application Data
(60) Provisional application No. 63/532,142, filed on Aug. 11, 2023.

(51) Int. Cl. G01N 33/50

(52) U.S. Cl. CPC G01N 33/5088

(57) ABSTRACT

In this disclosure, a zebrafish model is established for rapid screening of potential drug candidates for prevention and treatment of sepsis. To this end, three endpoints (mortality, tail-fin edema, and reactive oxygen species detection) screening method is established to identify drugs of potential use among approved drugs and/or novel small molecules, and 16 drugs were identified, which target serotonin receptors, receptor tyrosine kinases, dietary supplements, antihistamine drugs, serotonin uptake inhibitors, monoamine oxidase inhibitors, and other antipsychotic drugs.

### GOVERNMENT INTEREST

The invention described herein may be manufactured, used and licensed by or for the United States Government.

### BACKGROUND

### 1. Field of the Invention

The invention relates to the general field of septic shock treatment, and in particular to a potential sepsis therapeutic drugs identified using a zebrafish screening model and use thereof.

### 2. Background

The advances in care for the warfighter including body armor, medical/surgical care on and off the battlefield, and the rapid evacuation from the battlefield, have yielded a greater number of warfighters who survive injuries sustained on the battlefield, but unfortunately many of them succumb to complications from their wounds later. In particular, the high energy wounds sustained from gunshots, IEDs, and shrapnel that cause fractures are the most common in recent conflicts and are highly susceptible to bacterial infection including osteomyelitis (Yun et al. 2006; Petersen et al. 2007). These combat injuries account for approximately 65% of the total percentage of injuries and are evenly distributed between upper and lower extremities. In some cases, wound infections can lead to sepsis, which has a significant effect on increasing morbidity and mortality among military casualties.

In Vietnam, sepsis was the third leading cause of death among those admitted to hospitals, and in Operation Iraqi Freedom and Operation Enduring Freedom, sepsis was found to be the fourth leading cause of potential survivable injuries behind central nervous system injury, hemorrhage, and airway disorders (Kelly et al., 2008).

In addition, sepsis-related mortality is increasing, in part due to antibiotic resistance and increased bacterial virulence. The emergence of multidrug resistant bacteria has become a global problem for current antibiotic therapies, thus becoming a difficult challenge for hospitals worldwide, and it is a threat to effective treatment of infections among combat-wounded Service Members. Combat wound infections can progress rapidly to life-threatening sepsis with a median time of three days from injury to the onset of sepsis (Weintrob et al., 2018). A total of 5,278 sepsis hospitalizations were recorded in the active component of the U.S. military from 2011 to 2020 (Snitchler et al., 2021). A recent review of the Department of Defense Trauma Registry from 2007 to 2020 found that patients with combat injury related wound infections who were diagnosed with sepsis had significantly higher mortality rates than non-septic patients (21.9% vs. 4.3%, p<0.001) (Carius et al., 2021). The incidence of infection and sepsis is expected to increase in future conflicts due to multi-domain operations that will result in prolonged field care conditions.

Sepsis is caused by a dysregulated host response to infection involving a complex (and not entirely understood) pathophysiology that leads to life-threatening organ dysfunction (Schlapbach et al. 2020). Because the mortality rate of sepsis patients continues to be comparatively high relative to non-septic patients, there is an urgent need for improved sepsis therapeutics that specifically address systemic infection and sepsis remediation, which can include modulation of the host response. Small molecule drugs that mitigate against dysregulated host responses to bacterial infection may provide the required systemic approach needed to prevent or treat sepsis.

The discovery and development of therapeutics that mitigate against host-directed mechanisms responsible for triggering sepsis, to be administered in combination with antibiotics, is a viable research and development path for improving options available for preventing morbidity and mortality associated with sepsis.

One problem with identifying new drugs or drug combinations for wound infection and sepsis is the lack of a preclinical model that has better predictive capability than cell-based systems but with higher throughput and lower cost than mammalian models (Philip et al., 2017). Therefore, a rapid, efficient screening model for sepsis therapeutics will accelerate discovery of novel therapeutics leading to improved warfighter survival and more rapid return to duty. In particular, a drug screening model that bridges the gap between poorly predictive in vitro tests and expensive, time-consuming rodent tests will greatly enhance a new development of therapeutics for sepsis by allowing determination of drug bioactivity, toxicity and off-target side effects early in the drug discovery process (Bowman and Zon, 2010), thus filling a gap between in vitro or in silico testing and pre-clinical rodent testing.

### SUMMARY OF THE INVENTION

A whole animal model such as the zebrafish allows determination of drug bioactivity, toxicity, and off-target side effects early in the drug discovery process (Bowman and Zon, 2010), thus filling a gap between in vitro or in silico testing and pre-clinical rodent testing. There are many advantages of using a zebrafish model. Zebrafish are relatively inexpensive given their small size, high fecundity, and small footprint husbandry. Zebrafish model provides an excellent opportunity for establishing a low-cost and rapid screen to accelerate discovery or selection process of potential new small molecule therapeutics for the prevention or treatment of sepsis.

In addition, pathophysiological symptoms observed in septic humans, such as dysregulated inflammatory responses (cytokine storms), tachycardia, endothelial leakage, and progressive edema have been observed in zebrafish infected with bacteria (Barber et al., 2016). Zebrafish have a high degree of genetic homology with humans (Howe et al., 2013) and have been used as a model for a range of human diseases (Lieschke and Currie, 2007). Zebrafish have demonstrated success in rapid drug screening studies (Fleming and Alderton, 2013); Patton et al. (2021) note that “Numerous drug treatments that have recently entered the clinic or clinical trials have their genesis in zebrafish.”

Zebrafish have been widely used for studying host-pathogen interactions and have an innate and adaptive immune system that becomes active at about two days and three weeks post-fertilization, respectively (Philip et al., 2017). The zebrafish immune system has many similarities to mammals (van der Sar, 2004; Sullivan and Kim, 2008; Allen and Neely, 2010; Masud et al., 2017; Torraca and Mostowy, 2018; Sisodia et al., 2020; Gomes and Mostowy, 2020), and zebrafish have been used in infection models for bacterial species of particular concern to the military, e.g., Klebsiella pneumoniae (Marcoleta et al., 2018; Leber, 2019; Zhang et al., 2019) and Pseudomonas aeruginosa (Clatworthy et al., 2009; Sullivan et al., 2017; Nogaret et al., 2021). For example, in both mammals and zebrafish, the lipopolysaccharide (LPS) component of the cell wall in Gram negative bacteria can trigger inflammatory responses (Forn-Cuní et al., 2017).

In this disclosure, to identify potential sepsis therapeutic drugs, a zebrafish endotoxicity/sepsis model was first optimized by evaluating the best combination of endpoints (mortality, tail edema, and reactive oxygen species (ROS) production), LPS sources and concentrations, and exposure times to evaluate the potential of the test drugs to provide rescue from sepsis symptoms.

After testing the zebrafish model with model compounds (fisetin, fasudil, dexamethasone, and hydrocortisone) previously shown to have efficacy in the zebrafish endotoxicity model, the optimized larval zebrafish model was used to screen 644 compounds with the rescue potential known to affect pathways implicated in the initiation and progression of sepsis in humans. These compounds were derived from commercially available libraries identified to have already been approved by the U.S. Food and Drug Administration (FDA) or other international regulatory agencies for other purposes.

Briefly, for the first stage drug screening using this zebrafish sepsis model, 3 days post-fertilization (dpf) zebrafish embryos and 5-dpf zebrafish larvae were exposed to 60 μg/mL P. aeruginosa LPS in the presence of 10 μM concentration of each compound, and after certain period of time, mortality rate and vascular leakage (tail fin edema) were determined. With the drugs showing over 80% rescue potential, the second stage screening was performed.

Through this screening were identified 16 FDA-approved drugs that showed excellent rescue from sepsis, including 5 drugs that show complete rescue of mortality and vascular damage (tail edema) in this zebrafish model.

Following LPS exposure, a neurobehavioral assay (light-dark test) was used to determine possible off-target drug side effects. Top rescue drugs were tested at a nearly 100% lethal concentration of Klebsiella pneumoniae LPS (45 μg/mL) to further evaluate rescue potential. The drug candidates with the best ability to rescue zebrafish larvae from LPS-induced endotoxicity without neurobehavioral effects are candidates for further evaluation in mammalian sepsis models.

This zebrafish model of bacterial infection might offer real time visualization of the onset and progression of infection and would also be less costly and allow for greater agility in testing combination therapies with and without antibiotics.

Therefore, proposed herein is a method to establish and validate a zebrafish model for preliminary screening of drugs and small molecules to assess their ability to prevent or treat manifestations of inflammation, sepsis, and/or septic shock caused by bacterial infection. A long-term impact of this screening effort is to facilitate the identification of approved drugs (for repurposing for a sepsis indication) or novel small molecules that can prevent initiation and onset of the physiological cascade leading to sepsis, which will contribute to a new solution for the combat medic for preventing sepsis as a complication of battlefield wound infection. In addition, use methods of drugs screened in such a way were also provided for the same purpose.

These drugs include various psychotropic drugs inhibiting the activity of serotonin receptors, histamine receptors, receptor tyrosine kinases (RTKs) of growth factor receptors, metabolism enhancing/inhibiting compounds, monoamine oxidase (MAO), and several other compounds with poorly understood mechanisms of drug activity.

One approach for the use of such compounds or compositions comprising the compounds can be a small and light auto-injector, syringe with or without a needle, ampule, or solution, lotion, gel, or cream packaged in a small container, comprising premixed formulation for parenteral or topical administration instantly after infection for the prevention of the initiation and onset of sepsis cascade or for the treatment of sepsis, and the compounds or composition can be co-administered or in parallel use with antibiotics or other anti-inflammatory drugs, including anti-viral drugs.

In view of the foregoing, methods are provided of preventing or treating severe inflammation, sepsis as well as septic shock. In a specific embodiment, a method is provided comprising administering an effective amount of a drug or combination of the drug that suppresses the activity of 5-hydroxytryptamine (5-HT, serotonin) receptors, to a subject in need of such treatment, wherein the drug can be an antagonist, a partial agonist, or an inverse agonist of 5-HT receptors, including 5-HT_{2A}, 5-HT_{2B}, or 5-HT_{2C}, and optionally 5-HT_{2A}.

In another embodiment, provided is a method involving administering an effective amount of a drug or combination of the drug that inhibits the activity of receptor tyrosine kinases (RTKs), to a subject in need of such treatment.

In another embodiment, provided is a method involving administering an effective amount of a dietary supplement or combination of the dietary supplement to a subject in need of such treatment, wherein the dietary supplement is folic acid supplement and/or iron supplement. In a specific example, the folic acid supplement is levomefolic acid and the iron supplement is ferric citrate.

According to a further embodiment, provided is a method of preventing or treating severe inflammation, sepsis or septic shock; comprising co-administering an effective amount of a drug or combination of the drug that suppresses the activity of 5-HT receptors to a subject in need of such treatment, with folic acid supplement, iron supplement, and/or RTK inhibitors.

In yet a further embodiment, provided is a method of preventing or treating severe inflammation, sepsis or septic shock; comprising administering an effective amount of a drug or combination of the drug that suppresses the activity of histamine receptors to a subject in need of such treatment, wherein the drug can be an antagonist, a partial agonist, or an inverse agonist of histamine receptors, wherein the histamine receptor can be H₁, H₂, H₃, or H₄.

A further embodiment is directed to a method of preventing or treating severe inflammation, sepsis or septic shock; comprising co-administering an effective amount of a drug or combination of the drug that suppresses the activity of histamine receptors to a subject in need of such treatment, with folic acid supplement, iron supplement, and/or RTK inhibitors.

In addition, a method of preventing or treating severe inflammation, sepsis or septic shock, comprising co-administering an effective amount a drug or combination of the drug described in this disclosure with an effective amount of at least one antibiotic can be contemplated, and optionally the drug is one or more selected from ketanserin, tegaserod, brexpiprazole, paliperidone palmitate, and sunitinib.

Further, since severe inflammation may occur due to virus infection, in particular due to a mutation-prone RNA virus infection, a method of preventing or treating severe inflammation, comprising co-administering an effective amount of a drug or combination of the drug disclosed here with an effective amount of at least one anti-viral drug, can be contemplated.

Also, since severe edema can be caused by cardiogenic shock, neurogenic shock, or anaphylactic shock, a method of treating severe edema, comprising a step of administering an effective amount a drug or combination of the drug disclosed here is contemplated.

These and other embodiments are further described in the specification below and claims.

Overall, this zebrafish model with an ability to rapidly screen multiple drugs individually, or in combination with other agents, provides an agility that cannot be achieved in higher level animal models, but that still provides the in vivo systemic interactions that cannot be achieved using in vitro models, enabling rapid screening of a wide range of drugs and drug combinations.

### BRIEF DESCRIPTION OF THE DRAWINGS

FIG. 1. Dose response curves for 3-dpf zebrafish (left graph) and 5-dpf zebrafish (right graph) to 24 hour exposure to LPS derived from E. coli 0111: B4, P. aeruginosa, and K. pneumoniae.

FIG. 2. Caudal fin morphology of 3-dpf zebrafish. A. Images depict normal caudal fin of control fish without LPS exposure. B. Images depict the caudal fin after 24 hour exposure to 60 μg/mL of P. aeruginosa LPS, resulting in a tail edema and vascular leakage.

FIG. 3. LPS dose response curves for replicate tests during the optimization phase of assay development. The left graph depicts the percentage of fish with tail edemas to increasing P. aeruginosa LPS concentrations. The right graph depicts the number of dead fish to increasing P. aeruginosa LPS concentrations. The goal was to determine a concentration of LPS that yielded close to 50% mortality while eliciting 100% combined tail edema and mortality in all fish. A concentration of 60 μg/mL of P. aeruginosa LPS was chosen for follow-on chemical screening.

FIG. 4. An example, tegaserod maleate, of the rescue results from the initial drug screening from a single 96-well plate.

FIG. 5. A graph presenting drug efficacy dose range response of the top 16 rescue drugs.

FIG. 6. A graph presenting light dark neurobehavioral testing in drug-pretreatment larva group and no drug-pretreatment larva group. The selected drugs are tegaserod maleate, ketanserin, ketanserin tartrate, and paliperidone palmitate. *Endpoint significantly different from the negative control using a 2 tailed t-test (P≤0.01). ***Endpoint had greater than 80% mortality.

FIG. 7. A graph presenting light dark neurobehavioral testing in drug-pretreatment larva group and no drug-pretreatment larva group. The selected drugs are levomefolic acid, fenspiride, pitolisant, and brexpiprazole. *Endpoint significantly different from the negative control using a 2 tailed t-test (P≤0.01). ***Endpoint had greater than 80% mortality.

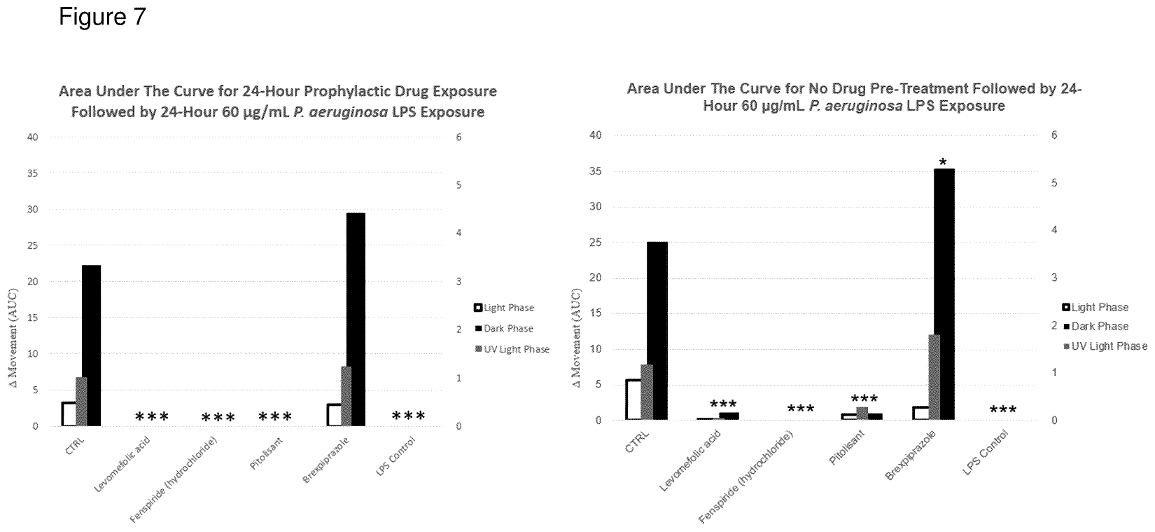

FIG. 8. Ketanserin (88) 3-dpf zebrafish initial screen under the condition of 3-dpf zebrafish P. aeruginosa LPS60 μg/mL exposure in 96 well plates.

FIG. 9. Ketanserin tartrate (437) 3-dpf zebrafish initial screen under the condition of 3-dpf zebrafish P. aeruginosa LPS60 μg/mL exposure in 96 well plates.

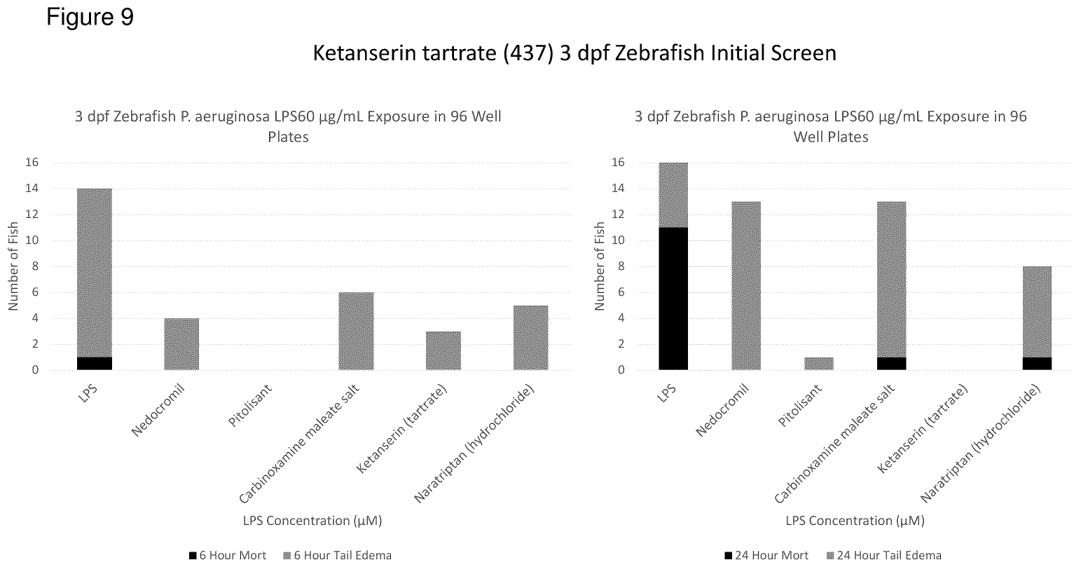

FIG. 10. Ketanserin (88) 5-dpf zebrafish screen under the condition of 5-dpf zebrafish P. aeruginosa LPS60 μg/mL exposure in 96 well plates.

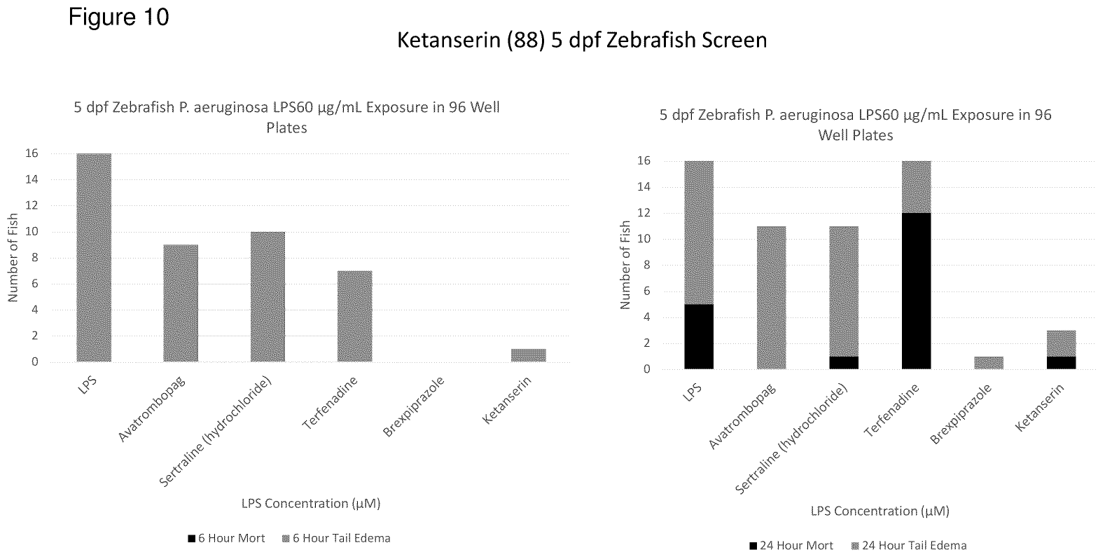

FIG. 11. Ketanserin tartrate (437) 5-dpf zebrafish screen under the condition of 5-dpf zebrafish P. aeruginosa LPS60 μg/mL exposure in 96 well plates.

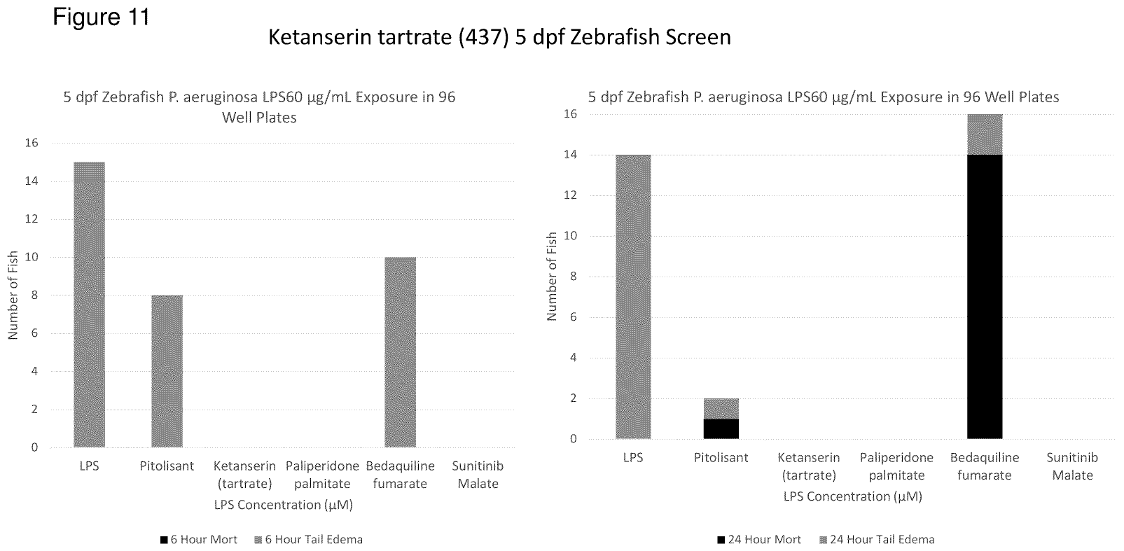

FIG. 12. Ketanserin (88) 5-dpf zebrafish dose range find under the condition of 5-dpf zebrafish P. aeruginosa LPS60 μg/mL exposure in 96 well plates.

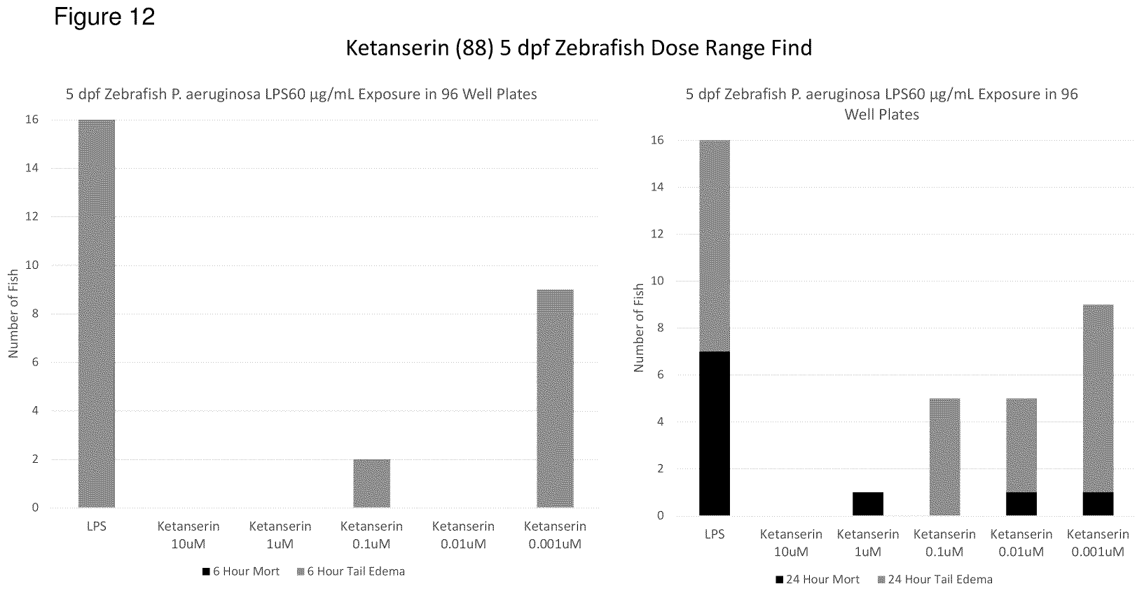

FIG. 13. Ketanserin tartrate (437) 5-dpf zebrafish dose range find under the condition of 5-dpf zebrafish P. aeruginosa LPS60 μg/mL exposure in 96 well plates.

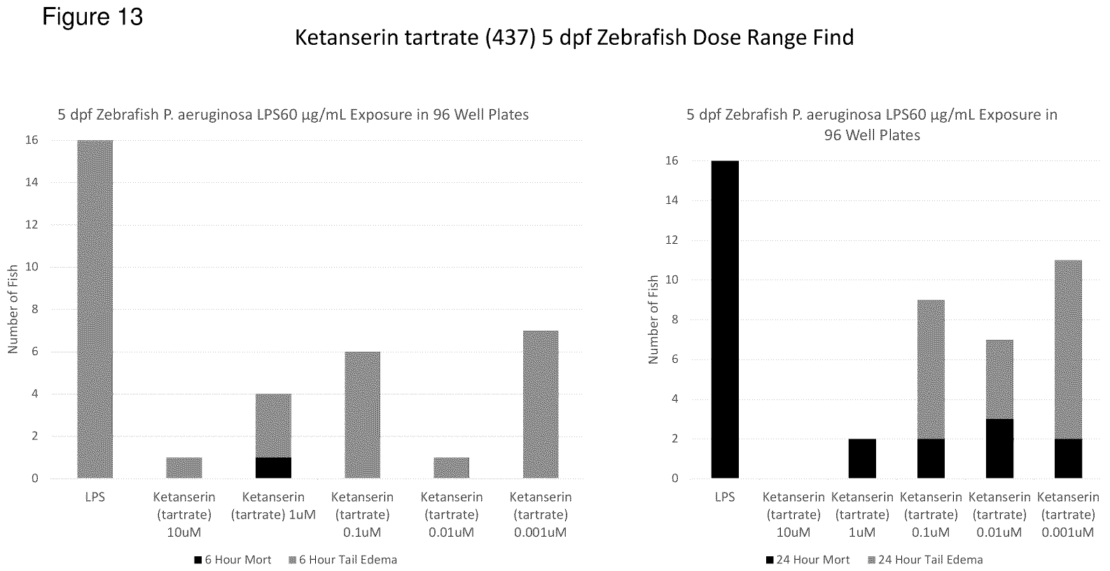

FIG. 14. Tegaserod (maleate) 3-dpf zebrafish initial screen under the condition of 3-dpf zebrafish P. aeruginosa LPS60 μg/mL exposure in 96 well plates.

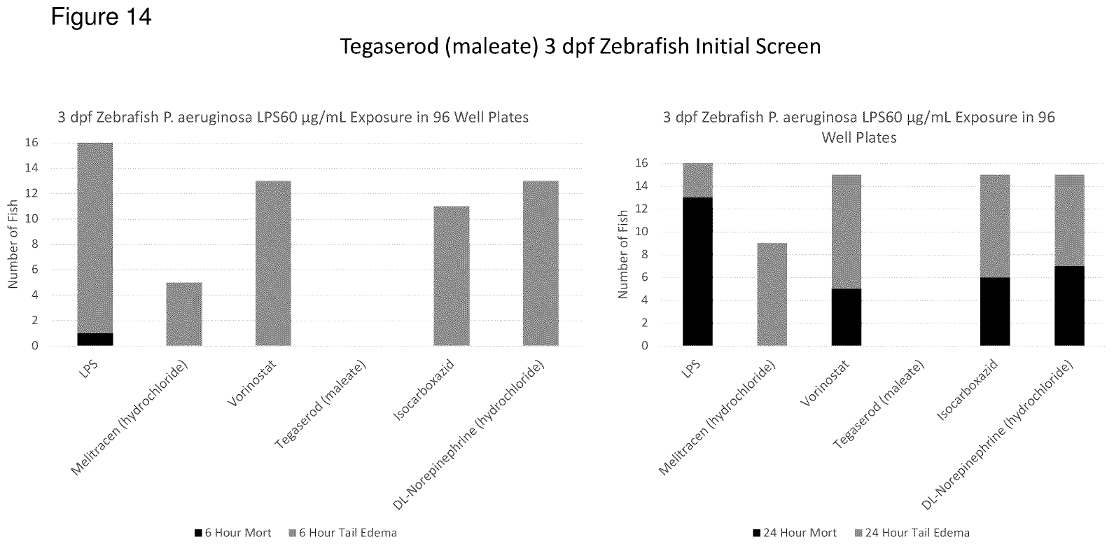

FIG. 15. Tegaserod (maleate) 5-dpf zebrafish screen under the condition of 5-dpf zebrafish P. aeruginosa LPS60 μg/mL exposure in 96 well plates.

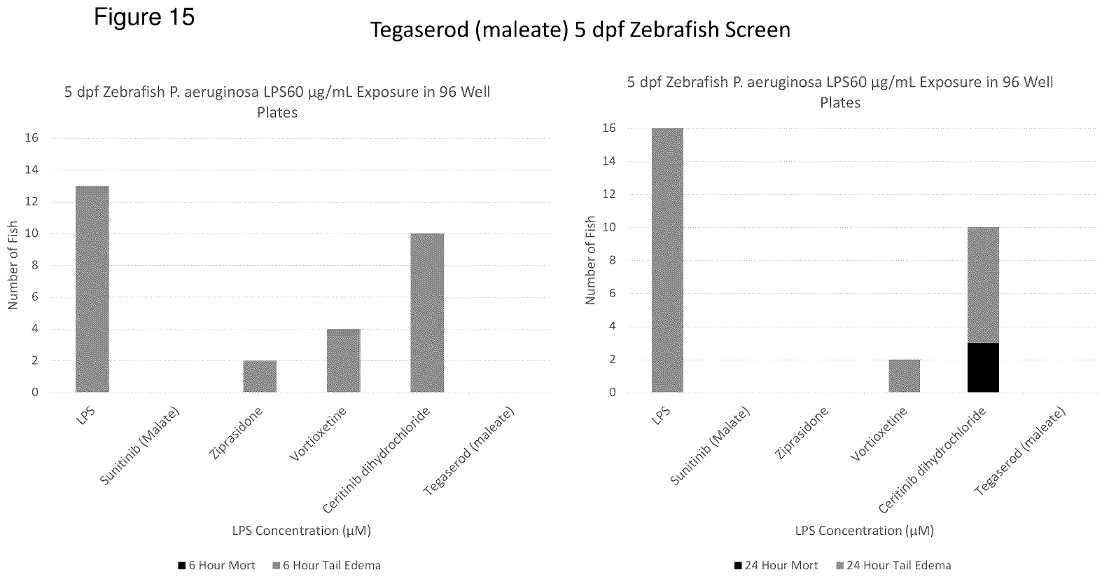

FIG. 16. Tegaserod (maleate) 5-dpf zebrafish dose range find under the condition of 5-dpf zebrafish P. aeruginosa LPS60 μg/mL exposure in 96 well plates.

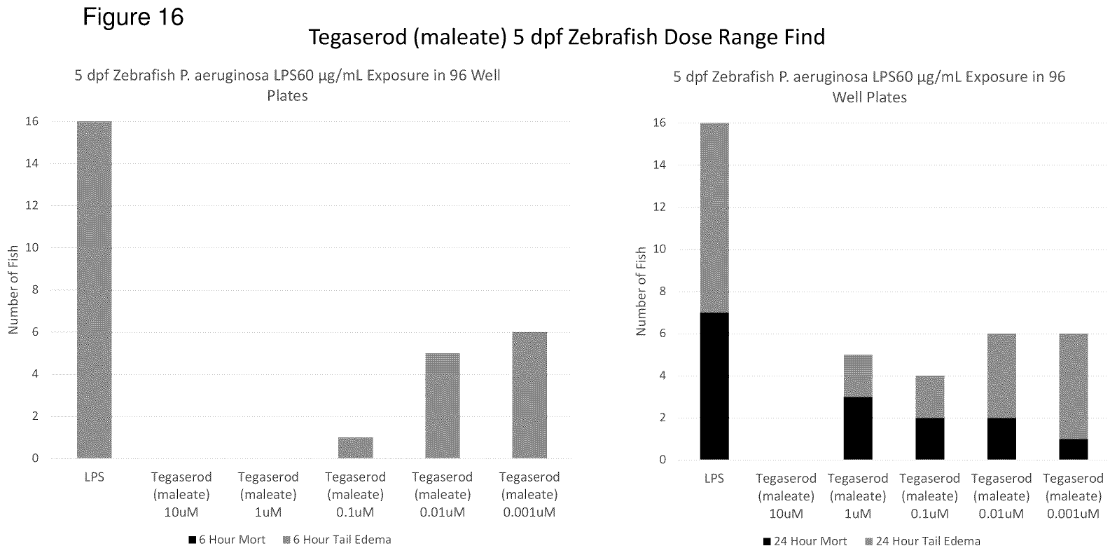

FIG. 17. Paliperidone palmitate (429) 3-dpf zebrafish initial screen under the condition of 3-dpf zebrafish P. aeruginosa LPS60 g/mL exposure in 96 well plates.

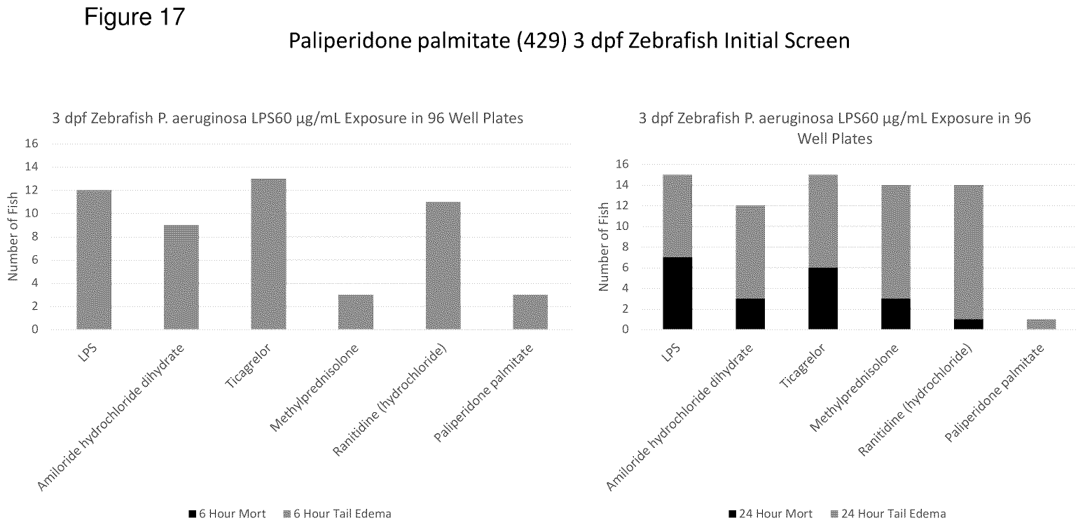

FIG. 18. Paliperidone palmitate (429) 5-dpf zebrafish screen under the condition of 5-dpf zebrafish P. aeruginosa LPS60 μg/mL exposure in 96 well plates.

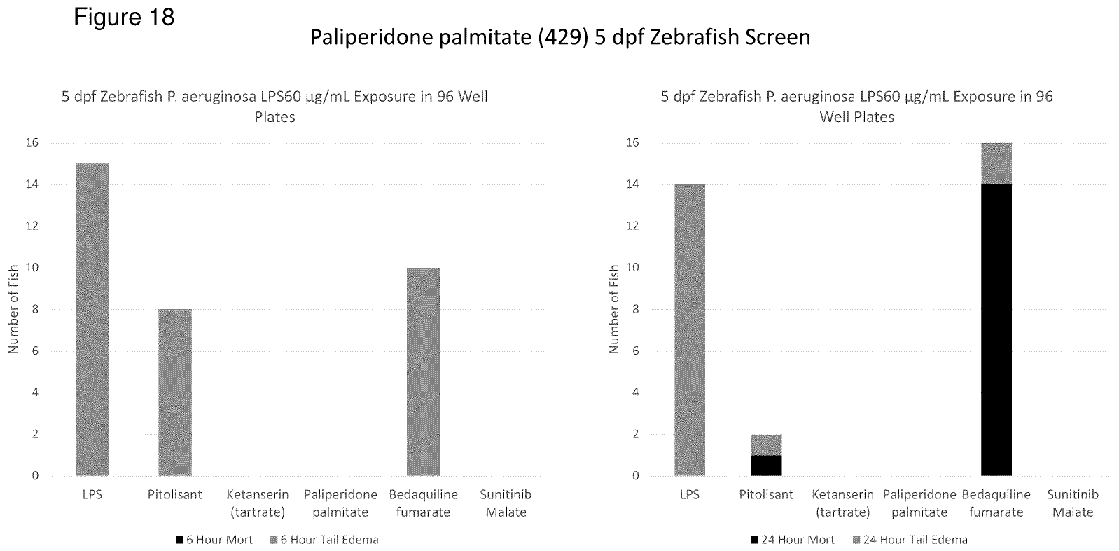

FIG. 19. Paliperidone palmitate (429) 5-dpf zebrafish dose range find under the condition of 5-dpf zebrafish P. aeruginosa LPS60 μg/mL exposure in 96 well plates.

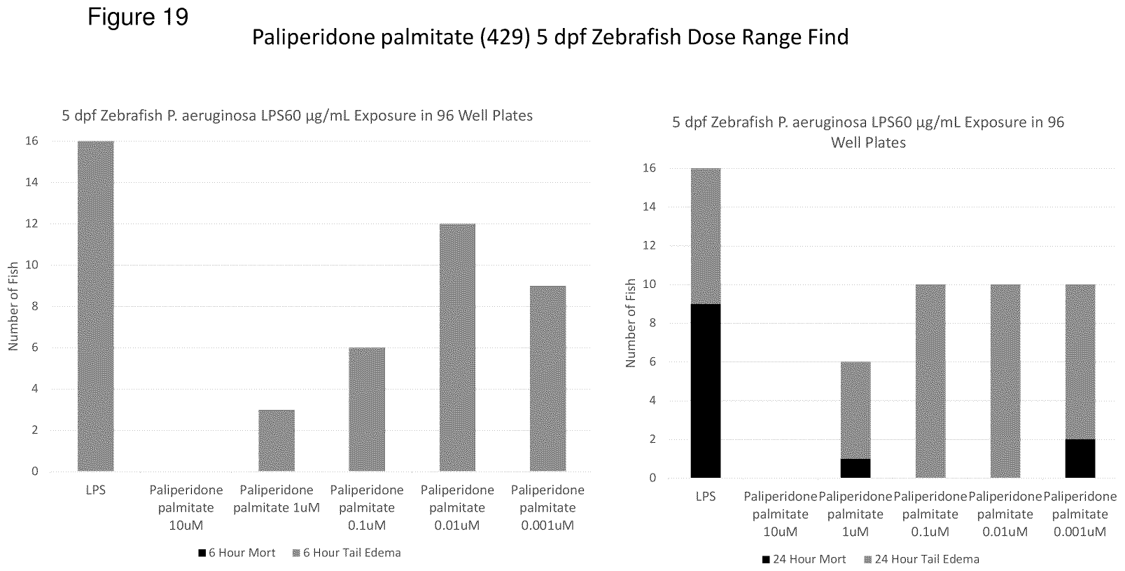

FIG. 20. Paliperidone (61) 5-dpf zebrafish dose range find under the condition of 5-dpf zebrafish P. aeruginosa LPS60 μg/mL exposure in 96 well plates. Note: Paliperidone tested for comparison to paliperidone palmitate. Paliperidone did not show good rescue at 3 dpf (6%) and was not tested during the 5 dpf screening.

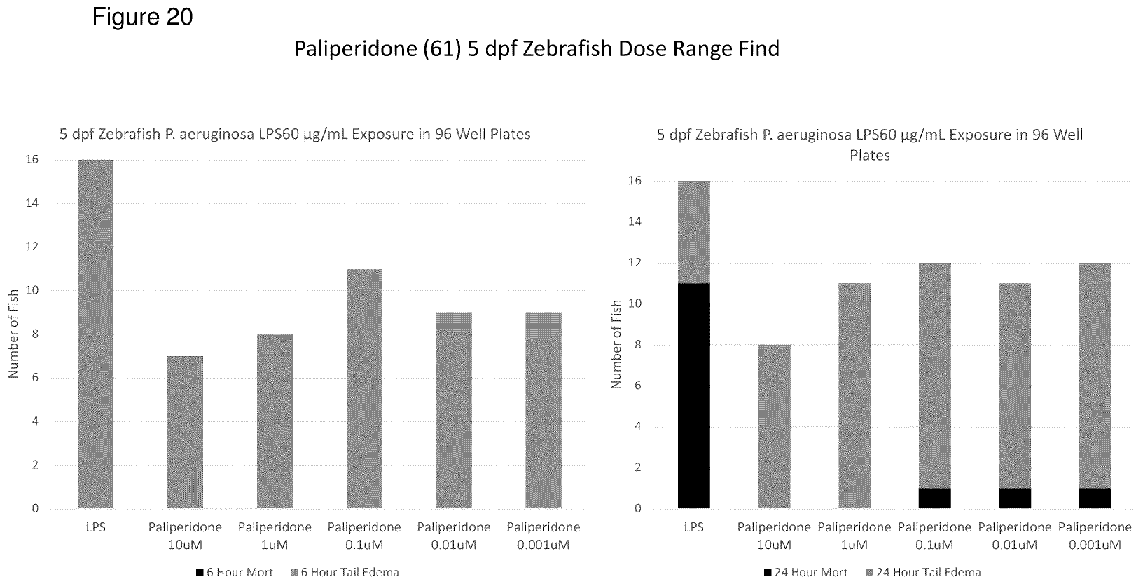

FIG. 21. Risperidone (4) 5-dpf zebrafish dose range find under the condition of 5-dpf zebrafish P. aeruginosa LPS60 μg/mL exposure in 96 well plates. Note: Palidperidone is the primary metabolite of Risperidone. Risperidone was tested for comparison to paliperidone and paliperidone palmitate. Risperidone did not show good rescue at 3 dpf (6%) and was not tested during the 5 dpf screening.

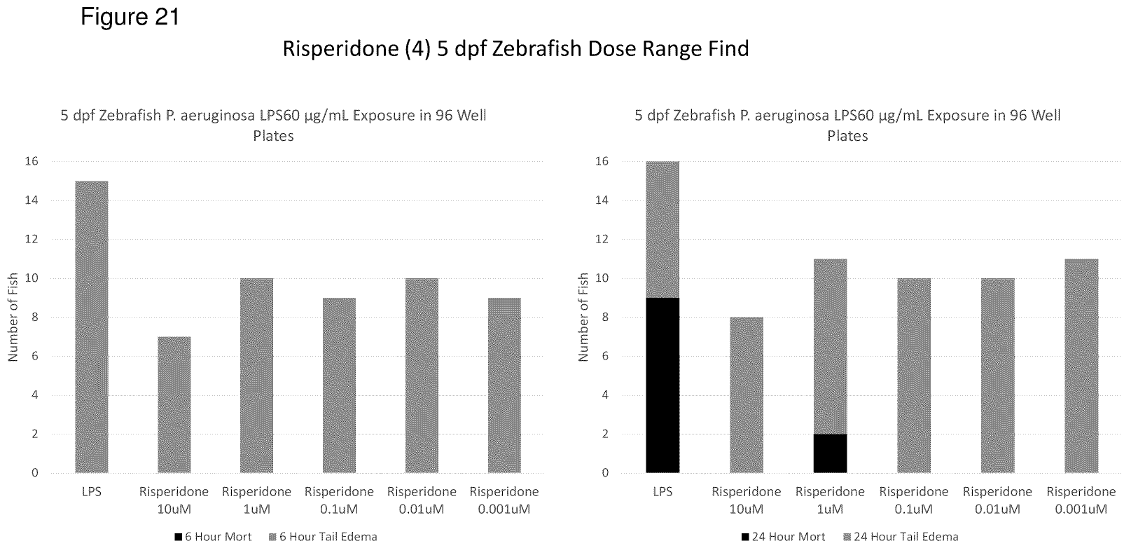

FIG. 22. Quetiapine (11) 3-dpf zebrafish initial screen under the condition of 3-dpf zebrafish P. aeruginosa LPS60 μg/mL exposure in 96 well plates.

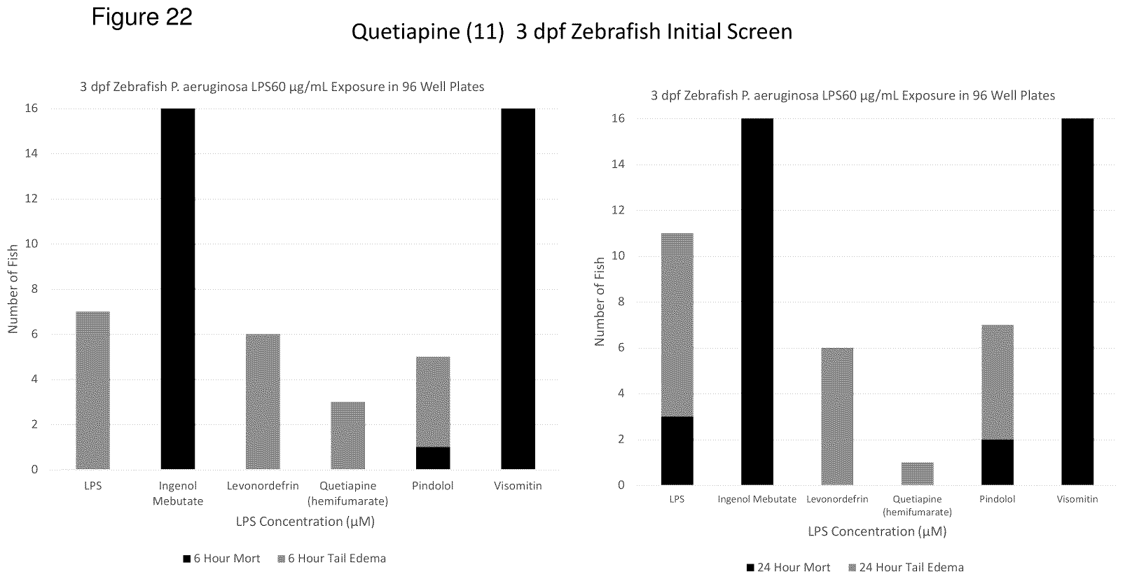

FIG. 23. Quetiapine (11) 5-dpf zebrafish screen under the condition of 5-dpf zebrafish P. aeruginosa LPS60 μg/mL exposure in 96 well plates.

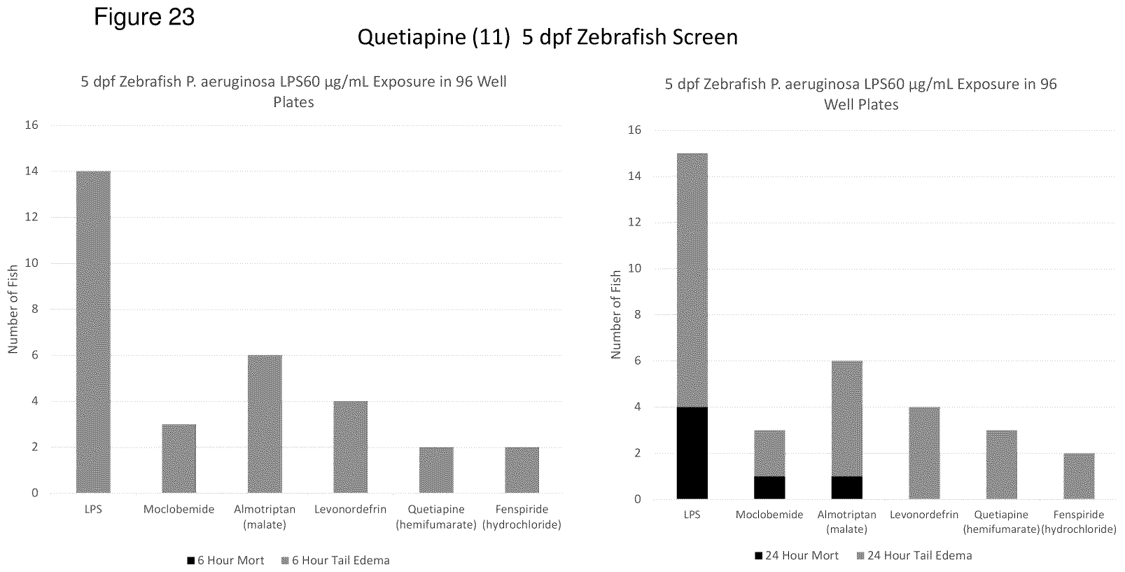

FIG. 24. Quetiapine (11) dose range find under the condition of 5-dpf zebrafish P. aeruginosa LPS60 μg/mL exposure in 96 well plates.

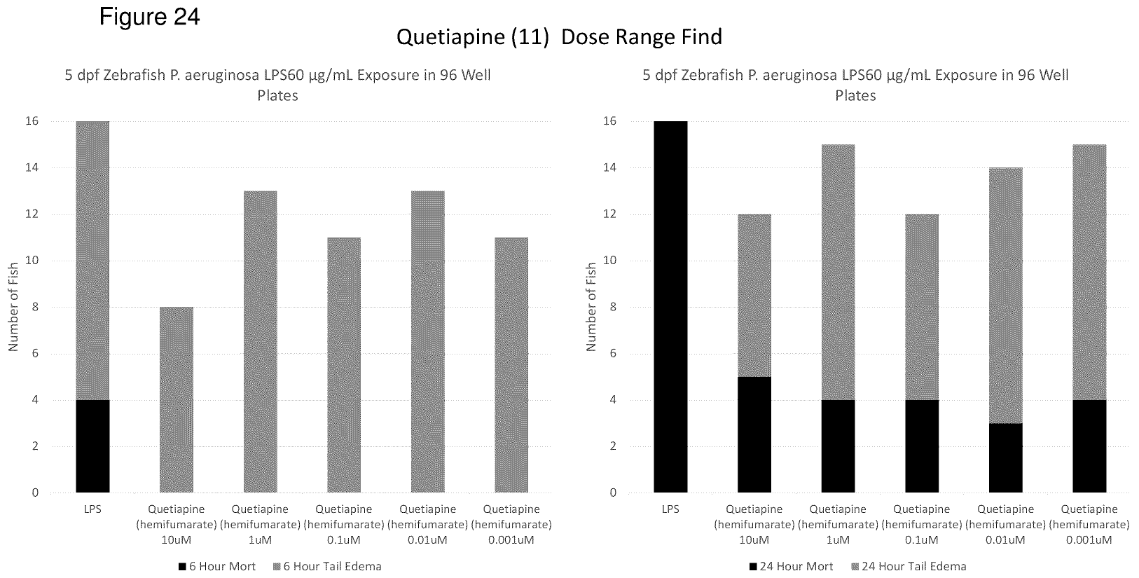

FIG. 25. Ziprasidone hydrochloride monohydrate (63) 3-dpf zebrafish initial screen under the condition of 3-dpf zebrafish P. aeruginosa LPS60 μg/mL exposure in 96 well plates.

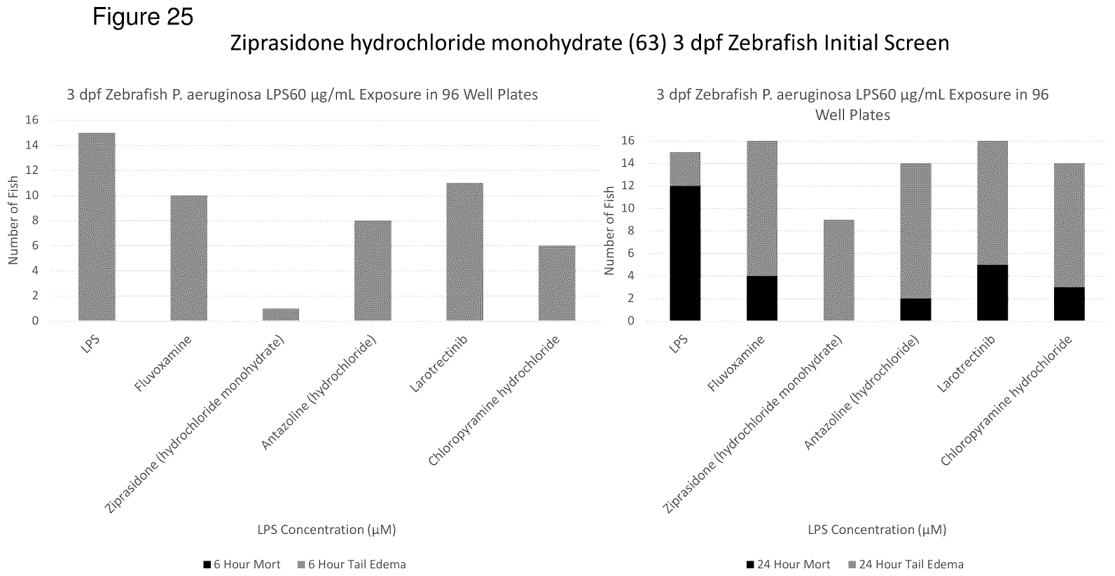

FIG. 26. Ziprasidone (361) 3-dpf zebrafish initial screen under the condition of 3-dpf zebrafish P. aeruginosa LPS60 μg/mL exposure in 96 well plates.

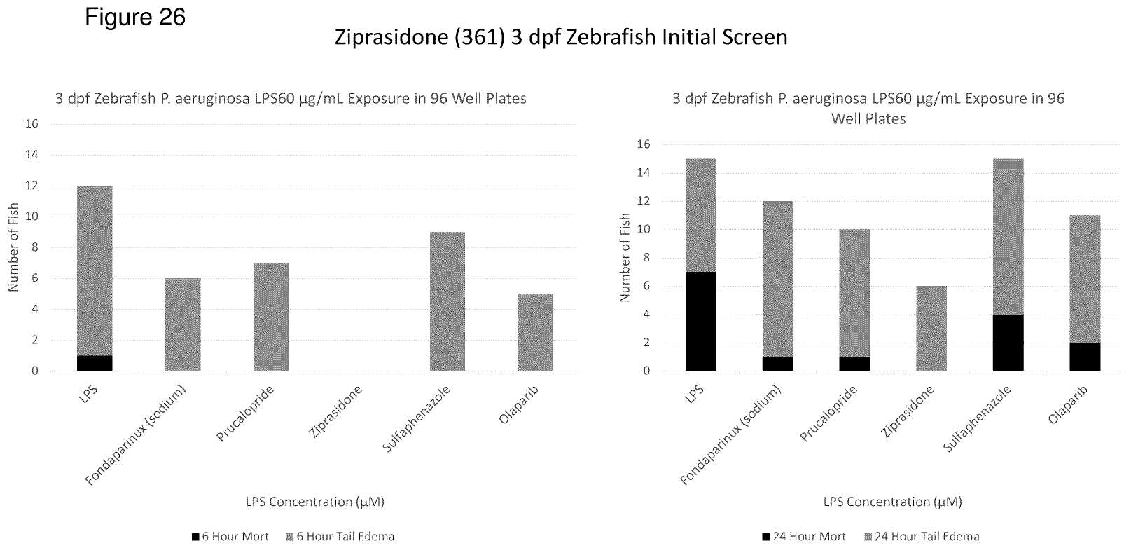

FIG. 27. Ziprasidone hydrochloride monohydrate (63) 5-dpf zebrafish screen under the condition of 5-dpf zebrafish P. aeruginosa LPS60 μg/mL exposure in 96 well plates.

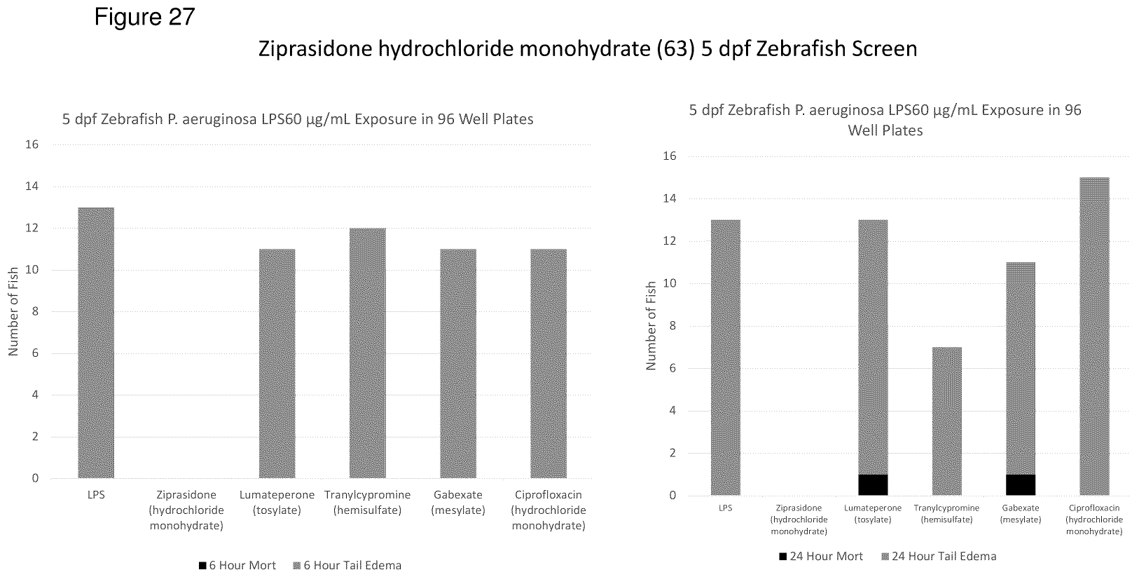

FIG. 28. Ziprasidone (361) 5-dpf zebrafish screen under the condition of 5-dpf zebrafish P. aeruginosa LPS60 μg/mL exposure in 96 well plates.

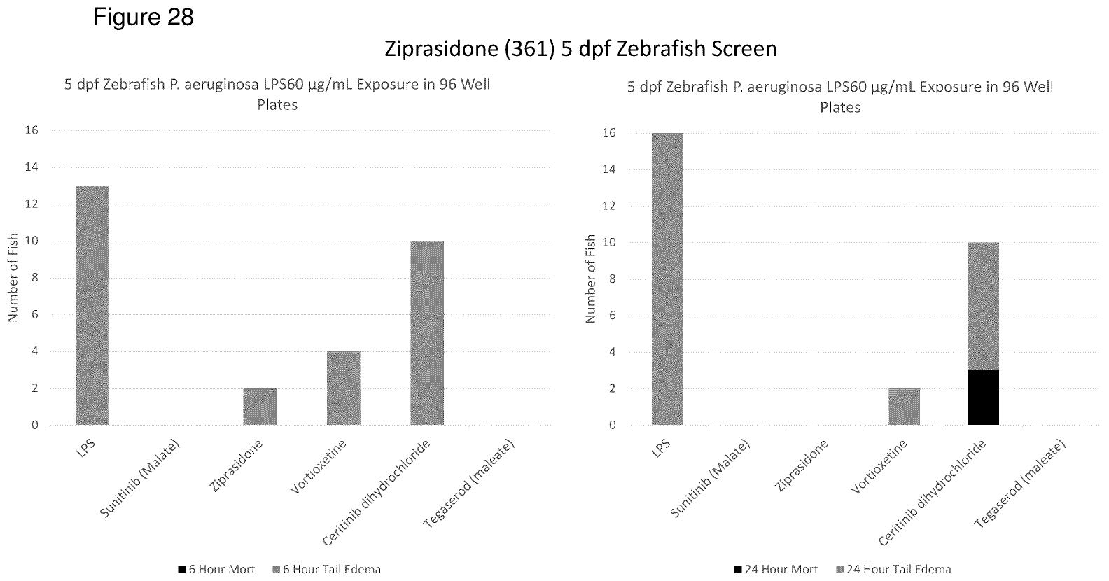

FIG. 29. Ziprasidone hydrochloride monohydrate (63) 5-dpf zebrafish dose range find under the condition of 5-dpf zebrafish P. aeruginosa LPS60 μg/mL exposure in 96 well plates.

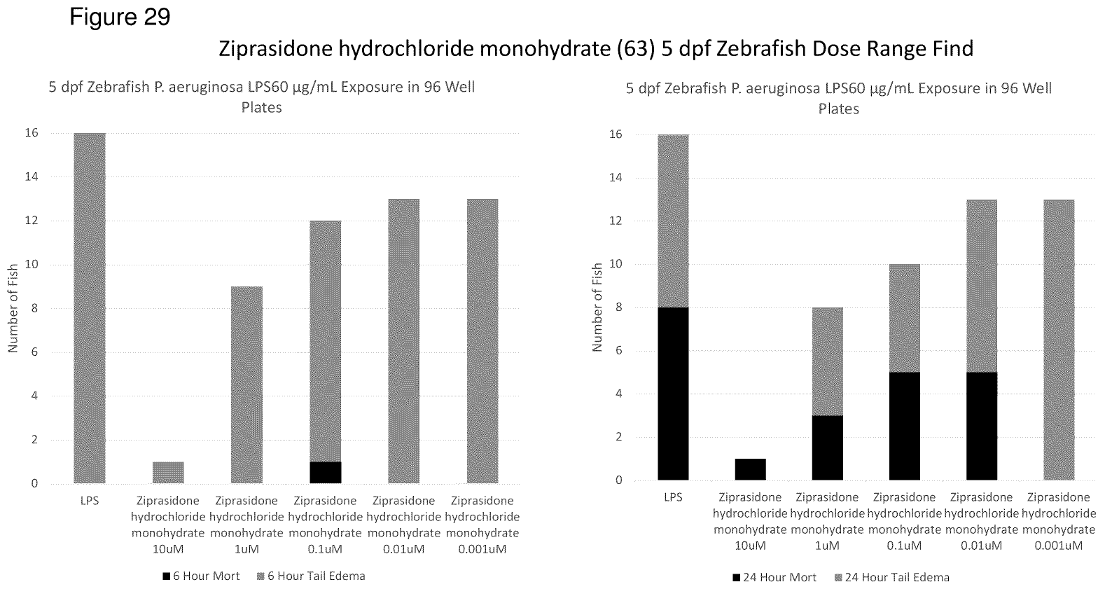

FIG. 30. Ziprasidone (361) 5-dpf zebrafish dose range find under the condition of 5-dpf zebrafish P. aeruginosa LPS60 μg/mL exposure in 96 well plates.

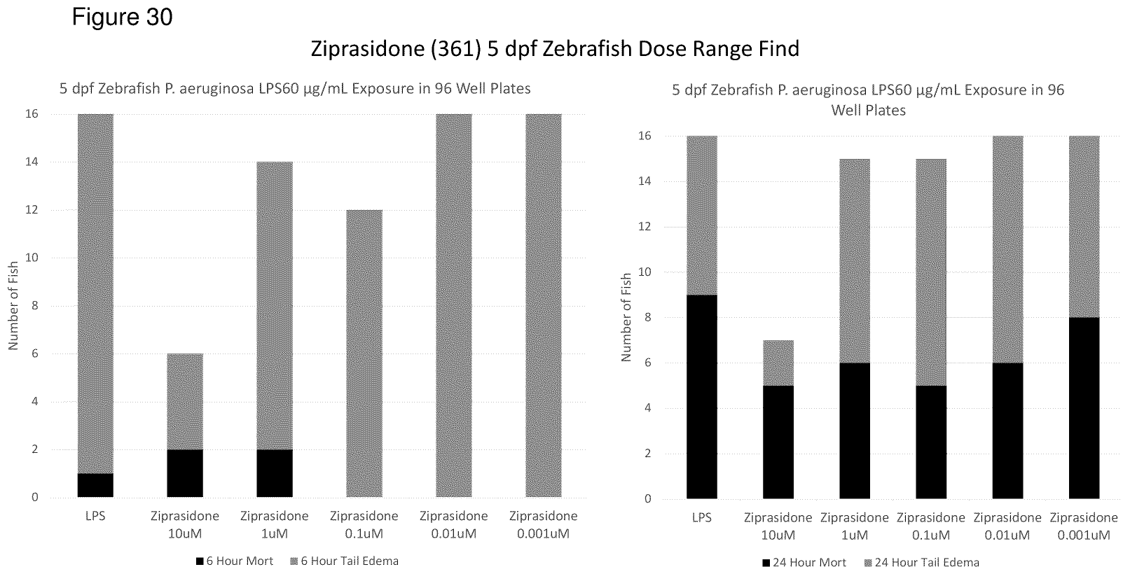

FIG. 31. Brexpiprazole (45) 3-dpf zebrafish initial screen under the condition of 3-dpf zebrafish P. aeruginosa LPS60 μg/mL exposure in 96 well plates. Note: Terfenadine fish were alive at 24 hours but had pericardial edemas. Fexofenadine (compound 137) which is the metabolic derivative of terfenadine did not rescue tail edema but did rescue mortality.

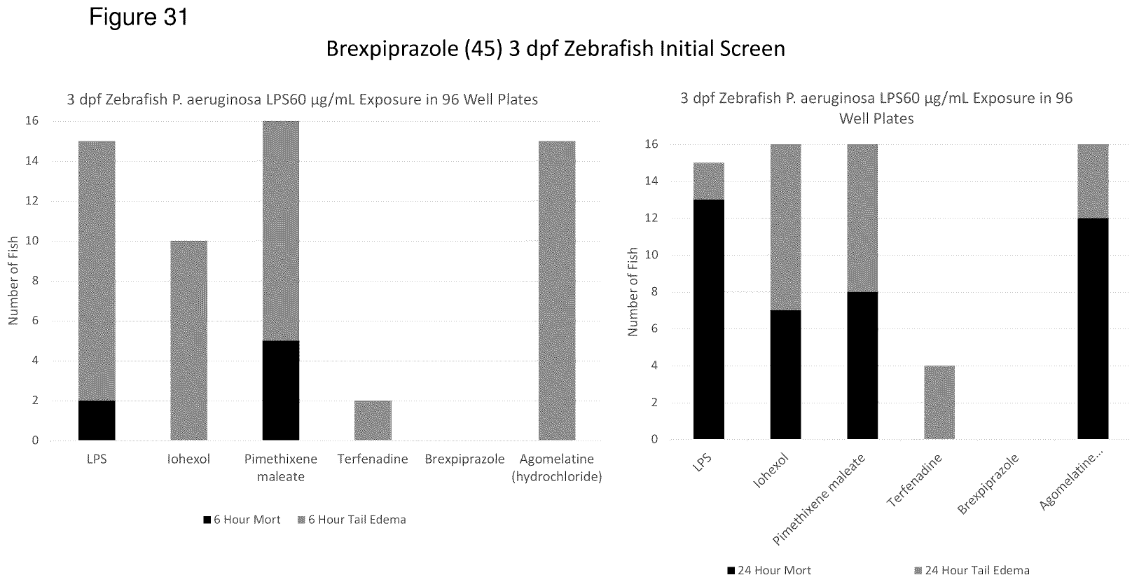

FIG. 32. Brexpiprazole (45) 5-dpf zebrafish screen under the condition of 5-dpf zebrafish P. aeruginosa LPS60 μg/mL exposure in 96 well plates.

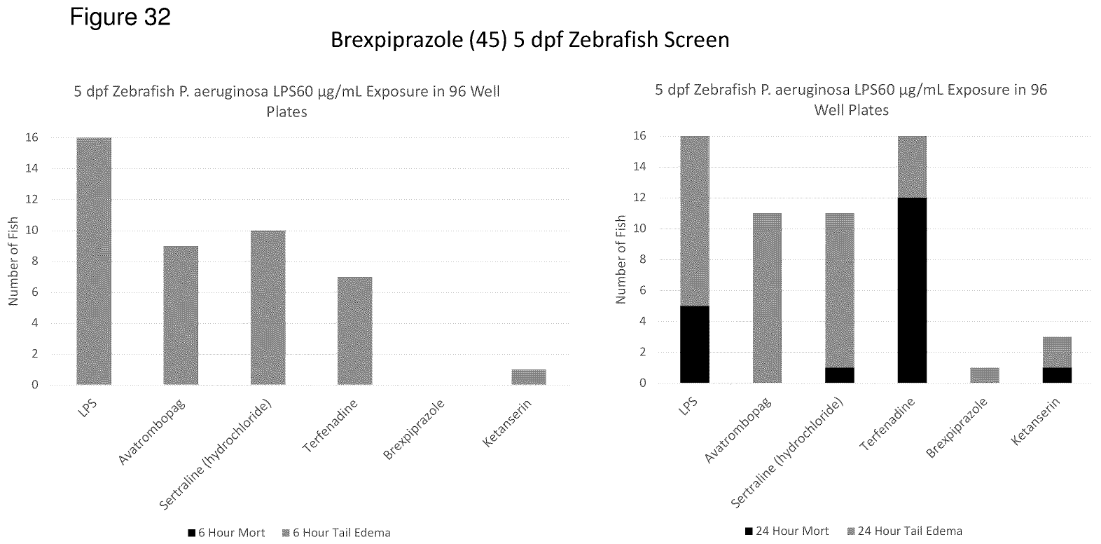

FIG. 33. Brexpiprazole (45) 5-dpf zebrafish dose range find under the condition of 5-dpf zebrafish P. aeruginosa LPS60 g/mL exposure in 96 well plates.

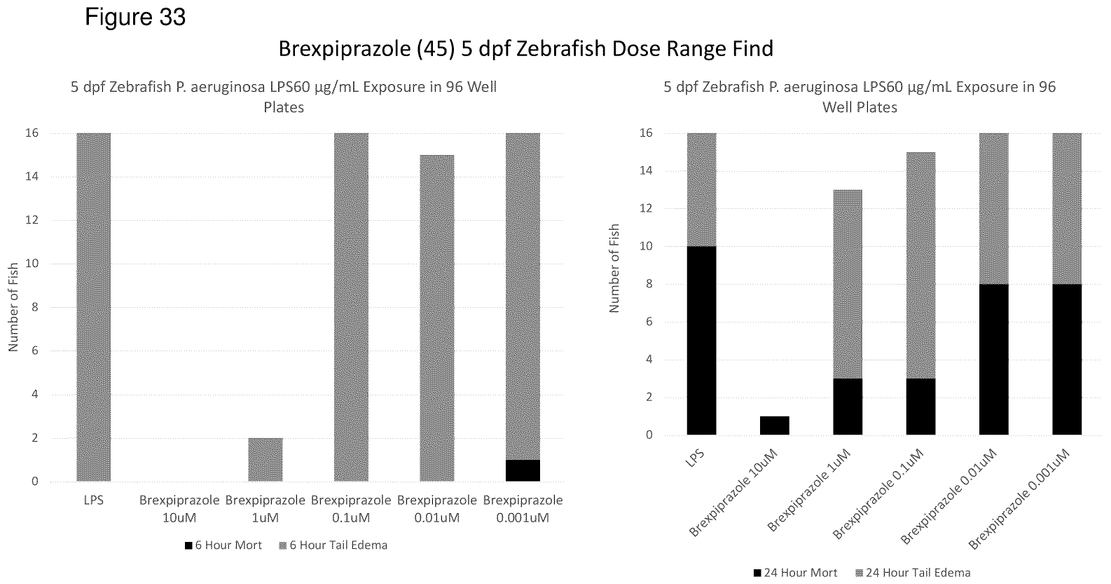

FIG. 34. Sunitinib (97) 3-dpf zebrafish initial screen under the condition of 3-dpf zebrafish P. aeruginosa LPS60 μg/mL exposure in 96 well plates.

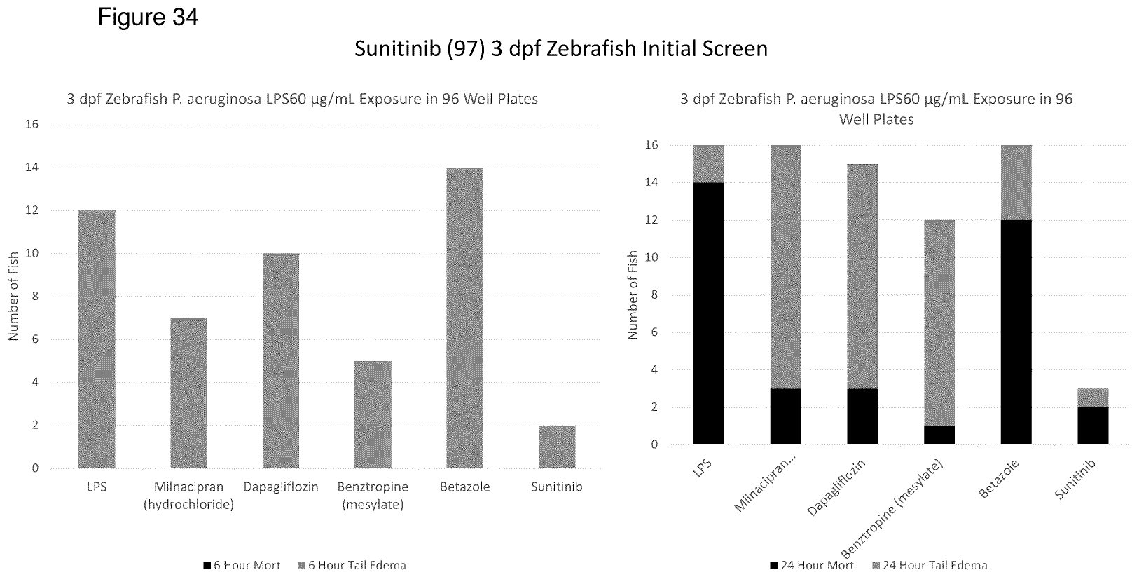

FIG. 35. Sunitinib malate (354) 3-dpf zebrafish initial screen under the condition of 3-dpf zebrafish P. aeruginosa LPS60 μg/mL exposure in 96 well plates.

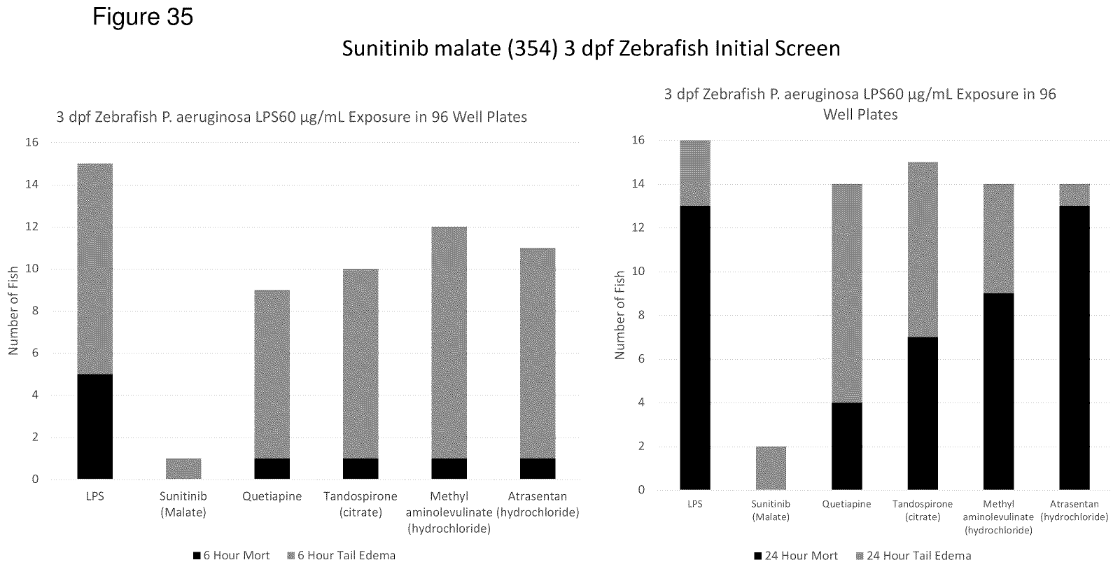

FIG. 36. Sunitinib malate (621) 3-dpf zebrafish initial screen under the condition of 3-dpf zebrafish P. aeruginosa LPS60 μg/mL exposure in 96 well plates.

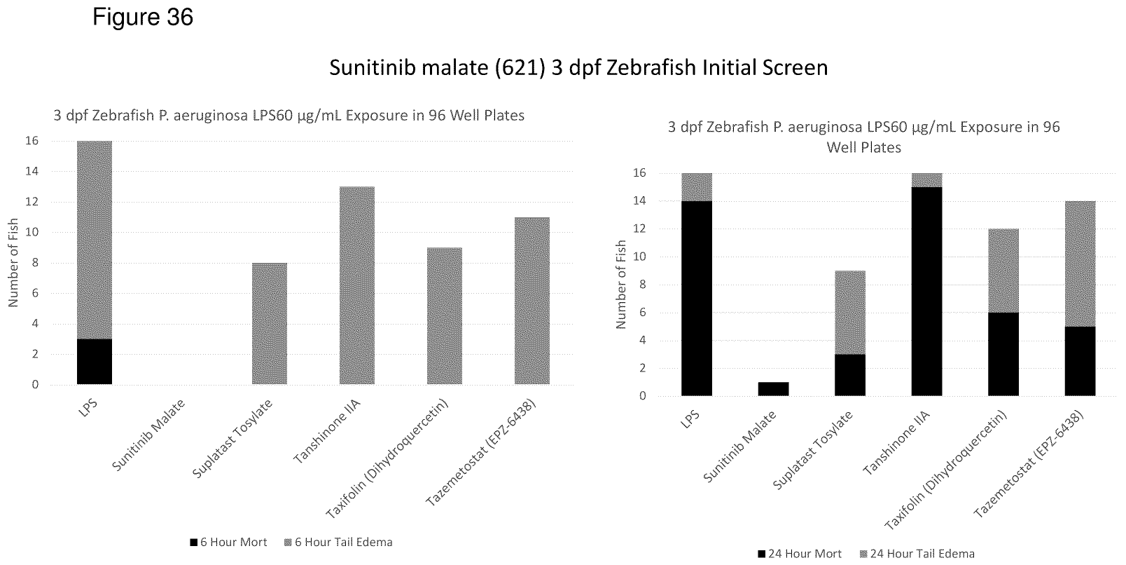

FIG. 37. Sunitinib (97) 5-dpf zebrafish screen under the condition of 5-dpf zebrafish P. aeruginosa LPS60 μg/mL exposure in 96 well plates.

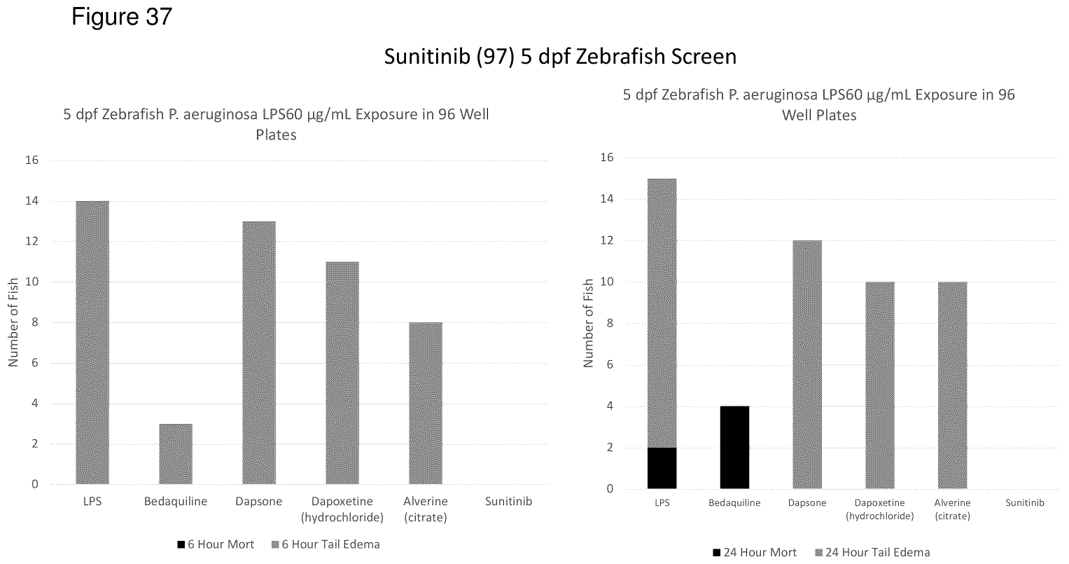

FIG. 38. Sunitinib malate (354) 5-dpf zebrafish screen under the condition of 5-dpf zebrafish P. aeruginosa LPS60 μg/mL exposure in 96 well plates.

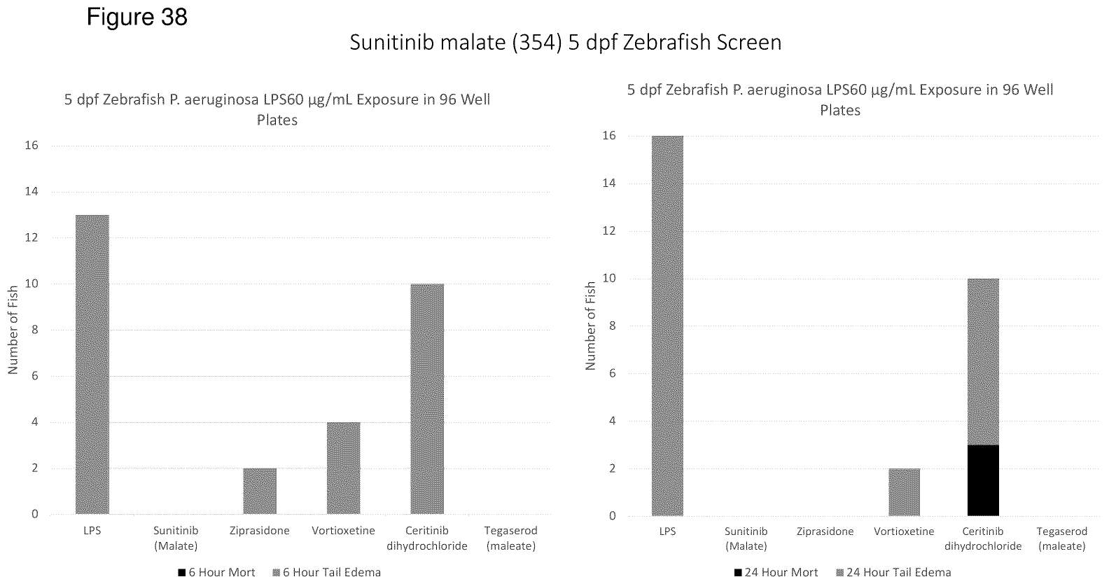

FIG. 39. Sunitinib malate (621) 5-dpf zebrafish screen under the condition of 5-dpf zebrafish P. aeruginosa LPS60 μg/mL exposure in 96 well plates.

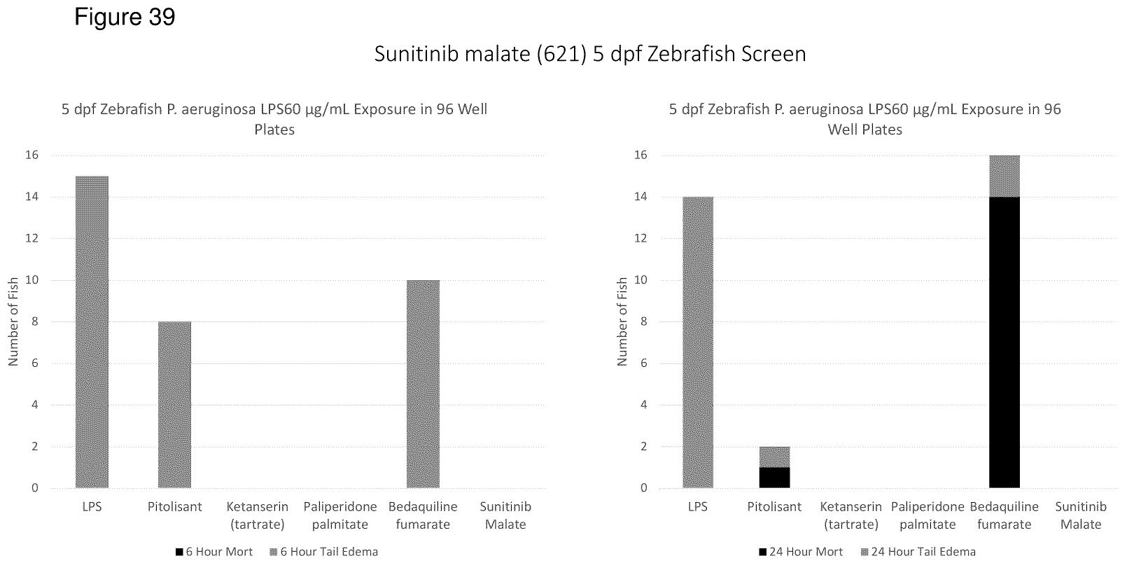

FIG. 40. Sunitinib (97) 5-dpf zebrafish dose range find under the condition of 5-dpf zebrafish P. aeruginosa LPS60 μg/mL exposure in 96 well plates.

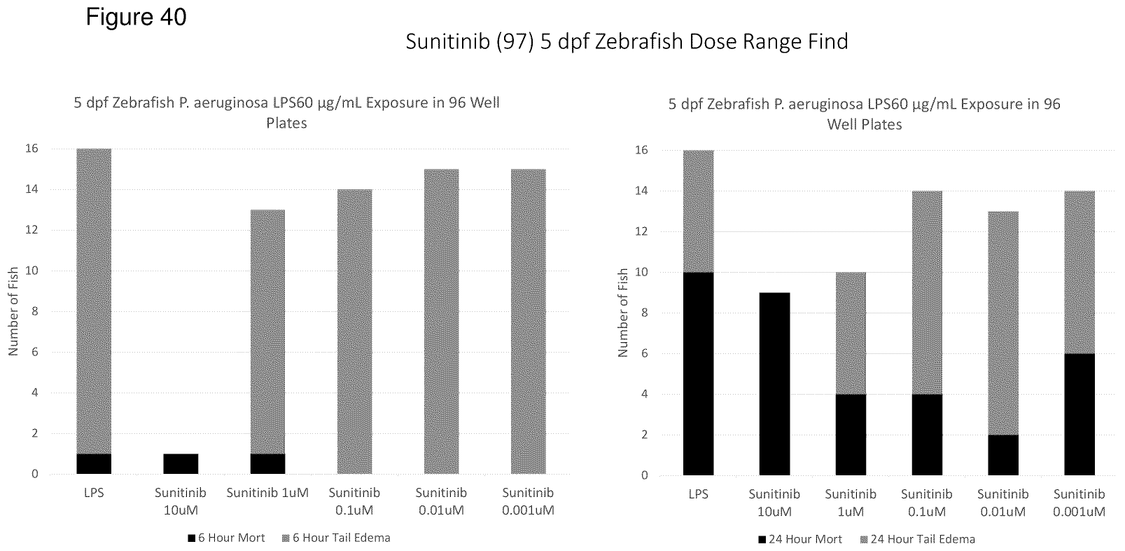

FIG. 41. Sunitinib malate (354) 5-dpf zebrafish dose range find under the condition of 5-dpf zebrafish P. aeruginosa LPS60 μg/mL exposure in 96 well plates.

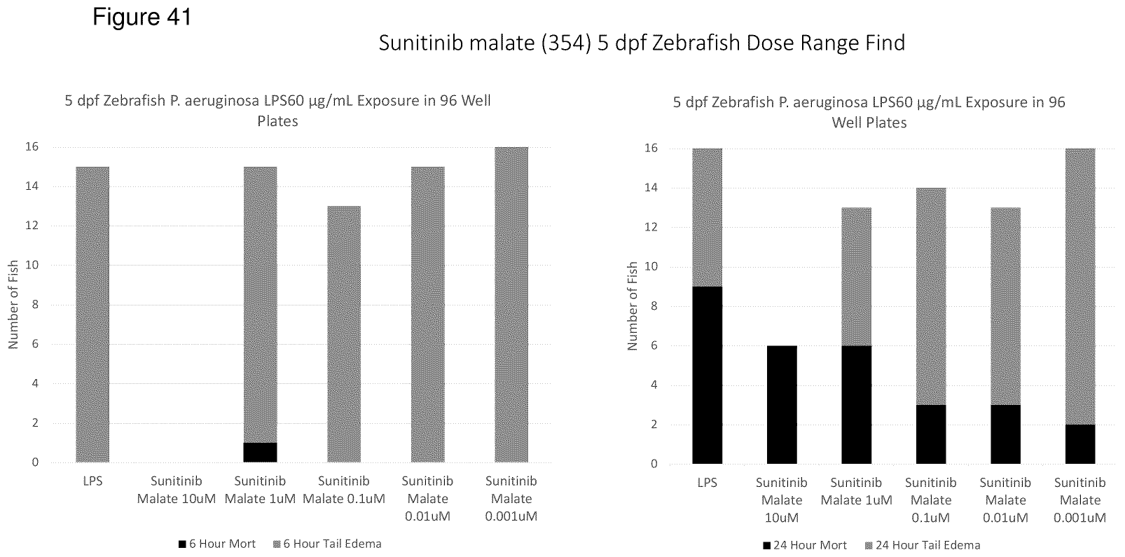

FIG. 42. Sunitinib malate (621) 5-dpf zebrafish dose range find under the condition of 5-dpf zebrafish P. aeruginosa LPS60 μg/mL exposure in 96 well plates.

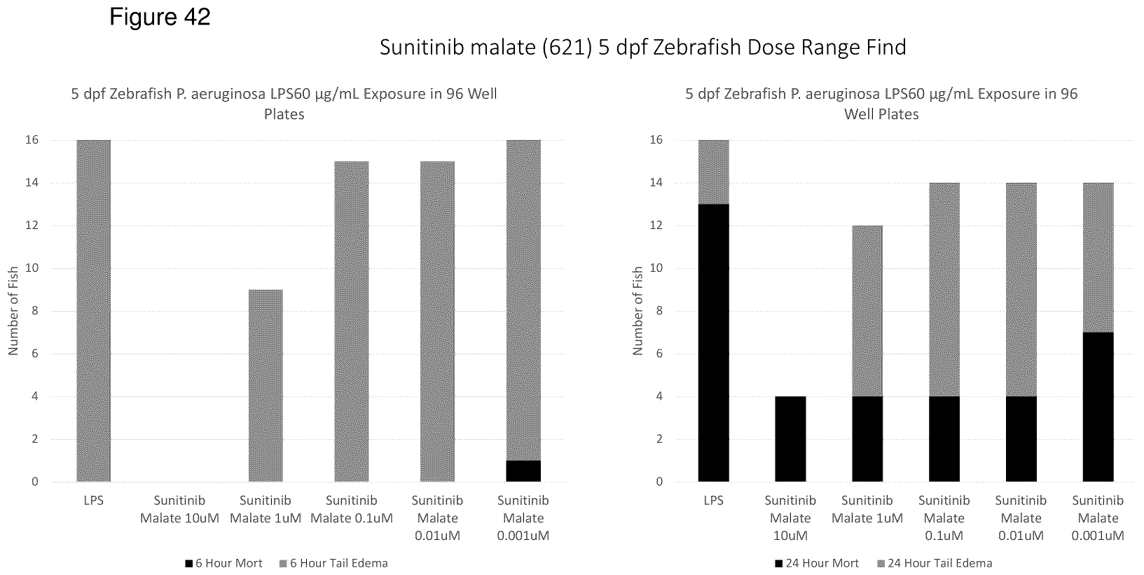

FIG. 43. Pitolisant (435) 3-dpf zebrafish initial screen under the condition of 3-dpf zebrafish P. aeruginosa LPS60 μg/mL exposure in 96 well plates.

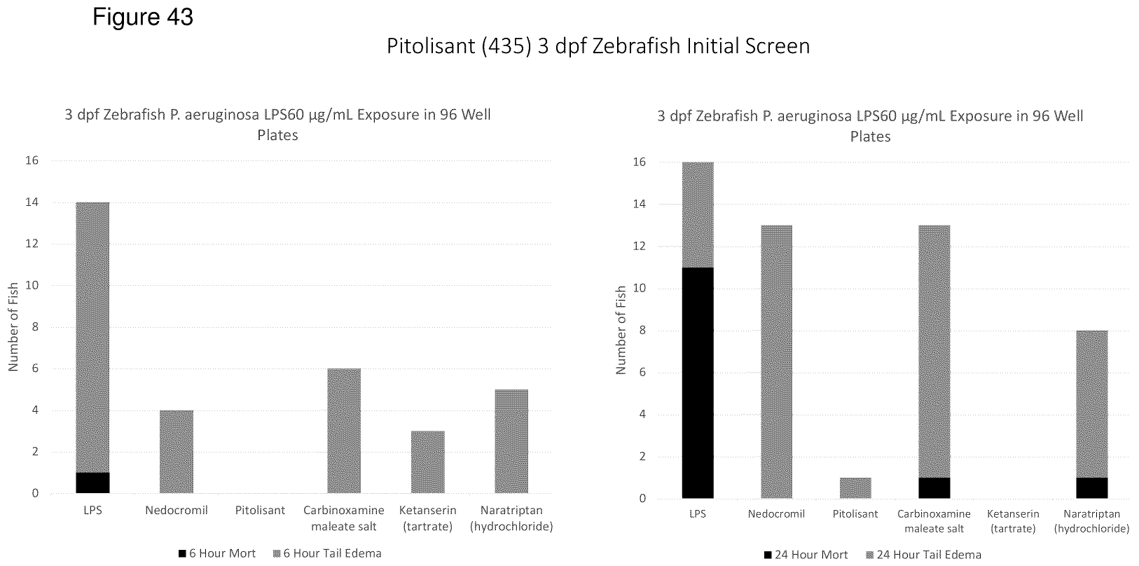

FIG. 44. Pitolisant (435) 5-dpf zebrafish screen under the condition of 5-dpf zebrafish P. aeruginosa LPS60 μg/mL exposure in 96 well plates.

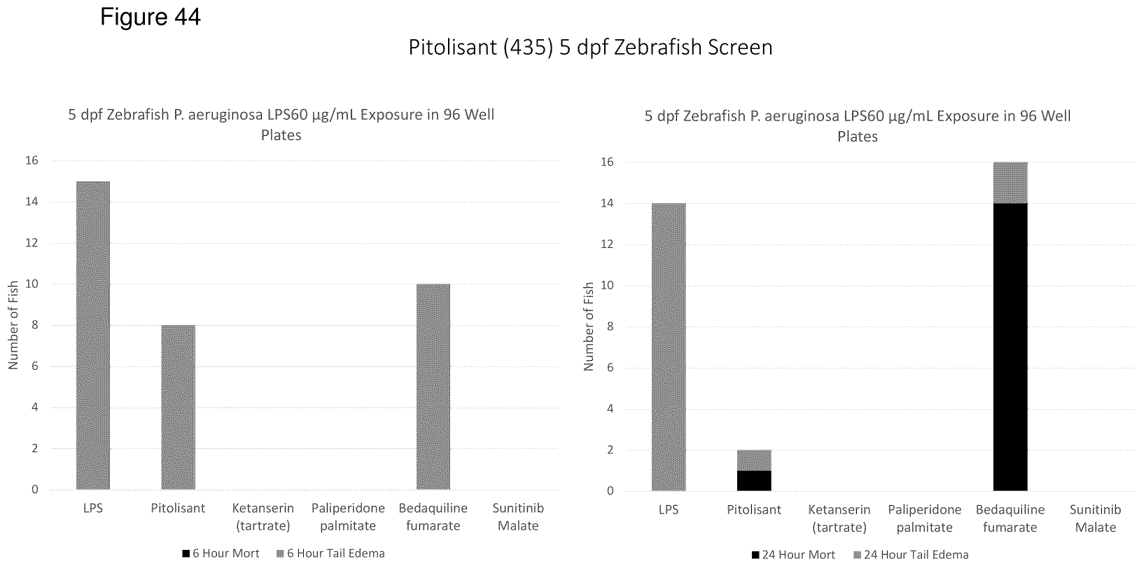

FIG. 45. Pitolisant (435) dose range find under the condition of 5-dpf zebrafish P. aeruginosa LPS60 μg/mL exposure in 96 well plates.

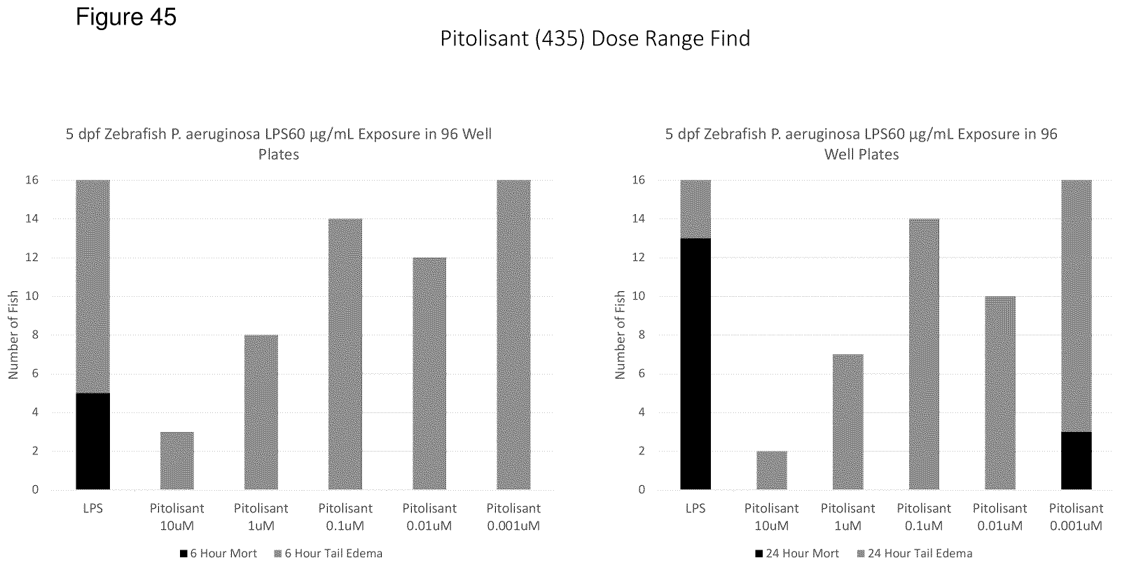

FIG. 46. Pitolisant hydrochloride (126) dose range find under the condition of 5-dpf zebrafish P. aeruginosa LPS60 μg/mL exposure in 96 well plates.

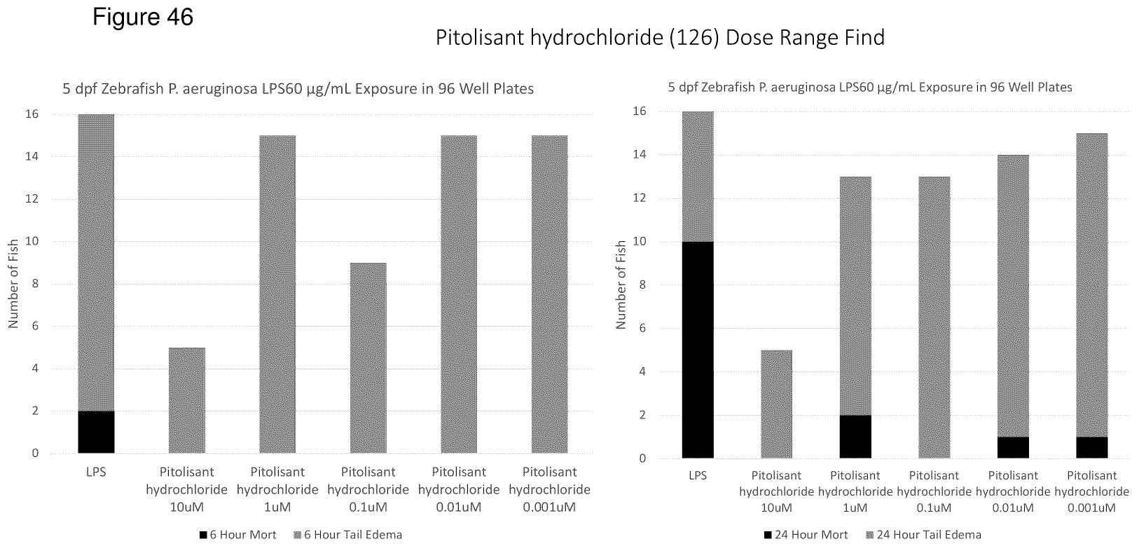

FIG. 47. Pitolisant hydrochloride (589) dose range find under the condition of 5-dpf zebrafish P. aeruginosa LPS60 μg/mL exposure in 96 well plates.

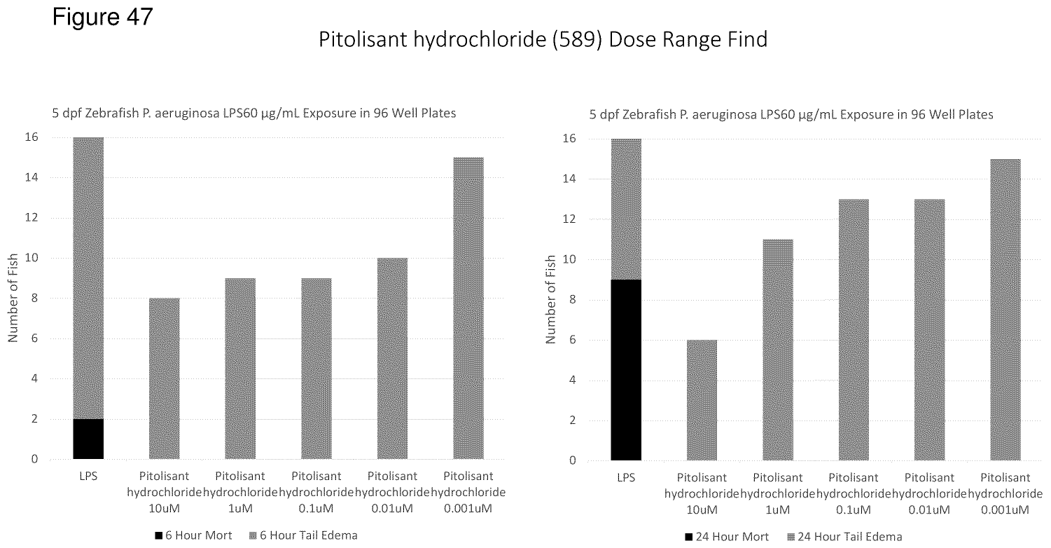

FIG. 48. Fenspiride hydrochloride (Compound 17) 3-dpf zebrafish initial screen under the condition of 3-dpf zebrafish P. aeruginosa LPS60 μg/mL exposure in 96 well plates.

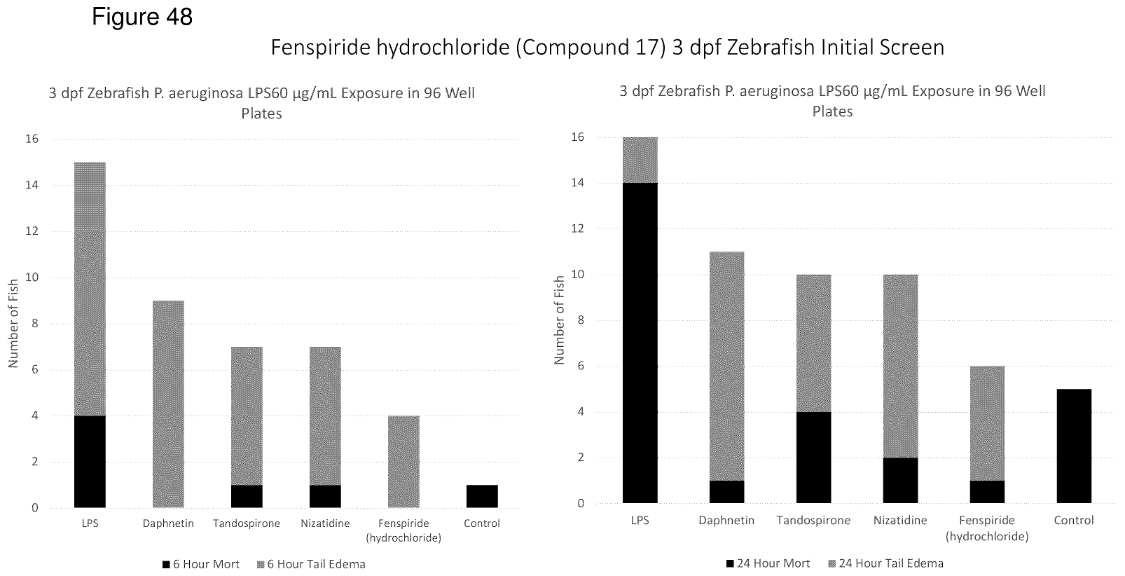

FIG. 49. Fenspiride hydrochloride (Compound 17) 5-dpf zebrafish screen under the condition of 5-dpf zebrafish P. aeruginosa LPS60 μg/mL exposure in 96 well plates.

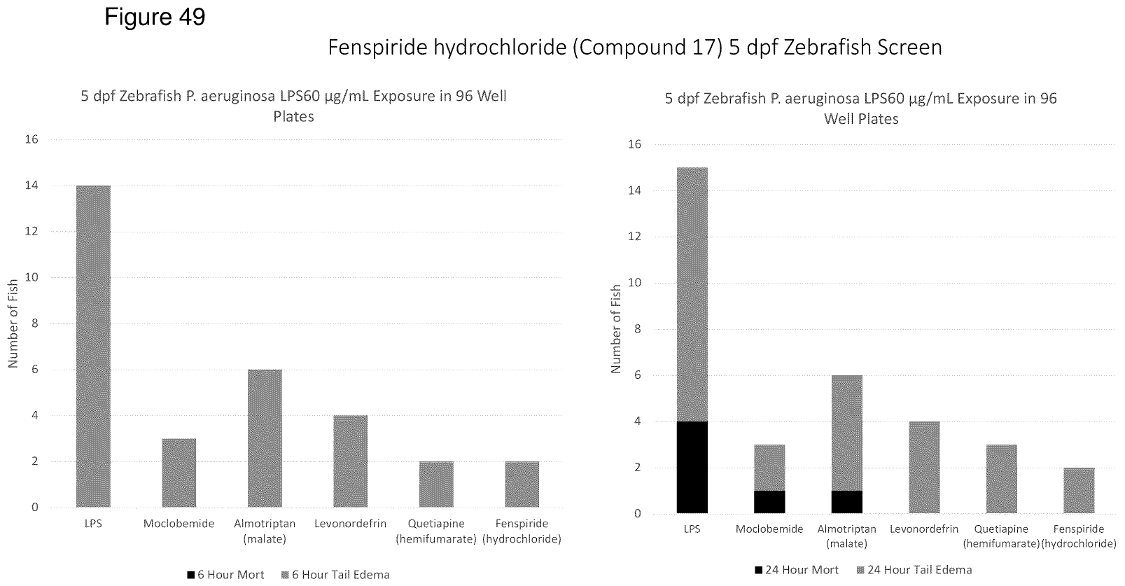

FIG. 50. Fenspiride hydrochloride (Compound 17) dose range find under the condition of 5-dpf zebrafish P. aeruginosa LPS60 μg/mL exposure in 96 well plates.

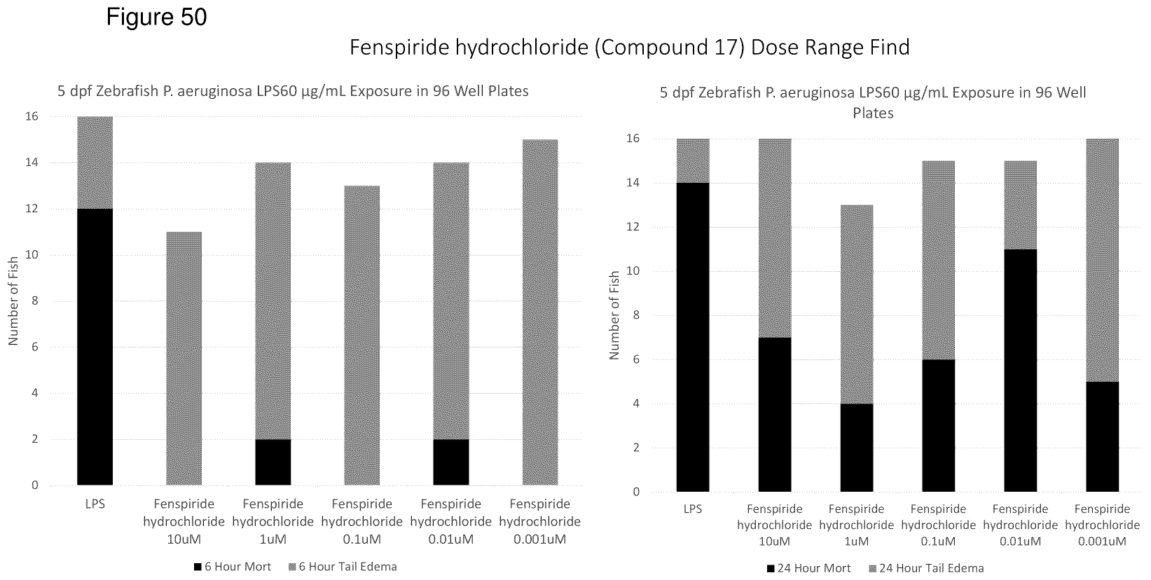

FIG. 51. Levomepromazine (316) 3-dpf zebrafish initial screen under the condition of 3-dpf zebrafish P. aeruginosa LPS60 μg/mL exposure in 96 well plates.

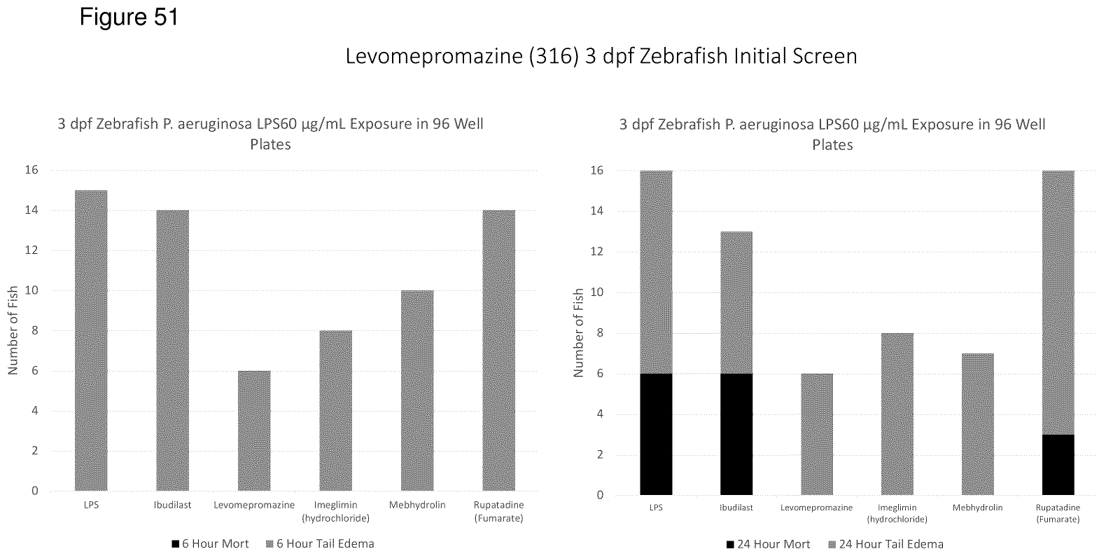

FIG. 52. Levomepromazine (316) 5-dpf zebrafish screen under the condition of 5-dpf zebrafish P. aeruginosa LPS60 μg/mL exposure in 96 well plates.

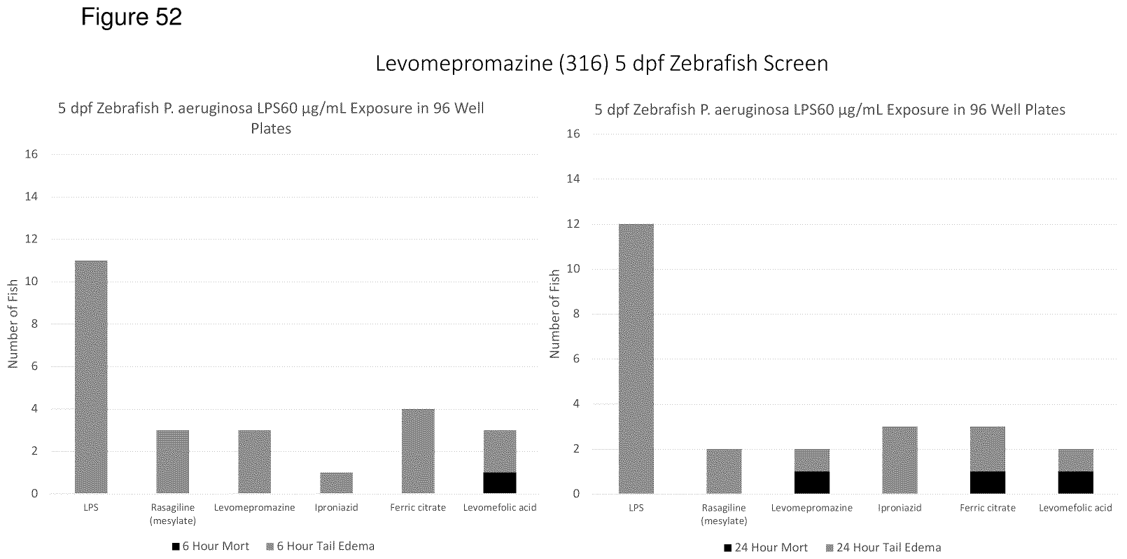

FIG. 53. Levomepromazine (316) dose range find under the condition of 5-dpf zebrafish P. aeruginosa LPS60 μg/mL exposure in 96 well plates.

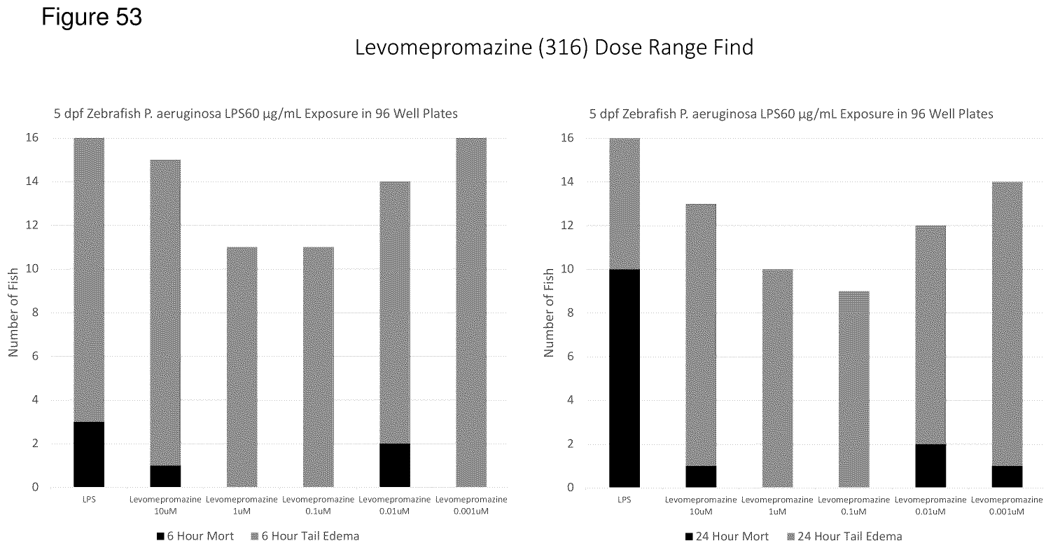

FIG. 54. Levomefolic acid (344) 3-dpf zebrafish initial screen under the condition of 3-dpf zebrafish P. aeruginosa LPS60 μg/mL exposure in 96 well plates.

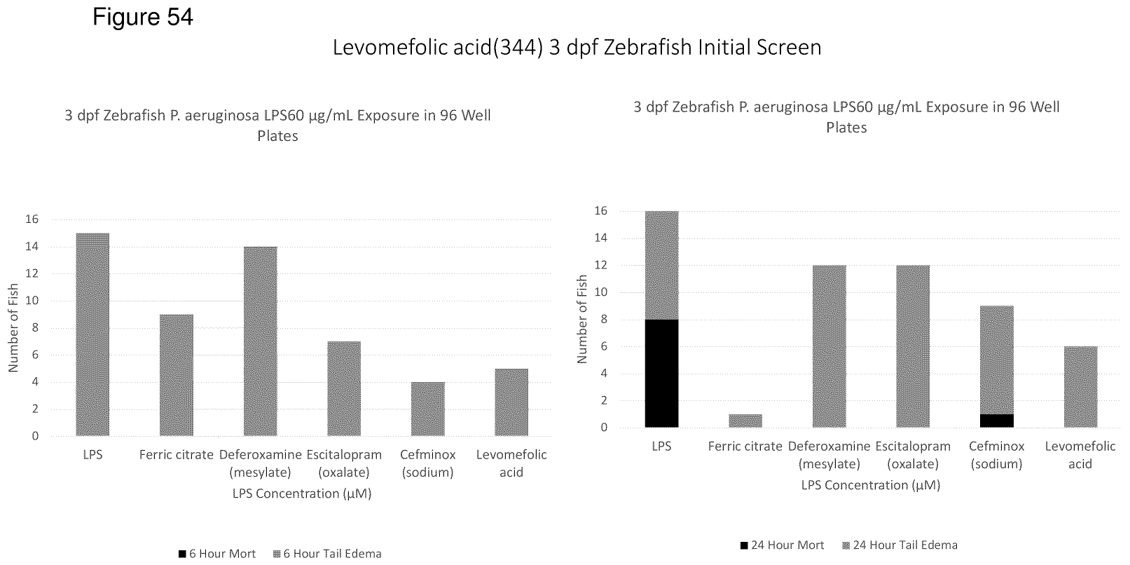

FIG. 55. Levomefolic acid (344) 5-dpf zebrafish screen under the condition of 5-dpf zebrafish P. aeruginosa LPS60 μg/mL exposure in 96 well plates.

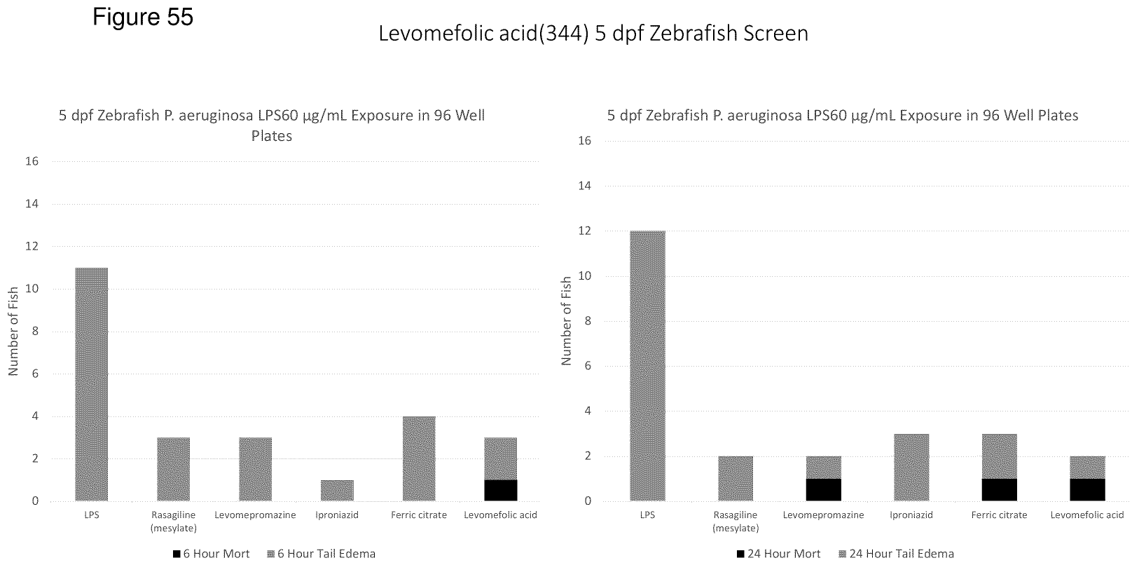

FIG. 56. Levomefolic acid (344) dose range find under the condition of 5-dpf zebrafish P. aeruginosa LPS60 μg/mL exposure in 96 well plates.

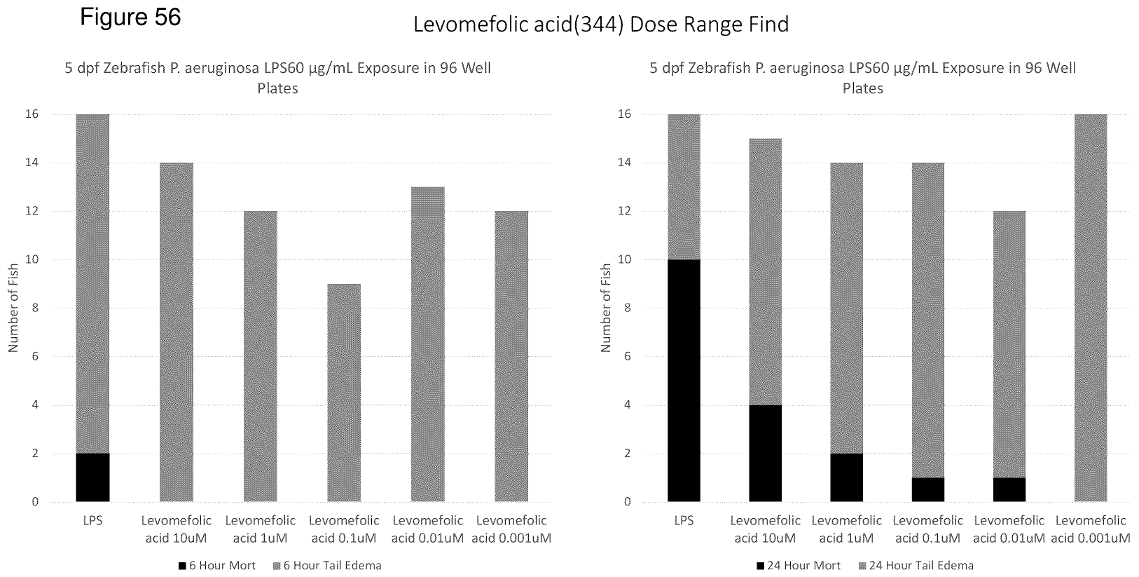

FIG. 57. Ferric citrate (340) 3-dpf zebrafish initial screen under the condition of 3-dpf zebrafish P. aeruginosa LPS60 μg/mL exposure in 96 well plates.

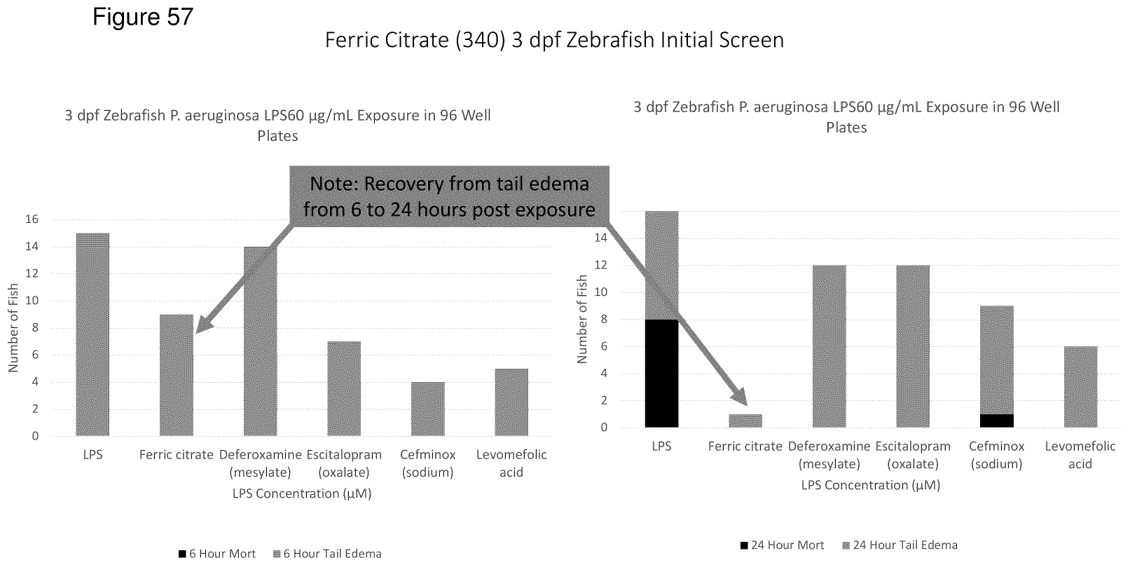

FIG. 58. Ferric citrate (340) 5-dpf zebrafish screen under the condition of 5-dpf zebrafish P. aeruginosa LPS60 μg/mL exposure in 96 well plates.

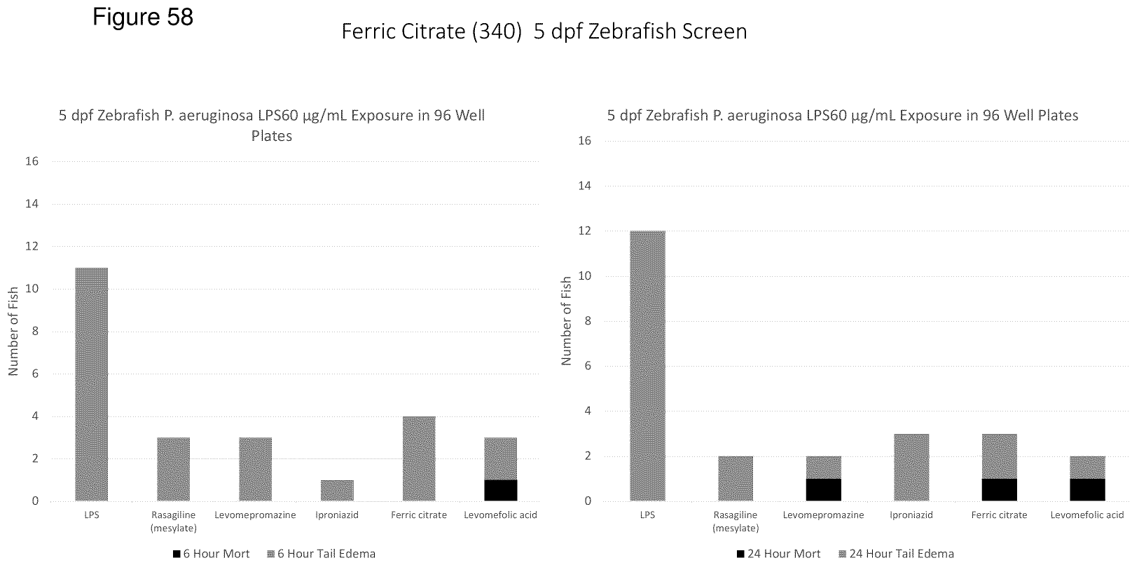

FIG. 59. Ferric citrate (340) dose range find under the condition of 5-dpf zebrafish P. aeruginosa LPS60 μg/mL exposure in 96 well plates.

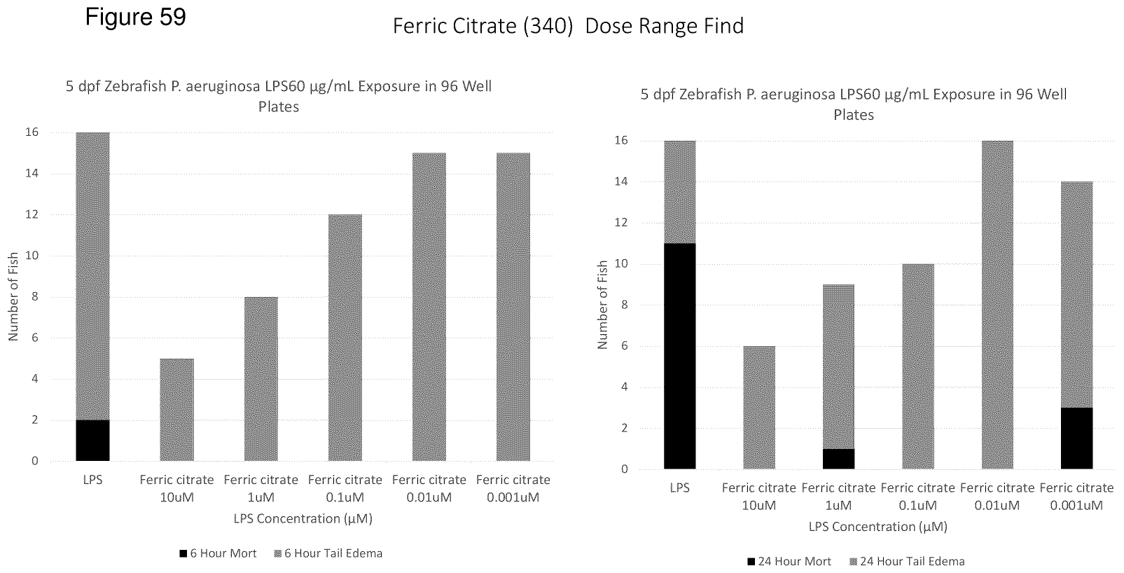

FIG. 60. Vortioxetine (376) 3-dpf zebrafish initial screen under the condition of 3-dpf zebrafish P. aeruginosa LPS60 μg/mL exposure in 96 well plates.

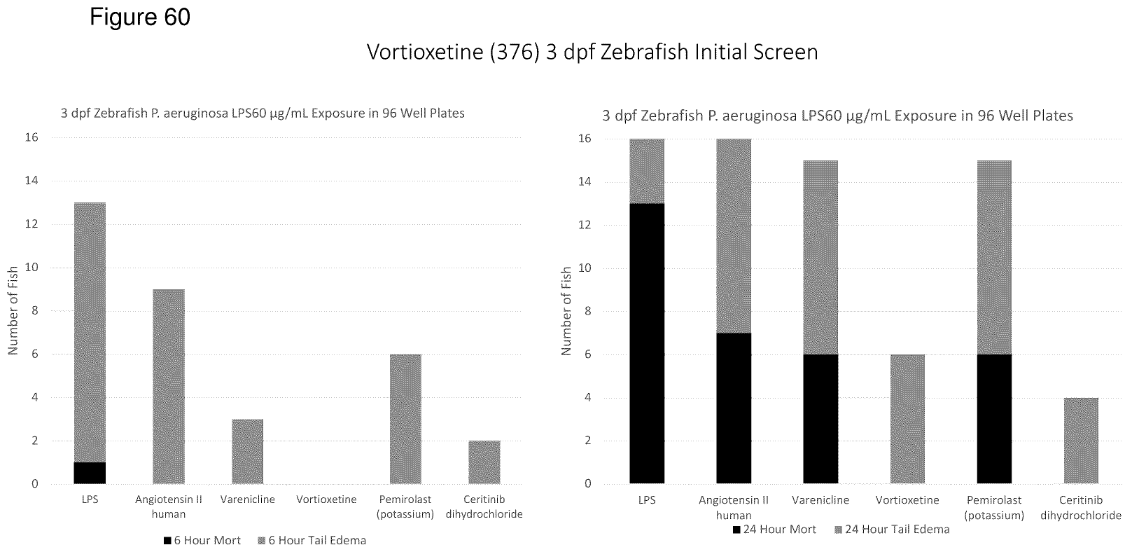

FIG. 61. Vortioxetine (376) 5-dpf zebrafish screen under the condition of 5-dpf zebrafish P. aeruginosa LPS60 μg/mL exposure in 96 well plates.

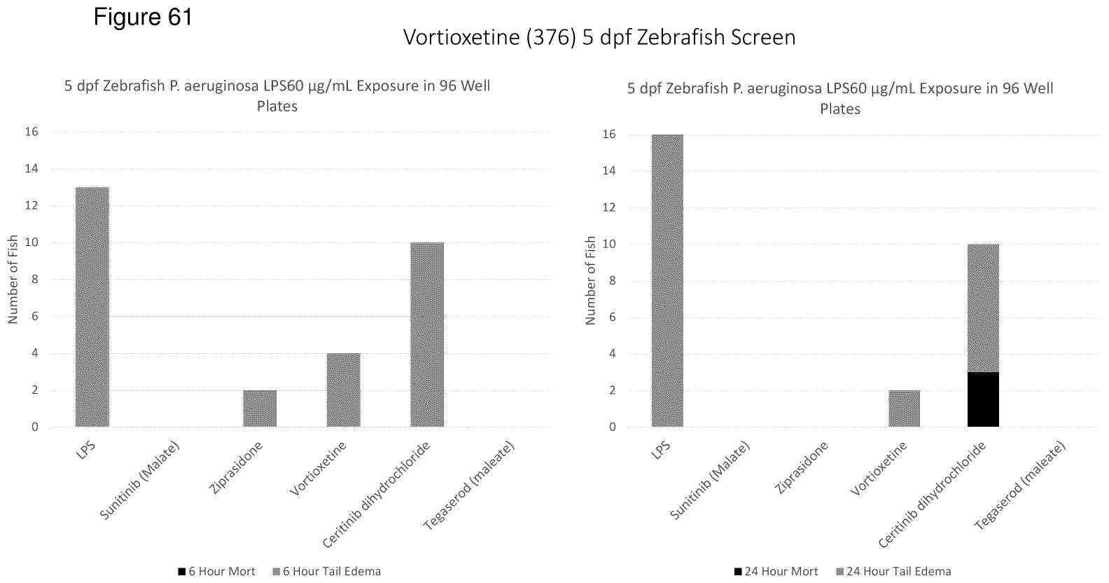

FIG. 62. Vortioxetine (376) dose range find under the condition of 5-dpf zebrafish P. aeruginosa LPS60 μg/mL exposure in 96 well plates.

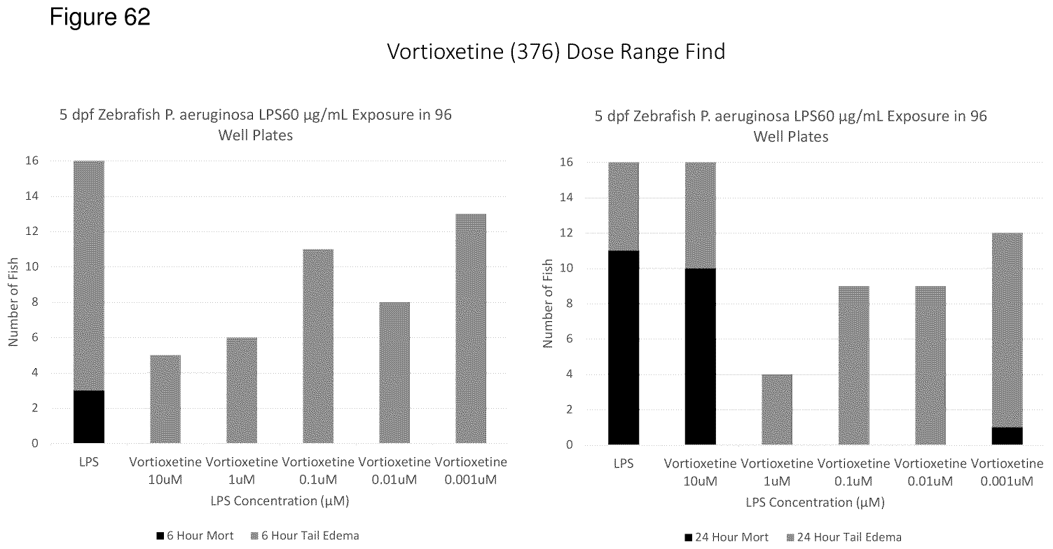

FIG. 63. Rasagiline mesylate (314) 3-dpf zebrafish initial screen under the condition of 3-dpf zebrafish P. aeruginosa LPS60 μg/mL exposure in 96 well plates.

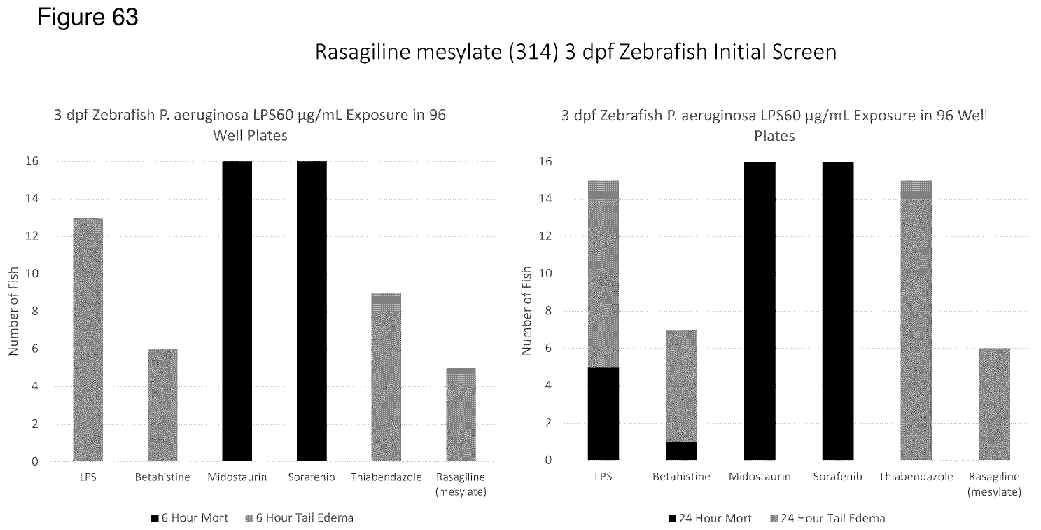

FIG. 64. Rasagiline mesylate (314) 5-dpf zebrafish screen under the condition of 5-dpf zebrafish P. aeruginosa LPS60 μg/mL exposure in 96 well plates.

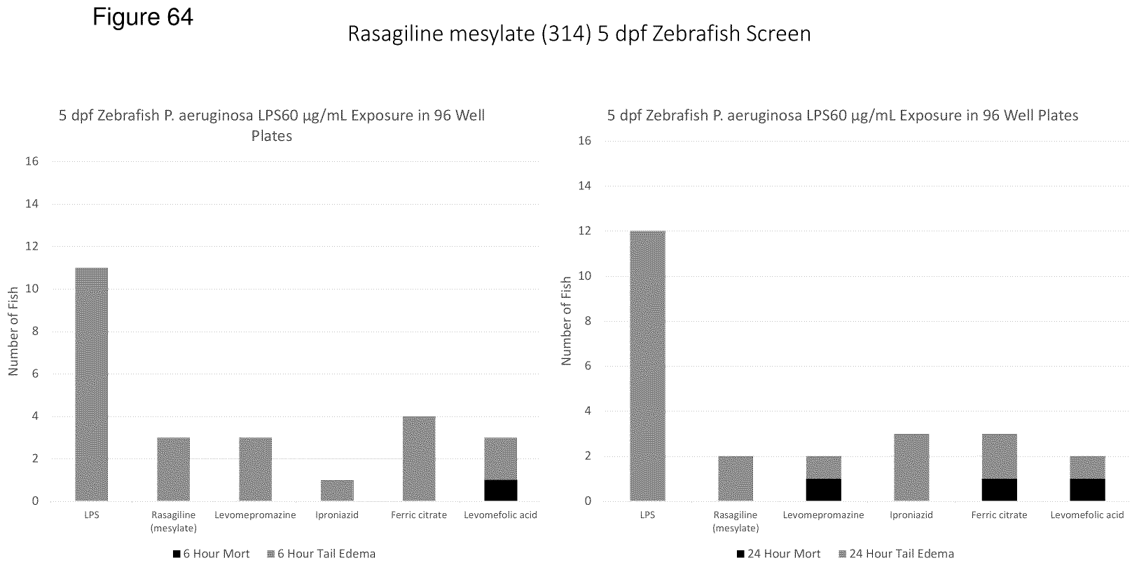

FIG. 65. Rasagiline mesylate (314) dose range find under the condition of 5-dpf zebrafish P. aeruginosa LPS60 μg/mL exposure in 96 well plates.

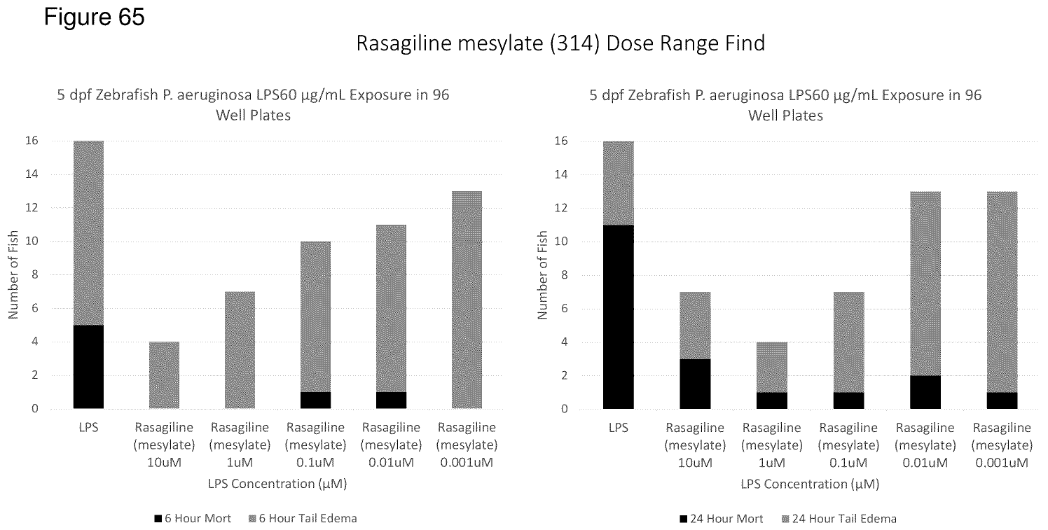

FIG. 66. Moclobemide (5) 3-dpf zebrafish initial screen under the condition of 3-dpf zebrafish P. aeruginosa LPS60 μg/mL exposure in 96 well plates.

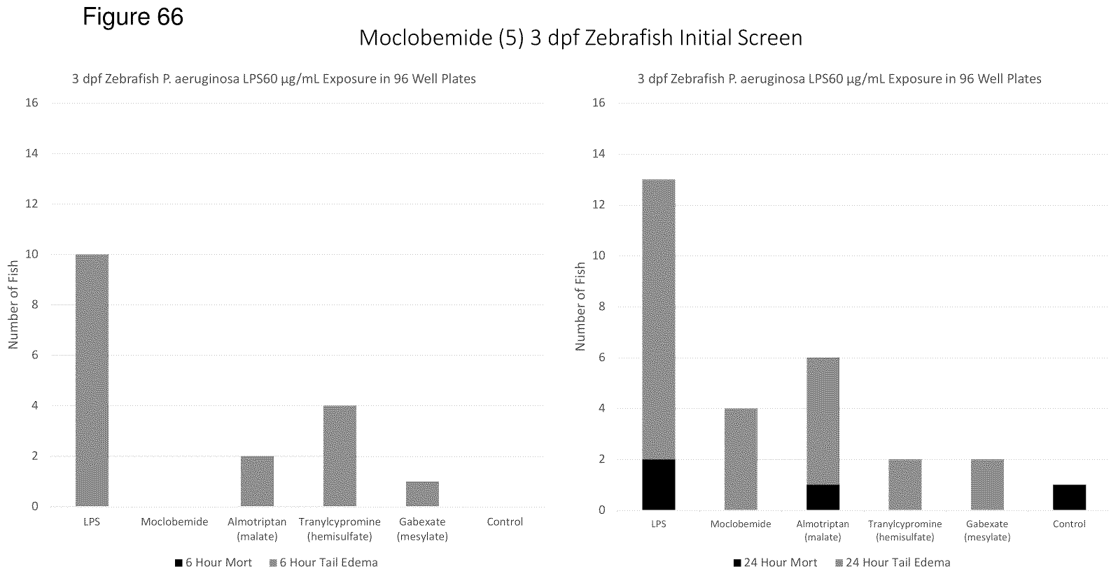

FIG. 67. Moclobemide (5) 5-dpf zebrafish screen under the condition of 5-dpf zebrafish P. aeruginosa LPS60 μg/mL exposure in 96 well plates.

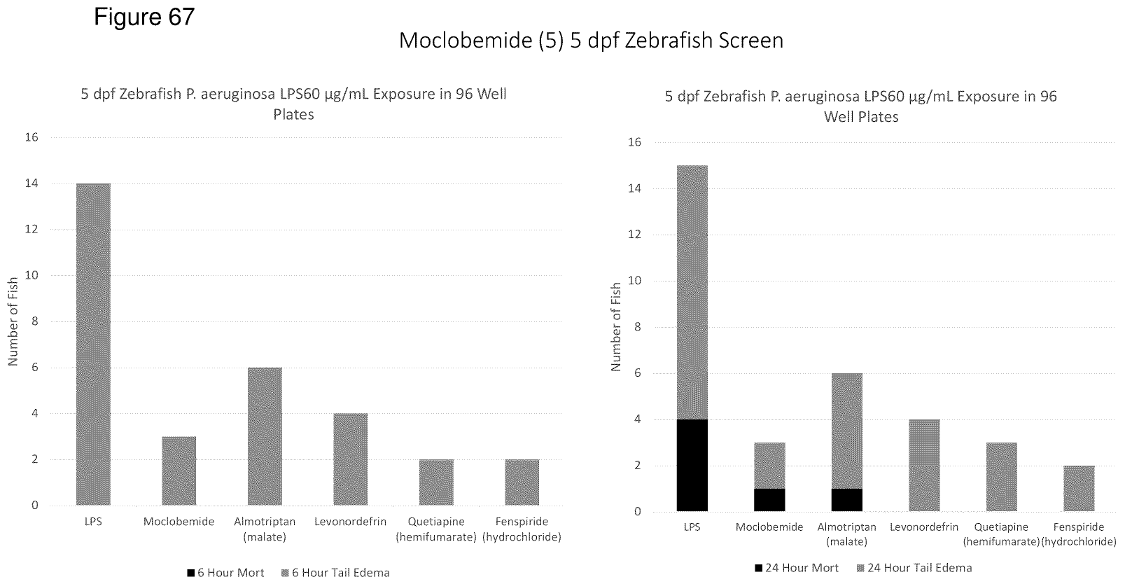

FIG. 68. Moclobemide (5) zebrafish dose range find under the condition of 5-dpf zebrafish P. aeruginosa LPS60 μg/mL exposure in 96 well plates.

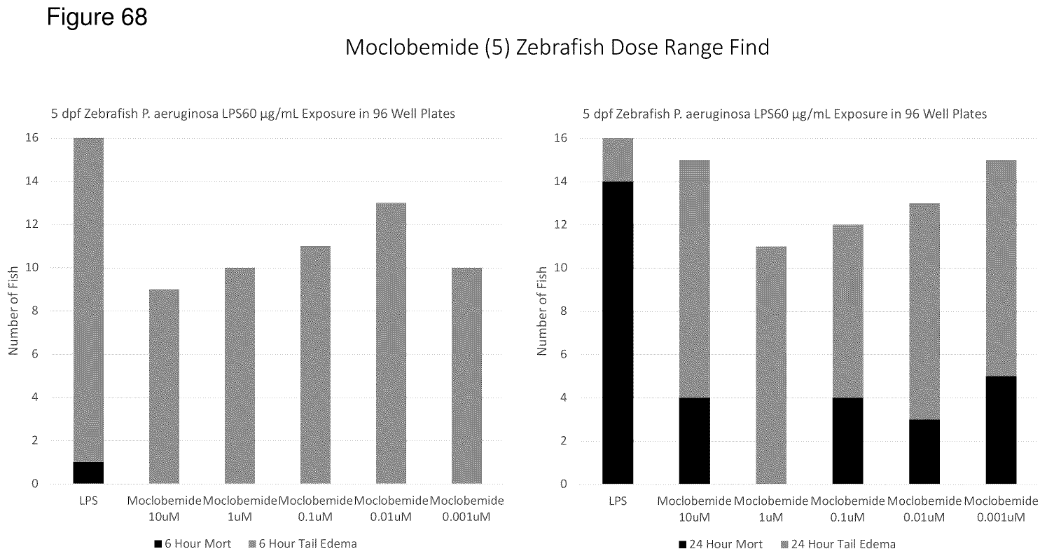

FIG. 69. Iproniazid (322) 3-dpf zebrafish initial screen under the condition of 3-dpf zebrafish P. aeruginosa LPS60 μg/mL exposure in 96 well plates.

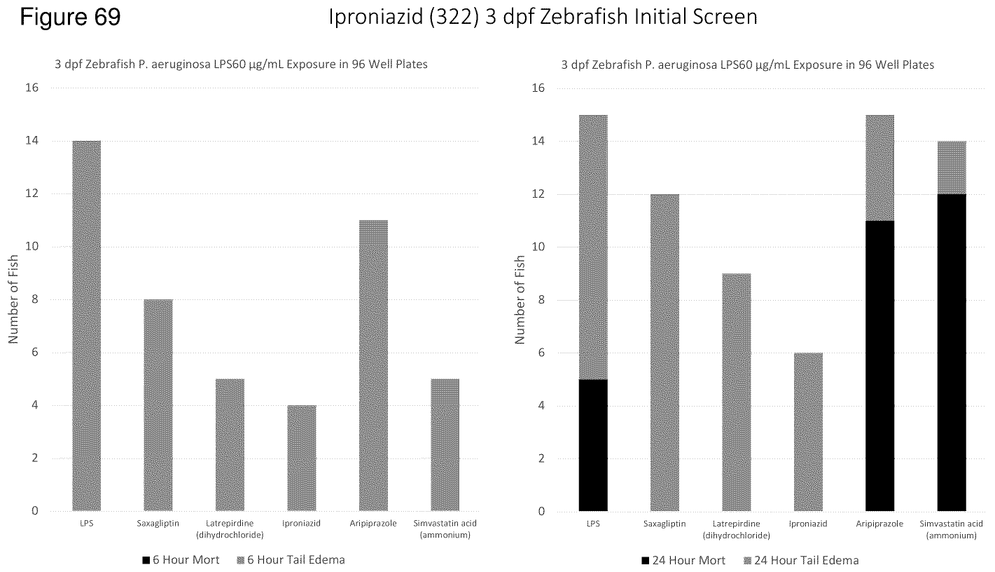

FIG. 70. Iproniazid (322) 5-dpf zebrafish screen under the condition of 5-dpf zebrafish P. aeruginosa LPS60 μg/mL exposure in 96 well plates.

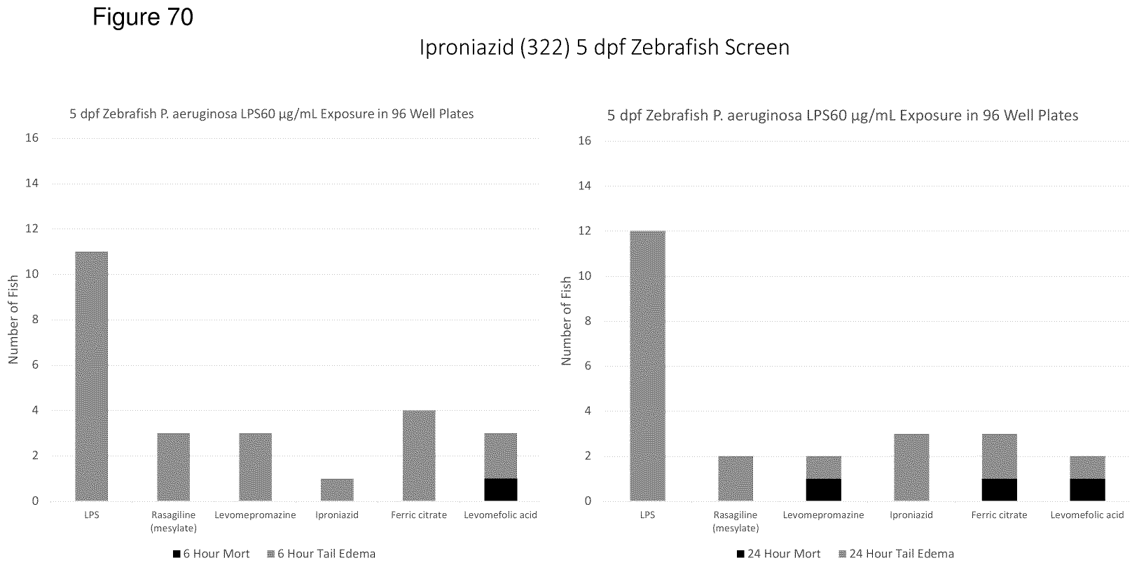

FIG. 71. Iproniazid (322) zebrafish dose range find under the condition of 5-dpf zebrafish P. aeruginosa LPS60 μg/mL exposure in 96 well plates.

FIG. 72. 6 Hour P. aeruginosa LPS exposure to 3-dpf zebrafish embryos with ROS in round bottom 96 well plates.

FIG. 73. Fluorescence image optimization and quantification Keyence image analysis software (area and brightness quantification)

FIG. 74. 6 hour P. aeruginosa LPS exposure to 3-dpf zebrafish embryos with ROS T004

FIG. 75. 24 hour P. Aeruginosa LPS exposure to 3-dpf zebrafish embryos with ROS T004

FIG. 76. ROS 24 hour evaluation with 400 M fisetin and LPS (P. aeruginosa)

FIG. 77. Fisetin 100 μM (Multiple LPS concentrations of P. aeruginosa)

FIG. 78. Fisetin 400 μM (Multiple LPS concentrations of P. aeruginosa)

FIG. 79. Fasudil 10 μM (Multiple LPS concentrations of P. aeruginosa)

FIG. 80. Fasudil 100 μM (Multiple LPS concentrations of P. aeruginosa)

FIG. 81. Dexamethasone 100 μM (P. aeruginosa LPS)

FIG. 82. Hydrocortisone 100 μM (P. aeruginosa LPS)

### DETAILED DESCRIPTION

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

As used herein, the term “about,” means plus or minus 20 percent of the recited value, so that, for example, “about 0.125” means 0.125+0.025, and “about 1.0” means 1.0±0.2.

As used herein, the terms “treatment,” “treating,” and the like, refer to obtaining a desired pharmacologic and/or physiologic effect through administering compound(s) or composition(s). “Treatment,” includes: (a) preventing, partially preventing, reversing, alleviating, reducing the likelihood of, or inhibiting the condition or disease (or symptom thereof) from occurring in a subject who is predisposed to the condition or disease but have not yet been diagnosed as having it, (b) inhibiting the condition or disease or symptom thereof, such as, arresting its development; and (c) relieving, alleviating or ameliorating the condition or disease or symptom thereof, such as, for example, causing regression of the condition or disease or symptom thereof. Treatment can include administering one or more agents, performing a procedure such as surgery or applying radiation and the like, or both. In some embodiments, treatment for sepsis may include hydration, including but not limited to normal saline, lactated ringers solution, or osmotic solutions such as albumin. Further conventional treatments that can be administered in conjunction with the enumerated agents described herein for sepsis may also include transfusion of blood products or the administration of vasopressors including but not limited to norepinephrine, epinephrine, dopamine, vasopressin, or dobutamine. Some patients with sepsis will have respiratory failure and may require ventilator assistance including but not limited to biphasic positive airway pressure or intubation and ventilation. Other agents for treating sepsis include non-steroidal anti-inflammatory agents, anti-pyretic agents, and antibiotics.

As used herein, the term “subject” or “patient” are used interchangeably to refer to a human or non-human mammal or animal. Non-human mammals include livestock animals, companion animals, laboratory animals, and non-human primates. Non-human subjects also specifically include, without limitation, chickens, horses, cows, pigs, goats, dogs, cats, guinea pigs, hamsters, mink, and rabbits. In some embodiments, a subject is a human burn patient. A “subject in need” is a subject that is at risk of developing severe inflammation, sepsis, or septic shock, or who manifests any characteristics or symptoms of severe inflammation, sepsis or septic shock.

As used herein, the term “administering” and its cognates refer to introducing an agent to a subject, and can be performed using any of the various methods or delivery systems for administering agents or pharmaceutical compositions, and any route suitable for the composition and the subject, as known to those skilled in the art. Modes of administering include, but are not limited to oral administration, parenteral administration (e.g., intravenous, subcutaneous, intramuscular or intraperitoneal injections), or local/topical administration directly into or onto a target tissue. Administration by any route or method that delivers a therapeutically effective amount of the drug or composition to the cells or tissue to which it is targeted is suitable for use with the invention. The terms “co-administration” or “co-administering” as used herein refer to the administration of a substance before, concurrently, or after the administration of another substance such that the biological effects of either substance synergistically overlap.

The term “enumerated agents” refer to drug that suppresses the activity of 5-hydroxytryptamine (5-HT, serotonin) receptors, drug that inhibits the activity of receptor tyrosine kinases (RTKs), folic acid supplement (e.g. levomefolic acid) and/or iron supplement (e.g. ferric citrate), or a drug that suppresses the activity of histamine receptors. Examples of 5-HT suppressing agents include ketanserin, tegaserod, paliperidone, paliperidone palmitate, quetiapine, ziprasidone, brexpiprazole, ritanserin, phenoxybenzamine, cyproheptadine, aripiprazole, chlorpromazine, clozapine, olanzapine, risperidone, nefazodone, and trazodone, in particular ketanserin, tegaserod, paliperidone, paliperidone palmitate, quetiapine, ziprasidone, brexpiprazole, and ritanserin. Examples of agents that suppress activity of histamine receptors includes but is not limited to mepyramine, triprolidine, cetirizine, cimetidine, ranitidine, tiotidine, thioperamide, iodophenpropit, clobenpropit, tiprolisant, proxyfan, promethazine, and pitolisant, in particular pitolisant.

As used herein, the term “combination,” with respect to administration of more than one active agent to a subject, i.e., combination therapy, refers to administration of more than one agent simultaneously or at different times. The agents can be formulated into pharmaceutical compositions that contain an agent as an active ingredient individually or pharmaceutical compositions that contain one or more agent(s). The different pharmaceutical compositions can be formulated for the same or different routes of administration. The administration of the separate pharmaceutical compositions can be accomplished at the same time, in quick succession, or separated in time by minutes, hours, days, or weeks. A combination pharmaceutical composition contains more than one active ingredient (i.e., the agent) and a pharmaceutically acceptable excipients.

As used herein, the term “effective amount” refers to an amount of an drug, agent, compound, or composition that alleviates or reduces symptoms of sever inflammation or sepsis in a subject suffering from bacterial or virus infection, and decreases mortality due to the sepsis, and/or an increases overall survival. As will be appreciated by those of ordinary skill in the art, the absolute amount of a particular agent that is effective may vary depending on such factors as the biological endpoint, the particular active agent, the target tissue, etc. Those of ordinary skill in the art will further understand that an “effective amount” may be administered in a single dose, or may be achieved by administration of multiple doses over a period of time. An effective amount of a pharmaceutical composition that contains an effective amount of one or more agents is the amount of each agent such that the overall composition is effective.

As used herein, the term “inflammation” refers to a protective response to get rid of foreign invaders (e.g., bacteria, virus) and damaged cells, which is a complex reaction mainly of blood vessels and leukocytes. Inflammation may be acute or chronic. Acute inflammation develops rapidly (typically in minutes to hours), and it lasts for a short duration (hours or a few days). Acute inflammation is characterized by redness (rubor), heat (calor), swelling (tumor), and pain (dolor) due to increased blood flow and edema. Acute inflammation is the principal way by which the innate immune system responds to microbial infections or tissue injuries. Acute inflammation begins when microbes break the epithelial barriers or when tissue is injured, which activates sentinel cells, which are present in normal tissues, such as macrophages, dendritic cells, and mast cells. Chronic inflammation is of a longer duration, and characterized by the presence of agranular leukocytes (lymphocytes and monocytes/macrophages), the proliferation of blood vessels (angiogenesis), tissue destruction, and fibrosis. Although inflammation is terminated when the causes of inflammation, i.e., foreign invaders or damaged cells/tissues, are eliminated, since it is closely intertwined with destruction of the foreign invaders and damaged cells/tissues as well as normal cells/tissues, inflammation may be detrimental to the host.

Inflammation, in particular acute inflammation, is mediated by various chemical molecules. Cell-derived mediators are usually stored in intracellular granules and rapidly released by exocytosis mechanisms (e.g., histamine from mast cells or serotonin from platelets) or are synthesized in response to a stimulus (e.g., prostaglandins, cytokines). In acute inflammation, platelets, neutrophils, monocytes/macrophages, and mast cells play key roles, but mesenchymal cells such as endothelium, smooth muscle, fibroblasts, and most epithelial cells can also secrete some of the mediators. Plasma-derived mediators (e.g., complements, kinins) are produced mainly in the liver and circulate in the blood as inactive precursors. They need to be activated, usually by a series of proteolytic cleavages, to obtain their biological properties. ((Robbins and Cotran Pathologic Basis of Diseases, Kumar, Abbas, Fausto, and Aster, 8^{th} Edition, Chapter 2; Cellular and Molecular Immunology, A. K. Abbas, A. H. Lichtman, and S. Pillai, 10th Edition, Chapter 4.)

As used herein, the term “sepsis” refers to a severe bacterial infection. In sepsis, large amounts of bacteria, especially Gram positive bacteria and endotoxin-producing Gram negative bacteria, introduced into the blood are carried through the blood from one tissue to another and cause extensive damage in wide areas of the body. The large amounts of bacteria and endotoxins in the blood stimulate the synthesis and release of large quantities of various cytokines, e.g., TNF and IL-1, increasing the levels of these cytokines in the circulating blood. High levels of cytokines cause various clinical manifestations.

As used herein, the term “septic shock” refers to a condition sometimes occurring in severe sepsis, and it is a form of distributive shock in which a widespread infection and inflammation causes cardiovascular failure and consequently other organs failure. In early stages of septic shock, only signs of bacterial infection are manifest, but as the infection becomes more severe, a large loss of plasma volume into the infected tissues takes place due to increased permeability of blood capillary walls, eventually leading to the deterioration of the cardiovascular system, which causes other organ failure, such as liver, kidney, lung and brain through ischemia, nutrient insufficiency, accumulation of lactic acid, and increase of ROS oxidative stress, among others. Examples of infection causing septic shock are peritonitis due to rupture of the gastrointestinal system, peritonitis due to female reproduction organ infection, infection resulting from wide skin infection, generalized gangrenous infection spreading to the organs via the blood, especially the liver, and kidney or urinary tract infection spreading into the blood.

As used herein, the term “shock” or “circulatory shock” refers to a life-threatening condition that occurs when the body is not getting enough blood flow, which causes insufficient supply of oxygen and nutrients to the cells/tissues/organs as well as accumulations of acids and toxic substances so as to damage the cells/tissues/organs. Shock requires immediate treatment and can get worse very rapidly. As many as 1 in 5 people in shock will die from it. The main types of shock include cardiogenic shock (due to heart problems), hypovolemic-hemorrhagic shock (due to diminished blood volume and decreases in the filling pressure of the circulation), anaphylactic shock (caused by histamine mediated allergic reaction), septic shock (due to infections), neurogenic shock (due to loss of vasomotor tone throughout the body and increased vascular capacity). In the case of hemorrhagic/hypovolemic shock, the progress of the shock can be divided into 3 stages: (1) a non-progressive stage in which full recovery is possible by normal compensatory mechanisms (baroreceptor reflexes, central nervous system ischemic response, blood vessel constriction, activation of renin/angiotensin II and antidiuretic hormone for peripheral arteriole constriction and kidney water retention, increase of epinephrine and norepinephrine by adrenal medullae for peripheral arteriole constriction) (2) a progressive stage (cardiac depression, vasomotor failure, increased capillary permeability, release of toxins by ischemic tissues such as histamine, serotonin, and tissue enzymes, accumulation of lactic acid, generalized cellular deterioration, etc.), in which, if not intervened, the shock becomes worse due to a positive feedback (i.e., vicious cycle), and (3) an irreversible stage in which although the patient is still alive, no known forms of treatment can save the life.

As used herein, the term “edema” refers to the presence of excess fluid in the tissues. Intracellular edema causes intracellular swelling, which occurs when blood flow becomes too low to supply adequate nutrients and oxygen to the cells, causing metabolism depression, Na⁺/K⁺ ATPase depression, sodium leakage into the interior of the cells, and osmosis of water into the cells. Extracellular edema occurs when excess fluid accumulates in the extracellular space. The main reason of extracellular edema is (1) abnormal leakage of fluid from the plasma into the interstitial spaces through the capillaries (excessive capillary filtration), and (2) lymphatic failure to drain the excessive interstitial space fluid back into the blood. Excessive capillary filtration is determined by the equation;

$$Filtration = {K}_{f} \times ( {P}_{c} - {P}_{if} ) - ( {π}_{c} - {π}_{if} )$$

where K_{f} (the product of the permeability and surface area of the capillaries) is the capillary filtration coefficient, P_{c} is the capillary hydrostatic pressure, P_{if} is the interstitial fluid hydrostatic pressure, π_{c} is the capillary plasma colloid osmotic pressure, and π_{if} is the interstitial fluid colloid osmotic pressure. Therefore, edema caused by fluid filtration depends on the increased capillary filtration coefficient, increased capillary hydrostatic pressure, and decreased plasma colloid osmotic pressure. (Guyton and Hall Textbook of Medical Physiology, J. Hall and M. E. Hall, 14^{th} Edition, Chapter 25)

The term “histamine” refers to an organic nitrogenous compound involved in local immune responses communication, as well as regulating physiological functions in the gut and acting as a neurotransmitter for the brain, spinal cord, and uterus. Histamine is released in almost every tissue when the tissue is damaged. Most of histamine is released from mast cells in the damaged tissues and from basophils in the blood. Histamine is a potent vasodilator, causing smooth muscle relaxation on the arterioles and, like bradykinin, increases capillary permeability, allowing fluid and small protein leakage into the tissues. Therefore, histamine increases blood flow and osmotic fluid leakage out of the circulation into the tissues, causing edema. There are 4 different types of histamine receptors: H₁ receptors are present in vascular endothelium and smooth muscles as well as nerve endings, and coupled to G_{q} protein for IP₃/DAG and intracellular calcium increase. H₁ receptors are present in gastric mucosa, cardiomyocytes, mast cells, and brain, and coupled to Gₛ for cAMP increase. H₃ receptors are in neurons and thought to decrease transmitter release from other neurons, and coupled to Gᵢ to decrease cAMP. H₄ receptors are in leukocytes, especially in eosinophils and mast cells, and coupled to Gᵢ to decrease cAMP. (Basic & clinical Pharmacology, B. G. Katzung and A. J. Trevor, 13^{th} Edition, Chapter 16). Examples of antihistamine agents are mepyramine, triprolidine, cetirizine, cimetidine, ranitidine, tiotidine, thioperamide, iodophenpropit, clobenpropit, tiprolisant, proxyfan, promethazine, and pitolisant.

The term “serotonin” (5-hydroxytryptamine, 5-HT) refers to a monoamine compound known for its biological activity as a neurotransmitter in the brain, a local hormone in the gut, and a component of blood clotting process by platelets. There are 7 families of 5-HT receptor subtypes. In tissue injury, serotonin is released from activated platelets and induces vascular smooth muscle constriction of injured blood vessels (vasoconstriction) mainly through 5-HT₂ receptor (G_{q}, IP₃/DAG and Ca²⁺ increase) to minimize blood loss, and at the same time 5-HT can activate specific receptors on vascular endothelial cells to promote changes in endothelial permeability. Examples of serotonin receptor inhibitors include ritanserin, phenoxybenzamine, cyproheptadine, aripiprazole, chlorpromazine, clozapine, olanzapine, risperidone, nefazodone, or trazodone. In the synaptic gap in the brain, secreted serotonin is reabsorbed (“reuptake”) by serotonin transporter. When neurons reabsorbed serotonin, serotonin is removed and rendered inactive. Selective serotonin reuptake inhibitors (SSRIs, e.g., citalopram, escitalopram, fluvoxamine, fluoxetine, paroxetine, sertraline, and vortioxetine), which are the most commonly prescribed antidepressants, block the reabsorption (reuptake) of serotonin into neurons. This makes more serotonin available to improve transmission of messages between neurons. SSRIs are called selective because they mainly affect serotonin, not other neurotransmitters. Another class of antidepressants, serotonin and norepinephrine reuptake inhibitors (SNRIs, e.g., duloxetine, venlafaxine, desvenlafaxine, milacipram, levomilnacipran), block the reabsorption (reuptake) of the neurotransmitters serotonin and norepinephrine at the same time in the brain.

The term, “monoamine oxidases (MAOs)” refers to a family of enzymes that catalyze the oxidation of monoamines for the breakdown of monoamines ingested in food and to inactivate monoamine neurotransmitters. MAOs belong to the protein family of flavin-containing amine oxidoreductases, and found on the outer membrane of mitochondria in most cell types. In humans there are two types of MAO: MAO-A and MAO-B. Both are found in neurons and astroglia. MAO-A is also found in the liver, pulmonary vascular endothelium, gastrointestinal tract, and placenta. MAO-B is mostly found in blood platelets. MAO-A breaks down serotonin, melatonin, norepinephrine, and epinephrine, and MAO-B breaks down phenethylamine and benzylamine. Dopamine, tyramine, and tryptamine are broken down by both types. MAO inhibitors comprise isocarboxazid, hydracarbazine, phenelzine, tranylcypromine, bifemelane, methylthioninium chloride, pirlindole, safinamide, selegiline, rasagiline, moclobemide, and iproniazid. (Shih J C, Chen K (August 2004). “Regulation of MAO-A and MAO-B gene expression”. Current Medicinal Chemistry. 11 (15): 1995-2005.)

As used herein, the term “receptor” refers to the component of a cell or organism that interacts with a natural ligand (e.g., hormones, neurotransmitters, growth factors, cytokines, etc.) or a drug and initiates the chain of events leading to the ligand's- or drug's expected effects. The receptor's affinity for a drug, which is affected by molecular size, shape, electrical charge, and functional group, determines the concentration of the drug required to exert its effect by forming a certain number of drug-receptor complexes, and the total number of receptors may limit the maximal effect of a drug.

As used herein, the term “agonist” refers to an activator of a specific receptor, which is a drug or substance that binds to a receptor inside a cell or on its surface and activate the receptor to generate the same signal as the natural ligand normally generates when it binds to the receptor.

As used herein, the term “partial agonist” refers to an agonist that, when occupying all receptors (saturation), produces a lower response than a “full agonist”, not because of its decreased affinity for binding to receptors, but because of its mixed agonist-(competitive) antagonist property.

As used herein, the term “inverse agonist” refers to a drug or substance that binds to the same receptor as an agonist but induces a pharmacological response opposite to that of the agonist.

As used herein, the term “antagonist” refers to an inhibitor of a specific receptor, which is a drug or substance that binds to the receptor but do not activate the receptor for generation of a signal, and as a result, it interferes with the ability of an agonist to activate the receptor. The antagonist binds to a receptor that will disrupt the interaction and the function of both the agonist and inverse agonist at the receptor.

The term “RNA virus” refers to a virus—other than a retrovirus—that has ribonucleic acid as its genetic material. The nucleic acid is usually single-stranded RNA, but it may be double-stranded. RNA viruses are characterized by high mutation rates compared with most DNA systems due mainly to the lack of exonuclease proofreading activity displayed by their RNA polymerases. RNA viruses mutate faster than DNA viruses, and single-stranded viruses mutate faster than double-strand virus, in which genome size correlates negatively with mutation rate. (Sanjuán R, Domingo-Calap P. Mechanisms of viral mutation. Cell Mol Life Sci. 2016 December; 73 (23): 4433-4448.). The RNA virus comprises Paramyxoviridae (parainfluenza virus, Sendai virus, measles virus, mumps virus, respiratory syncytial virus, metapneumovirus), Orthomyxoviridae (Influenza virus types A, B, C, and thogotoviruses), Coronaviridae (Corona virus, SARS virus, MERS virus), Arenaviridae (Lassa fever virus, Tacaribe virus complex (Junin and Machupo viruses), lymphocytic choriomeningitis virus), Rhabdoviridae (Rabies virus, vesicular stomatitis virus), Filoviridae (Ebola virus, Marburg virus), Bunyaviridae (California encephalitis virus, La Crossee virus, sandfly fever virus, hemorrhagic fever virus, hantavirus), Retroviridae (human T-cell leukemia virus type I and II, HIV, animal oncoviruses), Reoviridae (Rotavirus, Colorado tick fever virus), Togaviridae (rubella virus, western, eastern, and Venezuelan equine encephalitis virus, Ross River virus, Sindbis virus, Semliki Forest virus, chikungunya virus), Flaviviridae (ellow fever virus, dengue virus, St. Lous encephalitis virus, West Nile virus, hepatitis C virus), Caliciviridae (Rhinoviruses, poliovirus, echoviruses, parechoviruses, coxsackievirus, hepatitis A virus), Heparoviridae (hepatitis E virus), Astroviridae (astrovirus), and delta agent (hepatitis D virus). (Murray P R, Rosenthal K S, and Pfaller M A, Medical Microbiology, 9^{th} Edition, Chapter 36)

As used herein, the term “pharmaceutical excipient” refers to a substance that is not unacceptably toxic to the subject to which it is administered. Pharmaceutical excipients can comprise: (1) fillers or extenders including, but not limited to, as starches, lactose, sucrose, glucose, mannitol, and/or silicic acid; (2) binders including, but not limited to, carboxymethylcellulose, alginates, gelatin, polyvinyl pyrrolidone, sucrose, and/or acacia; (3) humectants including, but not limited to, glycerol; (4) disintegrating agents including, but not limited to, agar-agar, calcium carbonate, potato or tapioca starch, alginic acid, certain silicates, and sodium carbonate; (5) solution retarding agents, such as paraffin; (6) absorption accelerators including, but not limited to, quaternary ammonium compounds; (7) wetting agents including, but not limited to, cetyl alcohol and glycerol monostearate; (8) absorbents including, but not limited to, kaolin and bentonite clay; (9) lubricants including, but not limited to, talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. By way of example, a pharmaceutically acceptable carrier, additive, or excipient can include sodium citrate or calcium carbonate.

As used herein, the term “composition” refers to a pharmaceutical composition, meaning a mixture of substances suitable for administering to an individual, which includes one or more pharmaceutically active ingredients. For example, a composition may comprise a certain amount of OMV as well as suitable pharmaceutical excipients, which are considered as inert substances, i.e., they do not have any active role in therapeutics, but they can be used to support the process to produce an effective product. The composition should suit the mode of administration, which can be oral, parenteral, topical, nasal, or rectal route. An example of the composition of the present invention, if desired, can also contain minor amounts of wetting or emulsifying agents, or pH buffering agents. These compositions can take the form of solutions, suspensions, emulsion, tablets (fast dissolving, extended release (XR) or long-acting (LA), sustained release (SR), controlled release (CR), delayed release (DR), or enteric coating formulation), pills, capsules, powders, granules, and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.”

As used herein, the term “dosage form” refers to a pharmaceutical preparation in which a specific mixture of active ingredients of a drug and inactive components (excipients) are formulated in a particular shape or form to facilitated administration and accurate delivery of active ingredients, and/or to be presented in the market. Solid dosage forms include powders, granules, capsules, tablets, cachets, pills, lozenges, gummies, suppositories. Semi-solid dosage forms include ointment, creams, paste, gels, poultices. Liquid dosage forms include collodions, droughts, elixirs, emulsions, suspension, enemas, gargles, linctuses, lotion, liniments, mouth washes, nasal drop, paints, solutions, syrups. Gaseous dosage forms include aerosols, inhalations, and sprays. (https://thepharmapedia.com/pharmaceutical-dosage-form-pharmaceutics/pharmacy-notes/)

As used herein, the term “antiviral agent” refers to an agent inhibiting virus attachment to the host cell and/or proliferation inside the host cell. Antiviral agents can be administered before, after, or at the same time with the enumerated drugs here. Antiviral agents include antibodies against viral proteins as well as inhibitors of RNA-dependent RNA polymerase (RdRp) including nucleoside analogue inhibitors (NIs), which bind to the enzyme active site; and nonnucleoside analogue inhibitors (NNIs), which allosterically inhibit the enzyme activity. NI examples include pyrimidine nucleoside inhibitors (cytosine analogue inhibitors, uracil analogue inhibitors, thymine analogue inhibitors), purine nucleoside inhibitors (adenine analogue inhibitors, guanine analogue inhibitors), and miscellaneous nucleoside inhibitors.

As used herein, the term “antibiotics” refers to antimicrobial substance active against bacteria for the treatment of bacterial infections, which usually either kill or inhibit the growth of bacteria. General antibiotics can be classified into to: (1) bacterial cell wall or membrane synthesis inhibitors, e.g., penicillins and cephalosporins; (2) protein synthesis inhibitors (e.g., macrolides, tetracyclins); and (3) DNA synthesis inhibitors (e.g., fluoroquinolones gyrase inhibitors and sulfa antibiotics inhibiting bacterial folic acid synthesis). Standard or traditional antibiotic agents include, but are not limited to: (1) cell wall or membrane synthesis inhibitor antibiotics, e.g., penicillins or beta-lactam compounds: pencillins (e.g., penicillin G, penicillin V, isoxazolyl penicillins, oxacillin, cloxacillin, flucloxacillin, dicloxacillin, nafcillin, methicillin, ampicillin, amoxicillin, piperacillin, ticarcillin, carbenicillin, aminopenicillins, carboxypenicillins, ureidopenicillins, temocillin, azlocillin, mezlocillin, mecillinam); cephalosporins and cephamycin: (e.g., cefazolin, cephalexin, cephradine, cefadroxil, cefuroxime, cefaclor, cefamandole, cefonicid, cefprozil, ceforanid, cefoxitin, cefmetazole, cefotetan, cefoperazone, cefotaxime, ceftazidime, ceftriaxone, cefepime, ceftaroline fosamil, cephalothin, cephapirin, cefpodoxime, ceftibuten, cefdinir, ceftizoxime, ceftriaxone, cefepime, cefditoren, cefixime, ceftibuten, cefacetrile, cefaloglycin, cefalonium, cefaloridine, cefatrizine, cefazaflur, cefazedone, cefroxadine, ceftezole, cefuzonam, cefcapene, cefdaloxime, cefdinir, cefditoren, cefetamet pivoxil, cefmenoxime, cefteram, ceftiofur, cefoperazone, ceftazidime latamoxef, cefclidine, cefluprenam, cefoselis, cefozopran, cefpirome, cefquinome, ceftobiprole, flomoxef (or oxa-1-cephamycin), ceftobiprole); carbacephems (e.g., loracarbef); carbapenems (e.g., biapenem, doripenem, ertapenem, imipenem, imipenem-cilastatin, meropenem, tebipenem pivoxil, faropenem, panipenem/betamipron, razupenem (PTZ-601), thienpenem (thienamycin)); monobactams (e.g., aztreonam, tigemonam, nocardicin A, or tabtoxinine β-lactam); beta-lactamase inhibitors (e.g., clavulanic acid, sulbactam, tazobactam); glycopeptides (e.g., vancomycin, teicoplanin); lipoglycopeptide (e.g. oritavancin, dalbavancin and telavancin); daptomycin, fosfomycin, bacitracin, cycloseine, isoniazid; polypeptide antibiotics (e.g., polymyxin B, or polymyxin E (colistin)); (2) antibiotics targeting bacterial ribosome subunits, the 30S and the 50S subunits; dactinomycin (or actinomycin D), chloramphenicol; tetracyclines (e.g., tetracycline, chlortetracycline, doxycycline, oxytetracycline, demeclocycline, lymecycline, meclocycline, methacycline, minocycline, rolitetracycline, or tigecycline); macrolides (e.g., erythromycin, clarithromycin, azithromycin, fidaxomicin, telithromycin, carbomycin A, josamycin, kitasamycin, midecamycin/midecamycin acetate, oleandomycin, solithromycin, spiramycin, troleandomycin, tylosin, or roxithromycin, dirithromycin, roxithromycin); aminoglycosides: (e.g., streptomycin, rhodostreptomycin, kanamycin, neomycin, amikacin, gentamicin, netilmicin, tobramycin, paromycin, apramycin) apramycin, plazomicin, arbekacin, and streptomycin); lincosamides (e.g., lincomycin, pirlimycin and clindamycin); ansamycins: geldanamycin, naphthomycin, rifamycins (e.g., rifampicin (or rifampin), rifabutin, rifapentine, rifalazil, or rifaximin); quinupristin-dalfopristin, mupirocin, streptogramins (e.g., streptogramin A, streptogramin B), oxazolidinones (e.g., linezolid, tedizolid), spectinomycin; (3) DNA replication inhibitor antibiotics: fluoroquinolones and quinolones (e.g., nalidixic acid, norfloxacin, ciprofloxacin, levofloxacin, gatifloxacin, moxifloxacin, gemifloxacin, lomefloxacin, ofloxacin, pefloxacin, moxifloxacin, rosoxacin, enoxacin, fleroxacin, nadifloxacin, rufloxacin, balofloxacin, grepafloxacin, pazufloxacin, sparfloxacin, temafloxacin, tosufloxacin, clinafloxacin, garenoxacin, stifloxacin, trovafloxacin, prulifloxacin, cinoxacin, flumequine, oxolinic acid, piromidic acid, pipemidic acid); sulfonamide antibiotics (e.g., sulfacytine, sulfisoxazole, sulfamethizole, sulfadiazine, silver sulfadiazine, sulfamethoxazole, sulphapyridine, sulfadoxine, sulfathalidine, sulfacetamide sodium, mafenide, co-trimoxazole, sulfasalazine, sulfanilamides, sultiame, sulfadimethoxine; pyrimidines (e.g., trimethoprim, pyrimethamine, and more than 200 drugs; https://go.drugbank.com/categories/DBCAT000349); others (pyrazinamide, ethambutol, streptomycin, ansamitocin); and any combination thereof can be used.

### 5-HT_{2A} Receptor

### 5-HT_{2A} Receptor Location

5-HT_{2A} receptors are expressed in many tissues and organs throughout the body including the brain, gut, and cardiovascular system (CVS), although they are most extensively expressed in the central nervous system (CNS). In CVS, 5-HT_{2A} is expressed by platelets, endothelial cells, and smooth muscle cells, and functions primarily in inflammation and wound healing. For example, when the blood vessel endothelium is damaged, serotonin is released, and vasoconstriction is triggered by the activation of 5-HT_{2A} receptors on smooth muscle cells in the resistance vasculature, including coronary arteries. In the CVS, the receptor is activated by 5-HT during wound healing and inflammation to trigger platelet aggregation and vasoconstriction.

### Intracellular Cascades Activated by 5-HT_{2A}

There are three distinct biochemical signals influenced by the 5-HT_{2A} receptor: _{IP3}/DAG, arachidonic acid (AA), and 2-aracidonyl-glycerol (2AG). Several others have been documented in the literature. As the receptor is Gq-coupled, its activation typically leads to the accumulation of IP₃/DAG, which in turn leads to calcium release from the endoplasmic reticulum and activation of protein kinase C. (Pithadia A B, Jain S M. 5-Hydroxytryptamine Receptor Subtypes and their Modulators with Therapeutic Potentials. J Clin Med Res. 2009 June; 1 (2): 72-80. doi: 10.4021/jocmr2009.05.1237. Epub 2009 Jun. 21. PMID: 22505971; PMCID: PMC3318857.)

### 2. Overview

Much has been published highlighting the role of cytokines and inflammatory pathways in the initiation and progression of sepsis. Although it was anticipated that the cytokine targeted pathways would yield a high level of positive hits for candidate sepsis rescue drugs, these pathways had the lowest number of hits of the targeted pathways screened in this study. Given the lack of efficacy of known anti-inflammatory drugs evaluated, this may not be unexpected. Likewise, modification of multiple cytokines might be required to affect sepsis progression, or cytokine pathways may be downstream of the true molecular initiation events that manifest in sepsis, septic shock and death.

To identify potential sepsis drug candidates, an optimized larval zebrafish model of endotoxicity/sepsis was used to screen commercial libraries of U.S. Food and Drug Administration (FDA)-approved drugs and other active pharmaceutical ingredients (API) known to affect pathways implicated in the initiation and progression of sepsis in humans (i.e., inflammation, mitochondrial dysfunction, coagulation, and apoptosis). In this model, endotoxicity was induced in 3- and 5-day post fertilization larval zebrafish (characterized by mortality and tail fin edema (vascular leakage)) by immersion exposure to Pseudomonas aeruginosa 60 μg/mL lipopolysaccharide (LPS) for 24 hours, then screened for the rescue potential of 644 selected drugs simultaneously with LPS at 10 μM. After LPS exposure, a neurobehavioral assay (light-dark test) was performed to further evaluate rescue from endotoxicity and to determine possible off-target drug side effects.

TABLE 1
Zebrafish sepsis model endpoints to be measured after LPS
exposure in the presence and absence of test drugs
Zebrafish LPS Rapid Screening

| Endpoints | Therapeutic Target Goal |
|---|---|
| Mortality | Improved survival from LPS exposures |
| Tail Fin Edema | Therapeutic Rescue of LPS-Induced |
| | Vascular leakage |
| ROS Production | Reduction of LPS-Induced ROS Production |

Sixteen compounds were tested and demonstrated >80% rescue. Eight of the tested 16 compounds demonstrating >80% rescue with 5-dpf larvae are known to target neuromodulatory pathways. Serotonin (5-HT receptors) pathway activity, in particular involving 5-HT2a, was present as a potential target in all 8 drugs, but some also had activity within adrenergic, dopaminergic, muscarinic, and histaminergic pathways. That said, tegasorod also targets 5-HT4 receptors, brexpiprazole also has 5-HT1 activity and ziprasidone has 5-HT1, 2, 3, 6 and 7 activity. Three compounds are monoamine oxidase inhibitors and three other top hits targeted potassium voltage-gated channels, apoptosis regulation, and tyrosine kinase inhibition. Current drug indications for the top 16 identified sepsis rescue drugs include 5 antipsychotics, 4 antidepressants, and single drugs identified for Parkinson's disease, irritable bowel syndrome, anti-cancer/vascular endothelial and platelet growth factors, narcolepsy, respiratory disease, folate deficiency and iron supplementation/phosphate binder.

Of the top five rescue drugs (Table 17), only Ketanserin has been previously evaluated in a clinical trial as a potential sepsis therapy. In a 10-person sepsis clinical trial, Ketanserin improved microcirculation, but it's vasodilatory activity may have hindered its transition to the clinic for the treatment of sepsis (Vellinga et al., 2015). Ketanserin may still have utility in situations where preventive treatment can be initiated early, prior to sepsis-associated hypotension. Except for Sunitinib, all five top rescue drugs have serotonergic (5-HT) receptor activity, which may indicate a potential common pathway for sepsis treatment and/or prevention. While Tegaserod, Brexpiprazole and Ziprasidone also target 5-HT2a receptors (and others), these drugs appear to lack the hypotension liability (as observed with Ketanserin), and thus potentially offer new options for sepsis prevention and treatment.

In addition to its well-known role within the central nervous system (CNS), 5-HT is of critical importance throughout the body, with complex modulatory roles in immune function and cellular signaling (Ahern, 2011, Zhang et al., 2017, Guan et al., 2022, Huang et al., 2022). 5-HT receptors also play a critical role in the cardiovascular system and in the regulation of endothelial barrier function and signaling; disruption of these functions has been implicated in the progression of sepsis (Ramage et al. 2008, Tanaka et al. 2021, Neumann et al. 2023). Further, prior work using a cecal ligation and puncture sepsis mouse model demonstrated that 5-HT proficient mice had a significantly lower survival rate than 5-HT-deficient mice (Zhang et al 2017), indicating that 5-HT pathways are likely involved in sepsis mortality. The known 5-HT activity of the discovered top rescue drugs, coupled with their ability to reduce LPS-induced vascular damage and mortality in the zebrafish endotoxemia model of sepsis, may indicate an ability to mediate endothelial function, which may be critical for silencing the signaling process involved in the progression of sepsis.

Of the 644 drugs in the screening library, a total of 103 drugs have known 5-HT receptor activity. Of these, only Ketanserin and Granisetron have been previously evaluated in clinical trials for sepsis. The Granisetron trial failed to improve 28-day mortality over placebo in a 150-person sepsis patient study (Guan et. al 2022). Granisetron is a 5HT3 receptor antagonist which differs in receptor activity from top rescue drugs discovered through the zebrafish endotoxemia sepsis model screening efforts. Granisetron showed protection against polymicrobial sepsis-induced acute lung injury in mice (Wang et al 2019) and sepsis-induced liver damage in rats (Aboyoussef et al., 2021). In zebrafish, Granisetron showed a significant reduction in mortality but did not rescue from vascular damage (tail edema) and thus was not selected as a leading sepsis drug candidate for further development. Recruitment for a sepsis clinical trial is currently underway for Ondansetron, another 5HT3 receptor antagonist included in the drug screening library but which was not efficacious in the zebrafish endotoxicity sepsis model.

Some results from this zebrafish sepsis model drug screening effort can be compared with findings from murine clinical studies. Published mouse LPS-induced sepsis studies showed that ciprofloxacin provided significant rescue from mortality (Ogino et al. 2009). In the zebrafish endotoxemia model used here, ciprofloxacin also provided rescue from mortality, but did not elicit rescue from vascular damage. Clinical trials for sepsis using anti-inflammatory drugs to date have not been successful (Ulloa et al., 2009). Similarly, corticosteroids such as dexamethasone, methylprednisolone, and hydrocortisone showed rescue early in the zebrafish sepsis model (6-hour exposure time point) but failed to show any level of rescue by 24 hours post exposure.

Zebrafish provide a reproducible model to evaluate sepsis therapy candidates with the additional benefit of a higher throughput over mammalian models. Neuromodulatory drugs may be viable targets for sepsis therapy. Candidates targeting 5-HT receptors have modulatory roles in the CNS but can also affect the cardiovascular system. Other identified complementary and novel targets also merit further study as sepsis therapeutics. The leading drug candidates identified in this study exceeded the therapeutic potential of commonly used compounds such as anti-inflammatory drugs and warrant follow-on evaluations using mammalian models of sepsis.

As stated above, certain embodiments of the invention is based on the discovery that drugs suppressing 5-HT receptors, in particular 5-HT₂ receptor antagonists, partial agonists and inverse agonists, and other drugs such as RTK inhibitor, antihistamine, and folic acid or iron supplement possess capabilities of reducing edema and mortality rate caused by exposure to bacterial LPS. These results suggest a potential of these drugs as treatment drugs for inflammation, sepsis, and septic shock caused by bacterial infection, and it can be contemplated that the effect of the drugs can be enhanced when co-administered with antibiotics.

In addition, considering that the progressive stage of septic shock is very similar to that of other shocks such as cardiogenic shock, neurogenic shock, and anaphylactic shock, in that uncontrolled systemic edema due to increased peripheral vasodilation and vascular permeability leads to a positive feedback of circulatory system collapse, these chemical compounds, alone or as combination of each other, can also be contemplated for the application to other shock treatments. Also, these drugs can be applied to the treatment of severe inflammation caused by virus infection, in particular mutation-prone RNA virus, resulting in “cytokine storm”, and for that purpose co-administration with other antiviral agents can be considered.

### 3. Zebrafish Model

### (1) Advantages of Zebrafish Model

Zebrafish have a high degree of genetic homology with humans (Howe et al., 2013), and have been used as a model for a range of human diseases (Lieschke and Currie, 2007). For example, pathophysiological symptoms observed in septic humans, such as dysregulated inflammatory responses (cytokine storms), tachycardia, endothelial leakage, and progressive edema have been observed in zebrafish infected with bacteria (Barber et al., 2016). Both forward and reverse genetics techniques can be applied, and many mutant strains are available to confirm and validate that the responses and mechanistic pathways are correctly targeted to improve the likely translation into human conditions of disease. In addition, zebrafish larvae are transparent and small enough that multiple fish can be held in a single well of a multi-well plate, allowing for a variety of real time continuous visualization techniques.

Zebrafish have been widely used for studying host-pathogen interactions and have an innate and adaptive immune system that becomes active at about two days and three weeks post-fertilization, respectively (Philip et al., 2017). The zebrafish immune system has many similarities to mammals (van der Sar, 2004; Sullivan and Kim, 2008; Allen and Neely, 2010; Masud et al., 2017; Torraca and Mostowy, 2018; Sisodia et al., 2020; Gomes and Mostowy, 2020), and zebrafish have been used in infection models for bacterial species of particular concern to the military, e.g., Klebsiella pneumoniae (Marcoleta et al., 2018; Leber, 2019; Zhang et al., 2019) and Pseudomonas aeruginosa (Clatworthy et al., 2009; Sullivan et al., 2017; Nogaret et al., 2021).

In both mammals and zebrafish, the lipopolysaccharide (LPS) component of the cell wall in Gram negative bacteria can trigger inflammatory responses. In mammals, LPS binds to a protein complex composed of Toll-like receptor 4 (TLR4) and myeloid differentiation factor 2 (MD-2), which recognizes lipopolysaccharide (LPS) on Gram negative bacteria to induce an innate immune response, activating a Myd88-dependent NF-κB response (ref 6 in Loes et al., 2021). Dysregulation of TLR4/MD-2 activity can induce septic shock (ref 10, 11 in Loes et al., 2021). Zebrafish do not possess a direct ortholog to TLR4, but harbor three tlr4-like genes, tlr4ba, tlr4bb, and tlr4a1. An ortholog of MD-2 (ly96) in zebrafish forms a complex with TLR4a in response to LPS, activating the NF-κB signaling response (Loes et al., 2021). Moreover, other zebrafish homologs of signaling intermediates downstream of mammalian TLRs have been identified for the adaptor proteins Myd88, Mal/Tirap, Trif/Ticam1, and Sarm, and the central intermediate Traf6 (Stein et al., 2007), and van der Vaart et al. (2012) note that “triggering of the innate immune response in zebrafish embryos also leads to induction of members of the ATF, NFκB, AP-1, IRF, and STAT families of transcription factors” (ref 13, 38 in van der Vaart et al., 2012). Overall, “the main innate immune signaling pathways (kinases, adaptors in the TLR signaling pathway, interferon response factors, signal transducers and activators of transcription) are conserved in teleost fish. Whereas the components that act downstream of the receptors are highly conserved, components that are known or assumed to interact with pathogens are more divergent” (Stein et al. (2007), summarized by Novoa and Figueras, (2012)).

Therefore, it is proposed in this disclosure to establish a zebrafish model for preliminary screening of approved drugs and novel small molecules to assess their ability to prevent manifestations of sepsis in zebrafish. A long-term impact of this screening effort is identification of new roles of approved drugs (for repurposing for a sepsis indication) or novel small molecules that prevent initiation and onset of the physiological cascade leading to sepsis.

While the zebrafish endotoxemia model developed by Hsu et al. (2018) may have some advantages, it requires microinjection of lipopolysaccharide (LPS), which is not convenient for a rapid screening test. In the Philip et al. (2017), zebrafish are exposed to LPS through immersion in 96-well plates, which induces endotoxemia, with zebrafish exhibiting “the major hallmarks of human sepsis, including edema and tissue/organ damage, increased vascular permeability and vascular leakage accompanied by altered expression of cellular junction proteins, increased cytokine expression, immune cell activation and reactive oxygen species (ROS) production, reduced circulation and increased platelet aggregation” (Philip et al., 2017).

### (2) Establishing the Zebrafish Sepsis Model

In the embodiment examples, zebrafish embryo are exposed to LPS in 96-well plates at 3-dpf, and zebrafish larvae at 5-dpf; each treatment has three replicate groups with 10 larvae per group (n=30). Various LPS exposure durations and concentrations are evaluated to optimize measurement of the primary endpoints: mortality, vascular leakage (tail fin edema), and reactive oxygen species (ROS) production. ROS production is visualized with a fluorescent microscope after treatment with 2′,7′-dichlorodihydrofluorescein diacetate (DCFH-DA), a ROS indicator. Vascular leakage is determined through observation of tailfin edema (Philip et al., 2017).

Although zebrafish have been shown to be more resistant to LPS-induced endotoxemia than humans, they are sensitive to a number of different pathogen associated molecular patterns (PAMPs), including LPS from Escherichia coli 0111:B4, Pseudomonas aeruginosa, Streptococcus agalactiae, Staphylococcus aureus, and Staphylococcus epidermidis (Phillip et al. 2017; Hsu et al. 2018; Keij et al. 2021; Novoa et al. 2009). While it is not the intent of this disclosure to screen drugs against a large number of PAMPs, follow-on studies can evaluate the efficacy of discovered drugs or drug combinations against multiple PAMPs. In this disclosure, LPS from Pseudomonas aeruginosa is used for initial model validation because of its sensitivity in zebrafish as well as its prevalence in wound infection and potential for multi-drug resistance. In some embodiments, finalized LPS formulations, concentrations, and exposure durations to be used in the optimized zebrafish model are decided.

### (3) Model Testing

After establishing the zebrafish sepsis model, different durations and concentrations of LPS exposure are evaluated with the goal of obtaining the shortest duration test that produces the characteristic endotoxemia/sepsis response. Responses using small molecule compounds shown by Philip et al. are evaluated to rescue the zebrafish from mortality, ROS production, and tail fin edema, including the anti-vascular leakage compound fasudil, a drug known to rescue LPS-induced vascular leakage in murine models of experimental sepsis (Li et al., 2010).

In some embodiments, in order to validate this in-house zebrafish sepsis model, the responses of the optimized model against compounds previously demonstrated to rescue zebrafish from the effects of LPS-induced sepsis are evaluated. Potential candidate compounds include:
Fasudil. An anti-vascular leakage compound (inhibitor of the RhoA/Rho-kinase pathway) shown by Philip et al. (2017) to rescue the zebrafish from LPS-induced mortality, ROS production, and tail fin edema. Fasudil also rescued LPS-induced vascular leakage in murine models of experimental sepsis (Li et al., 2010) and reduced acute lung injury in septic rats through inhibition of the Rho/ROCK signaling pathway (Wang et al., 2018).
Shp2 (Ptpn11a) inhibitor 11a-1. This compound led to lower NF-κB levels and lower mortality in a zebrafish endotoxemia model (Hsu et al., 2018).
Corticosteroids. Hsu et al. (2018) found that the corticosteroids hydrocortisone and dexamethasone produced responses similar to 11a-1 in their zebrafish model.
Fisetin. Molagoda et al. (2021). Fisetin is a dietary flavonoid that showed inhibition of LPS-induced inflammation and reduced mortality from endotoxic shock. Possible mechanisms include cross-talk between GSK-3β/β-catenin and the NF-κB signaling pathways.
Drug mixture. Given that sepsis likely targets multiple pathways, it will be evaluated whether the combined effect of these compounds enhances the therapeutic effect.

### (4) Rapid Screening

Once established and optimized, the zebrafish sepsis model was used to screen for possible therapeutic drugs. Between 100 and 1000 drugs were selected for screening in collaboration with Walter Reed Army Institute of Research (WRAIR) medicinal chemists as well as consultation with clinical military medical experts (Military Health System critical care and infectious disease clinicians) to ensure that key sepsis pathways are targeted and that the outcomes have a high probability of translation into clinical application to prevent sepsis.

One approach is to select drugs from available drug libraries. There are a number of options for testing drug libraries. The WRAIR repository comprises a library of ˜800 k compounds, and one ˜9000 compound library contained within the repository (CIS9000) has produced several antibacterial hits, but the anti-sepsis potential for these molecules within these libraries has yet to be assessed.

Other possibilities include natural product libraries, drug repurposing libraries (FDA approved drugs that can be tested for off-label use), and general small molecule libraries. Screening could yield molecules likely to act on pathways known to be associated with sepsis, such as upregulation of NFκB, nuclear NFκB translocation and increased MyD88 expression (Philip et al., 2017).

In addition, by using global proteomic profiling and MetaCore™ pathway enrichment analysis identified a number enriched pathways in LPS exposed zebrafish. The enriched pathways identified by Hsu et al. (2018) are also explored as potential targets for drug screening.

Next, in some embodiments it was evaluated if selected drug targets known to affect pathways are implicated in the initiation and progression of sepsis in humans, including inflammation, mitochondrial dysfunction, coagulation and apoptosis. For drugs targeting inflammation expression in sepsis, Adadey et al. (2018) and Bergmann et al. (2021) identified a number of lead drug candidates associated with the following pathways:
Topoisomerase 1 inhibition
Nuclear Factor-κB (NF-κB) repression
Inhibition of pro-inflammatory cytokines and activation of anti-inflammatory cytokines
Ubiquitin-proteasome pathway inhibition
Inhibitors of macrophage Migration Inhibitory Factor (MIF)
Inhibitors of signal transduction pathways
Interleukin-10 (IL-10) Immunomodulation
Transforming Growth Factor-β (TGF-β) Immunomodulation
Thymic stromal lymphopoietin (TSLP) Immunomodulation

Dare et al. (2009) reviewed experimental treatments for mitochondrial dysfunction and identified five categories of mitochondrial therapies: substrate provision, cofactor provision, mitochondrial antioxidants, mitochondrial reactive oxygen species scavengers, and membrane stabilizers. Drug candidates from each of these categories are considered.

Huerta and Rice (2019) described the molecular and cellular pathways involved with sepsis-related coagulation and apoptosis. Although therapeutic drugs are not discussed, the identified pathways might suggest possible drug targets. Targets for coagulation might include tissue factor (activates coagulation cascade), Plasminogen activator inh-1 (controls fibrinolysis), and Protein C and anti-thrombin (anticoagulants). Apoptosis of neutrophils and lymphocytes could also be targets. It will be ensured that the targeted pathways provided the most value for translation to clinical applications.

It is recognized that while many pathways have been identified and shown promise for treating sepsis, effective treatments remain elusive. Opal et al. (2014) highlighted a long list of clinical trial failures of novel therapeutics to treat sepsis over the past couple of decades. In their review, they provided a list of 12 recommendations that might be explored to improve the likelihood of developing new drugs for sepsis. While one recommendation noted that “Preclinical animal studies are limited in value and need to be more realistic”, another recommendation stated, “Combinations of novel agents might be needed to show significant survival benefits”.

Screening of candidate drugs associated with different molecular pathways and drug libraries will identify those drugs most effective in preventing sepsis-related effects on zebrafish mortality, tail fin edema and ROS production. Then, drug mixtures in which the individual drugs are known to affect different pathways (or have unknown pathway interactions) are evaluated to determine if a therapeutic effect can be enhanced. To our knowledge, this approach has not often been attempted, as most zebrafish mixture testing to date has been done either to identify toxic effects (e.g., Ogungbemi et al., 2021) or to use fractionation techniques to identify individual constituents of a complex mixture that have the greatest therapeutic effect (e.g., Chipiti et al., 2021).

### 4. Summary of Results

Considering the facts described above, 644 FDA-approved drugs were selected from our original library based upon the pharmacological areas identified in the literature as likely being the pathways that could be affected and/or causative for the negative outcomes of sepsis. To test the effect of the drugs, 3-dpf embryos and 5-dpf larvae were challenged with 60 μg/mL P. aeruginosa LPS in combination with a single 10 μM concentration of each compound for 24 hours, and 16 lead hit compounds were identified. Although the test concentration, dose, and volume of bacterial LPS and test drugs are set based on optimization test here, they can be different for different types of LPS and different set of test drugs within a reasonable range, for example, a total volume of a test solution in a container can be 10-1000 μL, (optionally about 50-250 μL); LPS concentration can be within a range of 10-1000 μg/mL; and test drug concentration can be within a range of 1-1000 μM.

Among them, 8 compounds target serotonergic pathways, but there are other pathways in other pharmacological areas that are also showing efficacy in this model.

Some of these drugs are described in more detail.

Ketanserin (Compound 88) and ketanserin tartrate (the tartrate salt form of ketanserin, Compound 437) (Brand Name: Sufrexal)

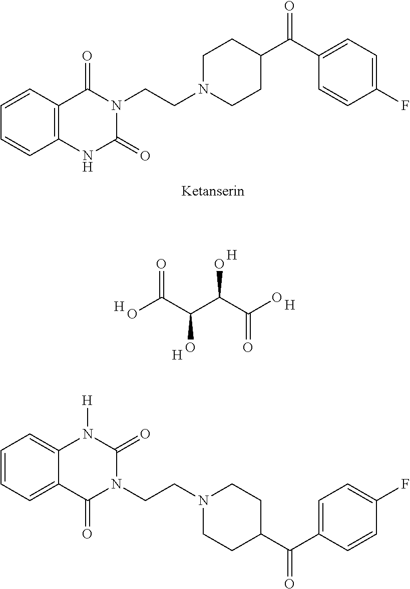

Ketanserin was first discovered by Janssen Pharmaceutica in 1980). Clinical Trials are currently ongoing (The Effect of Ketanserin on the Microcirculation in Sepsis: Leeuwarden Netherlands, https://clinicaltrials.gov/study/NCT01329887; and The effects of ketanserin on microcirculatory alterations in septic shock: An open-label pilot study, https://clinicaltrials.gov/ct2/show/study/NCT01329887). The target of ketanserin and ketanserin tartrate are 5-HT receptor, potassium channel, and autophagy. Their signaling pathways are involved with GPCR/G protein, neuronal signaling, membrane transporter/ion channel, and autophagy. The information from Drugbank indicates that ketanserin has been investigated for the treatment of septic shock, severe sepsis, and diabetic foot ulcer. Ketanserin is a selective 5-HT₂ receptor antagonist, and it also blocks hERG potassium ion channel current (IhERG) in a concentration dependent manner (IC₅₀=0.11 μM). Ketanserin significantly reduces BDNF (Brain Derived Neurotrophic Factor) protein levels in numerous brain regions (CA1 and CA3 of the hippocampus, prefrontal cortex, central amygdaloid nucleus, dorsomedial hypothalamic nucleus, dentate gyrus, shell of the nucleus accumbens and midbrain periaqueductal gray). Ketanserin can significantly reduce BDNF mRNA levels in various brain regions. Ketanserin is also a selective S2-receptor-blocking drug that decreases systolic and diastolic BP in nonpregnant patients with acute or chronic hypertension. Ketanserin is a 5-HT₂ receptor antagonist without partial agonist properties which also possesses weak alpha 1-adrenoceptor antagonistic activity, which may explain its antihypertensive mechanism of action in patients with essential hypertension. It also inhibits the effects of serotonin on platelets in cardiovascular disease, inhibits vasoconstriction caused by the amine, and when administered intravenously improves some haemorheological indices in patients with ischaemic diseases. Partly metabolized by CYP3A4 and thus could lead to high levels if administered with macrolides (erythromycin, clarithromycin, and troleandomycin, are potent inhibitors of CYP3A4)

TABLE 2
Summary of Ketanserin

| TARGET | ACTIONS | ORGANISM |
|---|---|---|
| 5-hydroxytryptamine receptor 2A | inverse agonist | Humans |

Tegaserod (maleate) (Compound 400) (Brand Name: Zelnorm™ (Alfasigma USA)

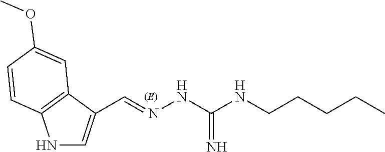 [SMILES: CCCCCNC(=N)N/N=C/c1c[nH]c2ccc(OC)cc12]

(SDZ-HTF-919) is an orally active serotonin receptor 4 (5-HT₄) agonist and a 5-HT_{2B} receptor antagonist. Tegaserod maleate has pKᵢs of 7.5, 8.4 and 7.0 for human recombinant 5-HT_{2A}, 5-HT_{2B} and 5-HT_{2C} receptors, respectively.

Zelnorm is a prescription medicine of tegaserod used to treat the symptoms of Irritable Bowel Syndrome (IBS) with Constipation. Zelnorm may be used alone or with other medications.

TABLE 3
Summary of Tegaserod

| TARGET | ACTIONS | ORGANISM |
|---|---|---|
| 5-hydroxytryptamine receptor 4 | antagonist | Humans |
| | partial agonist | |
| 5-hydroxytryptamine receptor 2C | antagonist | Humans |
| 5-hydroxytryptamine receptor 2A | antagonist | Humans |
| S-hydroxytryptamine receptor 2B | antagonist | Humans |

Paliperidone palmitate (Compound 429) (Brand Name: Invega Sustenna® (Janssen Pharmaceuticals))

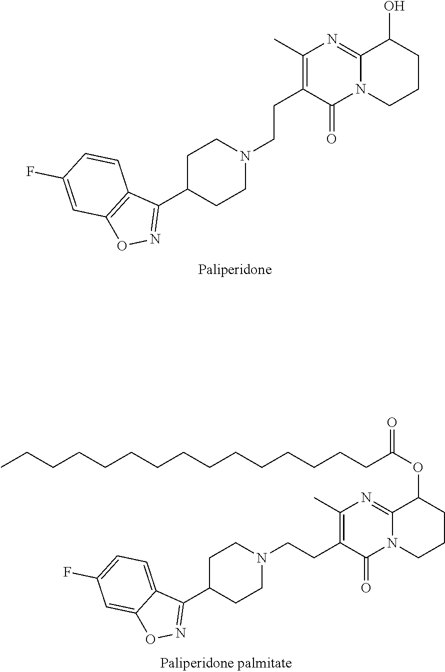 [SMILES: Cc1nc2n(c(=O)c1CCN1CCC(c3noc4cc(F)ccc34)CC1)CCCC2O ; CCCCCCCCCCCCCCCC(=O)OC1CCCn2c1nc(C)c(CCN1CCC(c3noc4cc(F)ccc34)CC1)c2=O]

Paliperidone palmitate (9-hydroxyrisperidone palmitate), an ester prodrug of paliperidone, is an atypical long-acting antipsychotic agent, and shows efficacy against schizophrenia.

Paliperidone is the primary active metabolite of risperidone. The mechanism of action is unknown, but it is likely to act via a similar pathway to risperidone. It has been proposed that the drug's therapeutic activity in schizophrenia is mediated through a combination of central dopamine Type 2 (D₂) and serotonin Type 2 (5-HT_{2A}) receptor antagonism. Paliperidone is also active as an antagonist at alpha 1 and alpha 2 adrenergic receptors and H₁ histaminergic receptors, which may explain some of the other effects of the drug.

Paliperidone was approved by the FDA for treatment of schizophrenia on Dec. 20, 2006. It is available as an extended-release tablet, a once-monthly intramuscular injection, an every-three-month intramuscular injection, and a twice-yearly gluteal injection.

TABLE 4
Summary of Paliperidone Palmitate

| TARGET | ACTIONS | ORGANISM |
|---|---|---|
| 5-hydroxytryptamine receptor 2A | antagonist | Humans |
| Dopamine D2 receptor | antagonist | Humans |
| Dopamine D4 receptor | antagonist | Humans |
| Dopamine D3 receptor | antagonist | Humans |
| 5-hydroxytryptamine receptor 2C | antagonist | Humans |
| Histamine H1 receptor | antagonist | Humans |
| Alpha-1A adrenergic receptor | antagonist | Humans |
| Alpha-1B adrenergic receptor | antagonist | Humans |
| 5-hydroxytryptamine receptor 1D | antagonist | Humans |
| Alpha-2A adrenergic receptor | antagonist | Humans |
| Alpha-2B adrenergic receptor | antagonist | Humans |
| Alpha-2C adrenergic receptor | agonist | Humans |
| 5-hydroxytryptamine receptor 1A | antagonist | Humans |
| Dopamine D1 receptor | antagonist | Humans |
| 5-hydroxytryptamine receptor 7 | Not Available | Humans |

Quetiapine (Compound 11) (Brand Name: Seroquel (AstraZeneca))

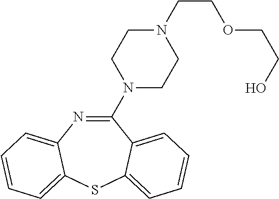 [SMILES: OCCOCCN1CCN(C2=Nc3ccccc3Sc3ccccc32)CC1]

According to information from Drugbank, quetiapine is a psychotropic agent used for the management of bipolar disorder, schizophrenia, and major depressive disorder. Initially approved by the FDA in 1997, quetiapine is a second-generation atypical antipsychotic used in schizophrenia, major depression, and bipolar disorder. Quetiapine demonstrates a high level of therapeutic efficacy and low risk of adverse effects during long-term treatment. It is well-tolerated and a suitable option for some patients with high sensitivity to other drugs, such as clozapine and olanzapine.

Quetiapine is an antagonist for D₁/D₂ receptors and 5-HT₂ receptors. It is also suggested to have the norepinephrine transporter (NET) inhibitory potential, partial agonist activity at the 5 HT_{1A} receptor, and antagonistic properties at the 5-HT_{2A} and 5-HT₇ receptors. Furthermore, its metabolite, norquetiapine has an affinity for other receptors, such as H₁ histamine receptor, 5-HT_{1E}, 5-HT_{2A}, 5-HT_{2B}, 5-HT₇ serotonergic receptors, M₁, M₃, and M₅ muscarinic receptors, and α1-adrenergic receptors. (National Institutes of Health (.gov) https://www.ncbi.nlm.nih.gov>books>NBK459145)

TABLE 5
Summary of Quetiapine

| TARGET | ACTIONS | ORGANISM |
|---|---|---|
| 5-hydroxytryptamine receptor 2A | antagonist | Humans |
| Dopamine D2 receptor | antagonist | Humans |
| 5-hydroxytryptamine receptor 1A | antagonist | Humans |
| | partial agonist | |
| 5-hydroxytryptamine receptor 1B | ligand | Humans |
| 5-hydroxytryptamine receptor 1D | ligand | Humans |
| 5-hydroxytryptamine receptor 1E | ligand | Humans |
| 5-hydroxytryptamine receptor 2C | ligand | Humans |
| 5-hydroxytryptamine receptor 3A | ligand | Humans |
| 5-hydroxytryptamine receptor 6 | antagonist | Humans |
| S-hydroxytryptamine receptor 7 | ligand | Humans |
| Dopamine D1 receptor | antagonist | Humans |
| Dopamine D5 receptor | ligand | Humans |
| Dopamine D3 receptor | ligand | Humans |
| Dopamine D4 receptor | ligand | Humans |
| Histamine H1 receptor | antagonist | Humans |
| Alpha-1 adrenergic receptors | antagonist | Humans |
| Alpha-2A adrenergic receptor | antagonist | Humans |
| Alpha-2B adrenergic receptor | antagonist | Humans |
| Alpha-2C adrenergic receptor | antagonist | Humans |
| Muscarinic acetylcholine receptor M1 | antagonist | Humans |
| Muscarinic acetylcholine receptor M2 | ligand | Humans |
| Muscarinic acetylcholine receptor M3 | antagonist | Humans |
| Muscarinic acetylcholine receptor M4 | ligand | Humans |
| Muscarinic acetylcholine receptor M5 | ligand | Humans |

Ziprasidone hydrochloride monohydrate (Compound 63) and Ziprasidone (Compound 361) (Brand Names: Geodon® (Viatris), Zeldox® (Pfizer))

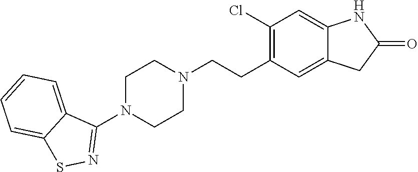 [SMILES: O=C1Cc2cc(CCN3CCN(c4nsc5ccccc45)CC3)c(Cl)cc2N1]

Ziprasidone (CP-88059) hydrochloride monohydrate is an oral antipsychotic drug classified as a “second generation” or “atypical” antipsychotic, and it is used to treat schizophrenia and bipolar disorder. Ziprasidone is and is a dopamine and 5-HT_{2A} receptor antagonist with a unique receptor binding profile, but its mechanism of action is unknown. The effects of ziprasidone are differentiated from other antispychotics based on its preference and affinity for certain receptors. Ziprasidone binds to serotonin-2A (5-HT_{2A}) and dopamine D₂ receptors in a similar fashion to other atypical antipsychotics; however, one key difference is that ziprasidone has a higher 5-HT_{2A}/D₂ receptor affinity ratio when compared to other antipsychotics such as olanzapine, quetiapine, risperidone, and aripiprazole. Ziprasidone offers enhanced modulation of mood, notable negative symptom relief, overall cognitive improvement and reduced motor dysfunction which is linked to its potent interaction with multiple serotonin receptors in addition to 5-HT_{2A}, such as 5-HT_{2C}, 5-HT_{1D}, and 5-HT_{1A} receptors in brain tissue. Ziprasidone can bind moderately to norepinephrine and serotonin reuptake sites to block monoamine transporters and prevents 5-HT and NE reuptake, which may contribute to its antidepressant and anxiolytic activity.¹ Patient's taking ziprasidone will likely experience a lower incidence of orthostatic hypotension, cognitive disturbance, sedation, weight gain, and disruption in prolactin levels since ziprasidone has a lower affinity for histamine H1, muscarinic M1, and alpha1-adrenoceptors.¹

TABLE 6
Summary of Ziprasidone

| TARGET | ACTIONS | ORGANISM |
|---|---|---|
| Dopamine D2 receptor | antagonist | Humans |
| 5-hydroxytryptamine receptor 2A | antagonist | Humans |
| 5-hydroxytryptamine receptor 1A | agonist | Humans |
| 5-hydroxytryptamine receptor 1D | antagonist | Humans |
| 5-hydroxytryptamine receptor 2C | antagonist | Humans |
| Dopamine D1 receptor | antagonist | Humans |
| Dopamine D5 receptor | antagonist | Humans |
| Dopamine D3 receptor | antagonist | Humans |
| Dopamine D4 receptor | antagonist | Humans |
| 5-hydroxytryptamine receptor 1B | antagonist | Humans |
| 5-hydroxytryptamine receptor 1E | antagonist | Humans |
| 5-hydroxytryptamine receptor 6 | antagonist | Humans |
| 5-hydroxytryptamine receptor 7 | antagonist | Humans |
| Histamine H1 receptor | antagonist | Humans |
| Alpha-1A adrenergic receptor | antagonist | Humans |
| Alpha-1B adrenergic receptor | antagonist | Humans |
| Alpha-2A adrenergic receptor | antagonist | Humans |
| Alpha-2B adrenergic receptor | antagonist | Humans |
| Alpha-2C adrenergic receptor | antagonist | Humans |
| Muscarinic acetylcholine receptor M1 | antagonist | Humans |
| Muscarinic acetylcholine receptor M2 | antagonist | Humans |
| Muscarinic acetylcholine receptor M3 | antagonist | Humans |
| Muscarinic acetylcholine receptor M4 | antagonist | Humans |
| Muscarinic acetylcholine receptor M5 | antagonist | Humans |
| 5-hydroxytryptamine 3 receptor | antagonist | Humans |
| 5-hydroxytryptamine receptor 5A | antagonist | Humans |

Brexpiprazole (Compound 45) (Brand Name: Rexulti® (Otsuka America Pharmaceutical))

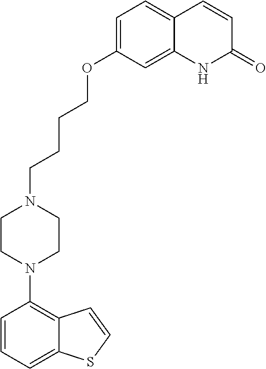 [SMILES: O=c1ccc2ccc(OCCCCN3CCN(c4cccc5sccc45)CC3)cc2[nH]1]

According to information from Drugbank, brexpiprazole is a serotonin-dopamine activity modulator used in the treatment of major depressive disorder as an adjunct and schizophrenia.

Brexpiprazole (OPC-34712), an atypical orally active antipsychotic drug, is a partial agonist of human 5-HT_{1A} and dopamine D_{2L} receptor, and an antagonist of 5-HT_{2A} receptor. Brexpiprazole has affinity (expressed as Ki) for multiple monoaminergic receptors including serotonin 5-HT_{1A} (0.12 nM), 5-HT_{2A} (0.47 nM), 5-HT_{2B} (1.9 nM), 5-HT₇ (3.7 nM), dopamine D₂ (0.30 nM), D₃ (1.1 nM), and noradrenergic α_{1A} (3.8 nM), α_{1B} (0.17 nM), α_{1D} (2.6 nM), and α_{2C} (0.59 nM) receptors. Brexpiprazole acts as apartial agonist at the 5-HT_{1A}, D₂, and D₃ receptors and as an antagonist at 5-HT_{2A}, 5-HT_{2B}, 5-HT₇, α_{1A}, α_{1B}, α_{1D}, and α² receptors. Brexpiprazole also exhibits affinity for histamine H₁ receptor (19 nM) and for muscarinic M₁ receptor (67% inhibition at 10 μM)

TABLE 7
Summary of Brexpiprazole

| TARGET | ACTIONS | ORGANISM |
|---|---|---|
| 5-hydroxytryptamine receptor 1A | agonist | Humans |
| | partial agonist | |
| Dopamine D2 receptor | agonist | Humans |
| | partial agonist | |
| 5-hydroxytryptamine receptor 2A | antagonist | Humans |
| Alpha-2C adrenergic receptor | antagonist | Humans |
| Alpha-1B adrenergic receptor | antagonist | Humans |

Repositioning of the antipsychotic drug TFP for sepsis treatment: Trifluoperazine (TFP) decreased the levels of pro-inflammatory cytokines without altering their transcription level. In LPS-induced endotoxemia and cecal content injection (CCI) models, TFP intraperitoneal administration improved survival rate. Thus, TFP was considered to inhibit the secretion of proteins through a mechanism similar to that of W7, a calmodulin inhibitor. Finally, it was confirmed that TFP treatment relieved organ damage by estimating the concentrations of aspartate transaminase (AST), alanine transaminase (ALT), and blood urea nitrogen (BUN) in the serum. Our findings were regarded as a new discovery of the function of TFP in treating sepsis patients. It is an inhibitor of calmodulin (CaM) and dopamine D2 receptor with an IC50 value of 1.2 nM for D2 receptor.

Sunitinib (Compound 97) and Sunitinib Malate (Compound 354, 621) (Brand name: Sutent (Pfizer))

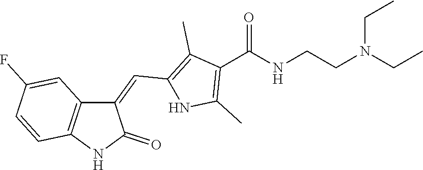 [SMILES: CCN(CC)CCNC(=O)c1c(C)[nH]c(/C=C2\C(=O)Nc3ccc(F)cc32)c1C]

Sunitinib is an oral chemotherapeutic agent for the treatment of renal cell carcinoma (RCC) and imatinib-resistant gastrointestinal stromal tumor (GIST), which was approved by the FDA on Jan. 26, 2006.

Sunitinib is a small molecule that inhibits multiple receptor tyrosine kinases (RTKs), some of which are implicated in tumor growth, pathologic angiogenesis, and metastatic progression of cancer. Sunitinib was evaluated for its inhibitory activity against a variety of kinases (>80 kinases) and was identified as an inhibitor of platelet-derived growth factor receptors (PDGFRa and PDGFRb), vascular endothelial growth factor receptors (VEGFR1, VEGFR2 and VEGFR3), stem cell factor receptor (KIT), Fms-like tyrosine kinase-3 (FLT3), colony stimulating factor receptor Type 1 (CSF-1R), and the glial cell-line derived neurotrophic factor receptor (RET). Sunitinib inhibition of the activity of these RTKs has been demonstrated in biochemical and cellular assays, and inhibition of function has been demonstrated in cell proliferation assays. The primary metabolite exhibits similar potency compared to sunitinib in biochemical and cellular assays.

TABLE 8
Summary of Sunitinib

| TARGET ACTIONS | ACTIONS | ORGANISM |
|---|---|---|
| Platelet-derived growth | inhibitor | Humans |
| factor receptor beta | | |
| Vascular endothelial growth | inhibitor | Humans |
| factor receptor 1 | | |
| Mast/stem cell growth | inhibitor | Humans |
| factor receptor Kit | | |
| Vascular endothelial growth | inhibitor | Humans |
| factor receptor 2 | | |
| Vascular endothelial growth | inhibitor | Humans |
| factor receptor 3 | | |
| Receptor-type tyrosine- | inhibitor | Humans |
| protein kinase FLT3 | | |
| Macrophage colony-stimulating | inhibitor | Humans |
| factor 1 receptor | | |
| Platelet-derived growth | inhibitor | Humans |
| factor receptor alpha | | |
| Hepatocyte growth | inhibitor | Humans |
| factor receptor | | |

Pitolisant (Compound 435) (Brand Name: Wakix (Harmony Biosciences))

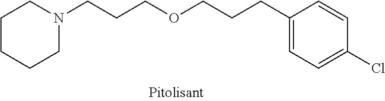 [SMILES: Clc1ccc(CCCOCCCN2CCCCC2)cc1]

Pitolisant, a nonimidazole compound, acts as a high-affinity competitive antagonist at lower nanomolar concentrations (Ki 0.16 nM) and as an inverse agonist (EC₅₀ 1.5 nM) of human histamine H₃ receptor and enhances the release of endogenous histamine over the basal level. It mediates its pharmacological action at the presynaptic level, and is used to treat type 1 or 2 narcolepsy. It is thought to bind to the antagonist binding site of the H₃ receptor, which is located within the transmembrane core just below the extracellular loops. Pitolisant displays high selectivity for H₃ receptors compared to other histamine receptor subtypes. Pitolisant also modulates acetylcholine, noradrenaline and dopamine release in the brain by increasing the levels of neurotransmitters but does not increase dopamine release in the stratal complex, including the nucleus accumbens.⁸

TABLE 9
Summary of Pitolisant

| TARGET | ACTIONS | ORGANISM |
|---|---|---|
| Histamine H3 receptor | antagonist | Humans |
| | inverse agonist | |
| Potassium voltage-gated channel | blocker | Humans |
| subfamily H member 2 | | |

Fenspiride hydrochloride (Compound 17)

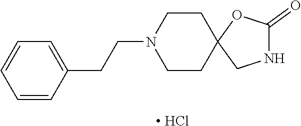 [SMILES: O=C1NCC2(CCN(CCc3ccccc3)CC2)O1 ; "· HCl"]

According to information from Drugbank, fenspiride is an oxazolidinone spiro compound used as a drug in the treatment of certain respiratory diseases. It is approved for use in Russia for the treatment of acute and chronic inflammatory diseases of ENT organs and the respiratory tract (like rhinopharyngitis, laryngitis, tracheobronchitis, otitis and sinusitis), as well as for maintenance treatment of asthma.

Levomepromazine (Compound 316) (Brand Name: Nozinan® (GL Pharma))

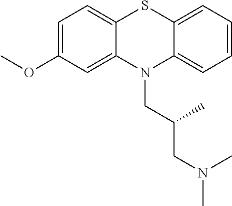

According to information from Drugbank, methotrimeprazine (levomepromazine) is a phenothiazine used in the management of psychosis, particular those of schizophrenia, and manic phases of bipolar disorder. A phenothiazine with pharmacological activity similar to that of both chlorpromazine and promethazine (antihistamine). It has the histamine-antagonist properties of the antihistamines together with central nervous system effects resembling those of chlorpromazine. (From Martindale, The Extra Pharmacopoeia, 30th ed, p 604)

TABLE 10
Summary of Levomepromazine

| TARGET | ACTIONS | ORGANISM |
|---|---|---|
| Dopamine D2 receptor | antagonist | Humans |
| Dopamine D1 receptor | antagonist | Humans |
| Dopamine D5 receptor | antagonist | Humans |
| Dopamine D3 receptor | antagonist | Humans |
| Dopamine D4 receptor | antagonist | Humans |
| 5-hydroxytryptamine receptor 2A | antagonist | Humans |
| 5-hydroxytryptamine receptor 2C | antagonist | Humans |
| Histamine H1 receptor | antagonist | Humans |
| Muscarinic acetylcholine receptor M1 | antagonist | Humans |
| Muscarinic acetylcholine receptor M2 | antagonist | Humans |
| Muscarinic acetylcholine receptor M3 | antagonist | Humans |
| Muscarinic acetylcholine receptor M4 | antagonist | Humans |
| Muscarinic acetylcholine receptor M5 | antagonist | Humans |
| Alpha-1A adrenergic receptor | antagonist | Humans |
| Alpha-1B adrenergic receptor | antagonist | Humans |
| Alpha-1D adrenergic receptor | antagonist | Humans |
| Alpha-2A adrenergic receptor | antagonist | Humans |
| Alpha-2B adrenergic receptor | antagonist | Humans |
| Alpha-2C adrenergic receptor | antagonist | Humans |

Levomefolic acid (Compound 344) (Brand Names: Beyaz 28 Day, EnBrace HR, EnLyte, Safyral 28 Day, Tydemy 28 Day, Yaz Plus)

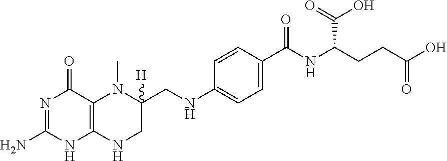 [SMILES: CN1c2c([nH]c(N)nc2=O)NCC1CNc1ccc(C(=O)N[C@@H](CCC(=O)O)C(=O)O)cc1]

According to information from Drugbank, levomefolic acid is a folate supplement used to prevent neural tube defects in pregnancy or folate deficiency. Levomefolic acid (INN) is the metabolite of folic acid (Vitamin B9) and it is a predominant active form of folate found in foods and in the blood circulation, accounting for 98% of folates in human plasma. It is transported across the membranes including the blood-brain barrier into various tissues where it plays an essential role in the DNA synthesis, cysteine cycle and regulation of homocysteine, where it methylates homocysteine and forms methionine and tetrahydrofolate (THF). Levomefolate is approved as a food additive and is designated a GRAS (generally regarded as safe) compound. It is available commercially as a crystalline form of the calcium salt (Metafolin®), which has the stability required for use as a supplement. Supplementation of levomefolic acid is desired over folic acid due to reduced potential for masking vitamin B12 deficiency symptoms. Levomefolic acid plays a critical role in methylating homocysteines into methionine by acting as a methyl donor in a reaction catalyzed by vitamine B12-dependent methionine synthase. Homocysteine must either be further metabolized via transulfuration to become cysteine, taurine, and glutathione via a B6-dependent process, or re-methylated to become methionine again. Methionine formed from remethylation of homocysteine by levomefolic acid forms the downstream metabolite S-adenosylmethionine (SAMe), which is involved in numerous biochemical methyl donation reactions, including reactions forming monoamine neurotransmitters. Studies suggest that high plasma levels of homocysteine is associated with increased incidences of arterial plaque formation.

Ferric Citrate (Compound 340), (Brand Name: Auryxia (Akebia Therapeutics)) Ferric citrate or iron (III) citrate describes any of several complexes formed upon binding any of the several conjugate bases derived from citric acid with ferric ions. Most of these complexes are orange or red-brown. They contain two or more Fe(III) centers. Ferric citrate (Iron (III) citrate), an orally active iron supplement, is an efficacious phosphate binder. Ferric citrate can be used for iron deficiency anemia and chronic kidney disease (CKD) research. Ferric citrate rescues iron deficiency and anemia in knockout mice regardless of the timing of treatment initiation, and circulating levels and bone expression of FGF23 are reduced. Ferric citrate also improves cardiac function and significantly improves survival. Ferric citrate is an efficacious and safe phosphate binder that increases iron stores and reduces intravenous iron and erythropoietin-stimulating agent use while maintaining hemoglobin. Ferric citrate can increase transferrin saturation, serum ferritin, and hemoglobin. Note: At 6 hours post exposure 9 of 16 fish had tail edemas and at 24 hours only 1 of 16 had tail edema which makes this compound unique and not observed in any other sepsis rescue compound so far.

TABLE 11
Summary of Ferric Citrate

| TARGET | ACTIONS | ORGANISM |
|---|---|---|
| Transferrin receptor protein 1 | ligand | Humans |
| Iron(3+)-hydroxamate- | binder | Escherichia coli (strain K12) |
| binding protein FhuD | | |

Vortioxetine (Compound 376) (Brand Names: Trintellix (Takeda Pharmaceuticals U.S.A))

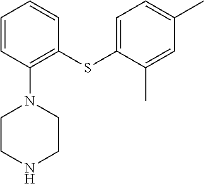 [SMILES: Cc1ccc(Sc2ccccc2N2CCNCC2)c(C)c1]

According to information from Drugbank, vortioxetine is an antidepressant medication indicated for the treatment of major depressive disorder (MDD). It is classified as a serotonin modulator and stimulator (SMS) as it has a multimodal mechanism of action towards the serotonin neurotransmitter system whereby it simultaneously modulates one or more serotonin receptors and inhibits the reuptake of serotonin. More specifically, vortioxetine acts via the following biological mechanisms: as a serotonin reuptake inhibitor (SRI) through inhibition of the serotonin transporter, as a partial agonist of the 5-HT_{1B} receptor, an agonist of 5-HT_{1A}, and an antagonist of the 5-HT₃, 5-HT_{1D}, and 5-HT₇ receptors. SMSs were developed because there are many different subtypes of serotonin receptors, however, not all of these receptors appear to be involved in the antidepressant effects of SRIs. Some serotonin receptors seem to play a relatively neutral or insignificant role in the regulation of mood, but others, such as 5-HT_{1A} autoreceptors and 5-HT₇ receptors, appear to play an oppositional role in the efficacy of SRIs in treating depression.

In addition, according to information from Takeda about Brintellix (vortioxetine), now known as Trintellix (vortioxetine), in the United States, the mechanism of the antidepressant effect of Brintellix is not fully understood. It is an inhibitor of serotonin (5-HT) reuptake and that is thought to be a mechanism of its action. It is also an agonist at 5-HT_{1A} receptors, a partial agonist at 5-HT_{1B} receptors and an antagonist at 5-HT₃, 5-HT_{1D} and 5-HT₇ receptors. The contribution of each of these activities to Brintellix's antidepressant effect has not been established. It is considered to be the first and only compound with this combination of pharmacodynamic activity. The clinical relevance of this is unknown. Brintellix was discovered by Lundbeck researchers in Copenhagen, Denmark. The clinical trial program in the U.S. was conducted jointly by Lundbeck and Takeda, and Takeda holds the new drug application for the U.S. market. Brintellix is a trademark of H. Lundbeck A/S and is used under license by Takeda Pharmaceuticals U.S.A., Inc.

TABLE 12
Summary of Vortioxetine

| TARGET | ACTIONS | ORGANISM |
|---|---|---|
| Sodium-dependent serotonin transporter | inhibitor | Humans |
| 5-hydroxytryptamine receptor 3A | antagonist | Humans |
| 5-hydroxytryptamine receptor 7 | antagonist | Humans |
| 5-hydroxytryptamine receptor 1B | partial agonist | Humans |
| 5-hydroxytryptamine receptor 1A | agonist | Humans |
| Beta-1 adrenergic receptor | ligand | Humans |
| Sodium-dependent noradrenaline | blocker | Humans |
| transporter | | |

Rasagiline mesylate (Compound 314) (Brand Name: Azilect® (Teva Pharmaceuticals))

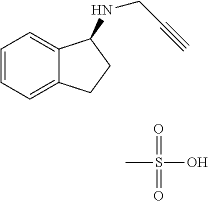

According to information from Drugbank, rasagiline is an irreversible used for the symptomatic management of idiopathic Parkinson's disease as initial monotherapy and as adjunct therapy to levodopa. Rasagiline is a propargylamine and an irreversible inhibitor of monoamine oxidase (MAO). MAO, a flavin-containing enzyme, regulates the metabolic degradation of catecholamines and serotonin in the CNS and peripheral tissues. It is classified into two major molecular species, A and B, and is localized in mitochondrial membranes throughout the body in nerve terminals, brain, liver and intestinal mucosa. MAO-A is found predominantly in the GI tract and liver, and regulates the metabolic degradation of circulating catecholamines and dietary amines. MAO-B is the major form in the human brain and is responsible for the regulation of the metabolic degradation of dopamine and phenylethylamine. In ex vivo animal studies in brain, liver and intestinal tissues rasagiline was shown to be a potent, selective, and irreversible monoamine oxidase type B (MAO-B) inhibitor. At the recommended therapeutic doses, rasagiline was also shown to be a potent and irreversible inhibitor of MAO-B in platelets. The selectivity of rasagiline for inhibiting only MAO-B (and not MAO-A) in humans and the sensitivity to tyramine during rasagiline treatment at any dose has not been sufficiently characterized to avoid restriction of dietary tyramine and amines contained in medications.

The precise mechanisms of action of rasagiline are unknown. One mechanism is believed to be related to its MAO-B inhibitory activity, which causes an increase in extracellular levels of dopamine in the striatum. The elevated dopamine level and subsequent increased dopaminergic activity are likely to mediate rasagiline's beneficial effects seen in models of dopaminergic motor dysfunction.

TABLE 13
Summary of Rasagiline

| TARGET | ACTIONS | ORGANISM |
|---|---|---|
| Amine oxidase [flavin- | inhibitor | Humans |
| containing] B | | |
| Apoptosis regulator Bcl-2 | activator | Humans |

Moclobemide (Compound 5) Brand Name: Manerix (Mylan IRE Healthcare Limited)

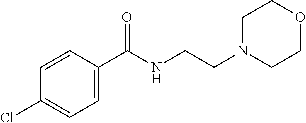 [SMILES: O=C(NCCN1CCOCC1)c1ccc(Cl)cc1]

According to information from Drugbank: A reversible monoamine oxidase inhibitor (MAOI) selective for isoform A (RIMA) used to treat major depressive disorder. Most meta-analyses and most studies indicate that in the acute management of depression, moclobemide is more efficacious than placebo medication and similarly efficacious as tricyclic antidepressants (TCA) or selective serotonin reuptake inhibitors (SSRIs). Due to negligible anticholinergic and antihistaminic actions, moclobemide has been better tolerated than tri- or heterocyclic antidepressants 1.

TABLE 14
Summary of Moclobemide

| TARGET | ACTIONS | ORGANISM |
|---|---|---|
| Amine oxidase [flavin- | antagonist | Humans |
| containing] A | inhibitor | |
| Monoamine oxidase | antagonist | Humans |
| Amine oxidase [flavin- | antagonist | Humans |
| containing] B | | |

Iproniazid (Compound 322)

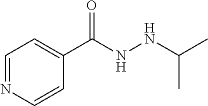

According to information from Drugbank, iproniazid is a non-selective, irreversible monoamine oxidase inhibitor (MAOI) of the hydrazine class that was developed as the first anti-depressant. (originally intended to treat tuberculosis)

### 6. Embodiments of the Invention

It is the objective of this disclosure to provide methods of using the drugs described above for the prevention of severe inflammation and sepsis from developing into septic shock and the treatment of the septic shock at its late stage, by reducing systemic edema with those drugs. Since the clinical symptoms of most circulatory shocks are very similar at their late stage, these methods may be applicable to the treatment of systemic edema occurring in cardiogenic, neurogenic and anaphylactic shock. Further, these methods may be applied to the treatment of severe inflammation due to virus infection causing “cytokine storm” and consequently circulatory shock.

### A. Targeting 5-HT_{2A} Receptors

Ketanserin, tegaserod, paliperidone palmitate, quetiapine, ziprasidone and brexpiprazole suppress 5-HT₂ (serotonin receptor) activity. Serotonin, which is an amine neurotransmitter like dopamine, norepinephrine, epinephrine, is known as vasoconstrictor, but is also reported to increase vascular permeability. So, these drugs may cause vasodilation, which is favorable condition for edema, but also cause decrease of vascular permeability and plasma leakage, which is unfavorable condition for edema. Based on the results presented in this disclosure showing the reducing effect of those drugs on edema, it can be guessed that these drugs may have more important role in vascular permeability reduction than reversing serotonin's vasoconstriction effect, which can be overcome by other vasodilatory cytokines released under infection and inflammation. In this disclosure is provided a method of preventing or treating severe inflammation, sepsis or shock by administering a drug or combination of the drug that suppresses the activity of 5-HT receptors, to a subject in need of such treatment, wherein the drug can be an antagonist, a partial agonist, or an inverse agonist of 5-HT receptors.

### B. Targeting Receptor Tyrosine Kinases

Sunitinib (and sunitinib malate) is an RTK inhibitor inhibiting PDGF and VEGF receptors and other cell growth-related receptors. The activation of VEGF receptors (and PDGF receptors and the receptors of other factors described above) is known to activate a small G protein, Rho, and its downstream signaling pathway via Rho-associated kinase (Rho-kinase/ROCK/ROK), which inhibits actin fiber assembly and induces cytoskeleton rearrangement (Shimizu A, Zankov D P, Kurokawa-Seo M, Ogita H. Vascular Endothelial Growth Factor-A Exerts Diverse Cellular Effects via Small G Proteins, Rho and Rap. Int J Mol Sci. 2018 Apr. 16; 19 (4): 1203), which makes the endothelial cells round to generate a gap between the endothelial cells, which increases vascular permeability; and thus RTK inhibitors, in particular sunitinib and sunitinib malate, may contribute to reducing vascular permeability and edema. Therefore, this disclosure provides another method of preventing or treating severe inflammation, sepsis or shock; a method of administering a drug or combination of the drug that inhibits the activity of receptor tyrosine kinases (RTKs), to a subject in need of such treatment. Further, inhibitors of RTK can be co-administered with 5-HT receptor antagonists to generate synergistic effect on reducing vascular permeability and edema.

### C. Dietary Supplement

In this disclosure, two kinds of dietary supplements, folic acid and iron supplements, reduced zebrafish tail fin edema. Although the action mechanisms are not certain, it can be guessed that these supplements can improve mitochondrial metabolisms in the damaged cells due to infection, inflammation, or hypoxia. Therefore, this disclosure provides another method of preventing or treating severe inflammation, sepsis or shock; a method of administering a dietary supplement or combination of the dietary supplement to a subject in need of such treatment, wherein the dietary supplement is folic acid supplement, levomefolic acid, and/or iron supplement, ferric citrate. Further, such dietary supplements can be co-administered with 5-HT receptor antagonists to generate synergistic effect on reducing vascular permeability and edema, with or without an RTK inhibitor.

### D. Targeting Histamine Receptors

Pitolisant is a histamine receptor H₃ antagonist. Since histamine is a vasodilator and increases vascular permeability, suppressing histamine receptors can decrease plasma leakage and edema. Therefore, this disclosure provides another method of preventing or treating severe inflammation, sepsis or shock; a method of administering a drug or combination of the drug that suppresses the activity of histamine receptors to a subject in need of such treatment, wherein the drug can be an antagonist, a partial agonist, or an inverse agonist of histamine receptors, and wherein the histamine receptor can be H₁, H₂, H₃, or H₄. Antihistamines may be co-administered with folic acid and iron supplements as well as RTK inhibitors to enhance its edema reducing effect.

### E. Use of Fenspiride Hydrochloride and Levomepromazine in Order to Reduce Edema

Fenspiride is known as an alpha-1 norepinephrine receptor blocker and to induce vasodilation, but it is also known to inhibit histamine receptor H₁ (Jankowski R. Les inflammations ORL et l′intérêt du fenspiride [ENT inflammation and importance of fenspiride]. Presse Med. 2002 September; 31 Spec No 1: HS7-10. French. PMID: 12378970), which may cause vasoconstriction while acting as a bronchodilator like many antihistamines. Levomepromazine is a “dirty drug” that exerts its effects by blocking a variety of receptors, including adrenergic receptors, dopamine receptors, histamine receptors, muscarinic acetylcholine receptors and serotonin receptors.

Generally, for some neurotransmitters, the same neurotransmitter can induce very different effects on different tissues/organs, depending on the receptor subtypes distributed in the cells of the tissue or organ. For example, catecholamines, norepinephrine and epinephrine, bind alpha-1 adrenoreceptors in vascular smooth muscles surrounding arterioles and veins to induce vasoconstriction, beta-1 adrenoreceptors in the cardiomyocytes of the heart to increase heart contractility, and beta-2 adrenoceptors in smooth muscles surrounding the respiratory tract to induce bronchodilation. In the case of dopamine, low doses cause vasodilation and decrease systemic blood pressure, whereas high doses cause vasoconstriction and increase systemic blood pressure, depending on the receptor subtypes and their affinity for the ligand. Likewise, the location of histamine receptor subtypes may show different activity, e.g., vasodilation and bronchoconstriction at the same time. Further, these neurotransmitters are the neurotransmitters released from CNS and PNS neurons, and thus one neurotransmitter may act to enhance or suppress the release of other neurotransmitters directly or indirectly, which makes the prediction of their effect on vasoconstriction/vasodilation and systemic edema more complicated, especially in such a case as sepsis or shock where various factors are network-wise connected. Thus, real experiments, at least in vivo model, are the way to decide their applicability for clinical use.

Interestingly, although the action mechanisms of fenspiride and levomepromazine are not clearly identified, both drugs showed reducing effect on sepsis symptoms in this zebrafish model. Therefore, this disclosure provides another method of preventing or treating severe inflammation, sepsis or shock; a method of administering fenspiride or levomepromazine to a subject in need of such treatment. These drugs may be co-administered with folic acid and iron supplements as well as RTK inhibitors to enhance their edema-reducing effect. They may also be co-administered with antihistamine pitolisant to generate synergistic effect for the alleviation of sepsis symptoms.

### F. Targeting Serotonin Reuptake or Degradation to Increase Serotonin Level

Serotonin reuptake inhibitors (SRIs), SNRI and SSRI are drugs increasing available serotonin molecules by blocking serotonin reuptake. In this disclosure, vortioxetine is presented as an example. MAO inhibition can also increase serotonin level by inhibiting monoamine oxidation and degradation. In this disclosure, rasagiline, moclobemide, and iproniazid are presented as examples of MAO inhibitors. It is demonstrated in this disclosure that those drugs reduced tail fin edema, indicating their usefulness in sepsis treatment. Therefore, it can be suggested to administer a selective serotonin reuptake inhibitor (SSRI), serotonin-norepinephrine reuptake inhibitors (SNRI), or monoamine oxidase inhibitor (MAOI) for preventing or treating severe inflammation, sepsis or shock.

### G. Pharmaceutical Compositions, Dosage Forms, and Routes of Administration

In preferred method embodiments, the compounds described herein are formulated and are administered as one or more pharmaceutical compositions that include a pharmaceutically acceptable excipients and one or more pharmaceutical active ingredient, including one or more of the inventive compounds described herein, and optionally including one or more of the inventive compounds described herein with an additional agent. A pharmaceutically acceptable excipient refers to any convenient compound or group of compounds that is not toxic to the subject and that does not destroy or significantly diminish the pharmacological activity of the enumerated agent(s) with which it is formulated. Such pharmaceutically acceptable carriers or vehicles encompass any of the standard pharmaceutically accepted solid, liquid, or gaseous carriers known in the art, such as those discussed in the art.

A suitable excipient depends on the route of administration contemplated for the pharmaceutical composition. Routes of administration are determined by the person of skill according to convenience, the health and condition of the subject to be treated, and the location and stage of the condition to be treated, however the preferred route of administration for the methods of this invention is oral route or intravenous, either by injection or infusion, including bolus injection, or intermittent or continuous infusion, e.g., using an infusion pump, etc. Other preferred routes of administration include direct injection into a particular area in need of treatment, such as an area of infection or autoimmune inflammation, injection into a specific blood vessel that supplies or is located at least in part within an area of infection or autoimmune inflammation of tissues or organ to be treated, mucosal administration with a mucoadhesive carrier system, intraarterial injection, intrathecal injection, subcutaneous injection, intramuscular injection, and the like.

For oral administration, the dosage form is provided in a solution, suspension, emulsion, elixir, syrup, paste, tablet (solid, chewable or lozenges), capsule, granule, powder, premix, and medicated block, comprising the chemical compound of active ingredient and pharmaceutically suitable excipients.

For intravenous administration, a liquid or semi-liquid carrier is most often used, including a solution or suspension. The forms which the pharmaceutical composition can take can include, but are not limited to: liquids, powders or granules for dilution, solutions, suspensions, emulsions, dispersions, lipid vesicles, oils, gels, and the like, for example aqueous solutions (e.g., physiological saline solutions, phosphate-buffered saline solutions, Ringer's, sodium acetate or potassium acetate solution, 5% dextrose, and the like), oil-in-water or water-in-oil emulsions. The carrier also can contain one or more of ethanol, glycerol, propylene glycol, water, a carbohydrate (e.g., glucose, sucrose, lactose), dextrans, amino acids (e.g., glycine), polyols (e.g., mannitol, a diluent, a filler, a bulking agent, a solvent, a tonicity modifying agent, a buffer, a pH-modifying agent, a surfactant (e.g., Tween-80™, Pluronic-F108/F68™, deoxycholic acid, phosphatidylcholine), a preservative, an antioxidant, an emulsifier, a chelating agent, an antimicrobial (such as an antibacterial, antifungal, or bacteriostatic compound), agent(s) to produce delayed absorption and/or any other additional compound or material, as desired. Persons of skill in the art are well aware of such compounds and can select any such excipients which are convenient and known in the art. One of ordinary skill in the art will be aware of numerous physiologically acceptable compounds that may be included in a pharmaceutical composition.

The compositions for injection are sterile, acceptably free of endotoxin, and are sufficiently fluid for easy use in a syringe. In addition, the composition should be stable under the conditions of manufacture and storage. The pharmaceutical compositions optionally are contained in a package or kit. Packages can include multiple dose vials, ampoules, pre-filled syringes, infusion bags, boxes, bottles, and the like, and may include instructions for use.

In preferred embodiments, pharmaceutical compositions are sterile solutions prepared by incorporating one or more of the active compounds in the required amount in an appropriate solvent, optionally with one or a combination of ingredients such as buffers (e.g., acetates, citrates, lactates or phosphates), agents for the adjustment of tonicity (e.g., sodium chloride or dextrose), antibacterial agents (e.g., benzyl alcohol or methyl parabens), antioxidants (e.g., ascorbic acid, glutathione, or sodium bisulfate), chelating agents (e.g., EDTA), and other suitable ingredients etc., as desired, followed by filter-based sterilization.

Pharmaceutical compositions can be formulated to contain each of the compounds alone, or in combination of at least 2 compounds described herein, optionally also including one or more additional treatment agents. Supplementary active compounds, e.g., compounds independently useful for treating a subject suffering from infection or sepsis, can also be incorporated into any of a pharmaceutical composition containing a compound described herein. All of these pharmaceutical compositions also preferably other inert carriers or excipients as appropriate for the formulation desired, e.g., as discussed herein. Therefore, in certain embodiments the pharmaceutical compositions contain only one active agent each, and some pharmaceutical compositions are combinations of two or more active agents in one composition.

In some aspects, the invention described herein comprises a pharmaceutically acceptable compound described herein or pharmaceutically acceptable composition comprising a compound described herein, packaged together in a pharmaceutical pack or kit with a package insert (label) approved by a government agency responsible for regulating pharmaceutical agents, e.g., the FDA or EMA, wherein the label includes use of the compound) s) described herein, alone or in combination.

### H. Doses and Regimens

Treatment regimens suitable for the inventive methods include a single administration or a course of administrations lasting an hour, multiple hours, a full day, or longer, including administration for the remainder of the subject's life. The regimen can include one dose or multiple doses per day, for example, an immediate infusion administration lasting for an hour, multiple hours, a full day, or longer.

Dosage amounts per administration include any amount determined by the practitioner, and will depend on the size of the subject to be treated, the state of the health of the subject, the route of administration, the condition to be treated or prevented, and the like.

Dose ranges that are effective and well tolerated can be selected by one of ordinary skill in the art. Those of ordinary skill in the art will also understand that certain group of drugs can be used in combination with other group of drugs, and that an “effective amount” of such an agent for treating a disorder may be an amount such that the therapeutic effect of interest is produced by the combination of the drugs and the other therapies, also used at their effective amounts. Optionally, a dose may be tailored to the particular recipient, for example, through administration of increasing doses until a preselected desired response is achieved. If desired, the specific dose level for any particular subject may be selected based at least in part upon a variety of factors including the activity of the specific compound employed, the particular condition being treated and/or its severity, the age, body weight, general health, route of administration, and/or any concurrent medication.

In some embodiments, a 5-HT antagonist, partial antagonist, or inverse agonist is used in an amount sufficient to decrease one or more biological activities of 5-HT by at least 20%, 30%, 40%, 50%, 60%, 70%, 80%, 90%, 95%, or 100% relative to a suitable control. In some embodiments a 5-HT antagonist, partial antagonist, or inverse agonist decreases the biological activity of 5-HT by reducing binding of serotonin to 5-HT_{2A} by at least 20%, 30%, 40%, 50%, 60%, 70%, 80%, 90%, 95%, or 100% relative to a suitable control.

In some embodiments, an RTK inhibitor is used in an amount sufficient to reduce edema by at least 20%, 30%, 40%, 50%, 60%, 70%, 80%, 90%, 95%, or 100% relative to a suitable control, e.g., between 50% and 75%, 75% and 90%, or 90% and 100%.

In some embodiments, a dietary supplement for folic acid or iron is used in an amount sufficient to reduce edema by at least 20%, 30%, 40%, 50%, 60%, 70%, 80%, 90%, 95%, or 100% relative to a suitable control, e.g., between 50% and 75%, 75% and 90%, or 90% and 100%.

In some embodiments, an antihistamine drug is used in an amount sufficient to reduce edema by at least 20%, 30%, 40%, 50%, 60%, 70%, 80%, 90%, 95%, or 100% relative to a suitable control, e.g., between 50% and 75%, 75% and 90%, or 90% and 100%.

In some embodiments, fenspiride hydrochloride or levomepromazine is used in an amount sufficient to reduce edema by at least 20%, 30%, 40%, 50%, 60%, 70%, 80%, 90%, 95%, or 100% relative to a suitable control, e.g., between 50% and 75%, 75% and 90%, or 90% and 100%.

In some embodiments, an SSRI is used in an amount sufficient to reduce edema by at least 20%, 30%, 40%, 50%, 60%, 70%, 80%, 90%, 95%, or 100% relative to a suitable control, e.g., between 50% and 75%, 75% and 90%, or 90% and 100%.

In some embodiments, an MAO inhibitor is used in an amount sufficient to reduce edema by at least 20%, 30%, 40%, 50%, 60%, 70%, 80%, 90%, 95%, or 100% relative to a suitable control, e.g., between 50% and 75%, 75% and 90%, or 90% and 100%.

A suitable control in the context of assessing or quantifying the effect of a drug of interest is typically a comparable biological system (e.g., cells or a subject) that has not been exposed to or treated with the agent of interest, e.g., P. aeruginosa LPS 60 μg/mL (or zebrafish not treated with the drug of interest).

In certain embodiments, an effective dose of a composition as described herein can be administered to a patient once. In certain embodiments, an effective dose of a composition as described herein can be administered to a patient repeatedly. For systemic administration, subjects can be administered a therapeutic amount of 5-HT antagonist, partial agonist or inverse agonist, RTK inhibitor, and/or folic acid and/or iron supplements containing composition as described herein, such as, e.g. 0.01 mg/kg, 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more of total compound amount per weight of subject.

The dosage of 5-HT antagonist, partial agonist or inverse agonist, or RTK inhibitor, and/or folic acid and/or iron supplements as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from minutes to days depending on a number of clinical factors, such as the subject's sensitivity to a composition as described herein. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily. Examples of dosing and/or treatment schedules are administration daily, twice daily, three times daily or four or more times daily over a period of 1 day or more. A composition as described herein can be administered over a period of time, such as over a 5 minute, 10 minute, 15 minute, 20 minute, or 25 minute period.

### EXAMPLES

### Example 1. Materials and Methods

### 1.1. Zebrafish Husbandry

Adult Danio rerio (zebrafish AB strain) from in-house breeding stocks were used to produce high quality embryos used for testing. The zebrafish stocks are outcrossed periodically with new stocks of AB zebrafish obtained from the Zebrafish International Resource Center (Eugene, OR, USA). Zebrafish were maintained at an Association for Assessment and Accreditation of Laboratory Animal Care International (AAALAC) approved facility at the Walter Reed Army Institute for Research (WRAIR). All experiments were performed in compliance with AAALAC guidelines.

The zebrafish colony was maintained on a 14-h light and 10-h dark photoperiod under full spectrum LED lighting. The adult zebrafish colony was housed in either flow-through aquaculture racks or in tanks with these water quality conditions: water temperature 27.5±1.5° C.; dissolved oxygen 7.52±0.3 mg/L; pH 7.56±0.3; alkalinity 110 to 180 mg/L as CaCO₃; hardness 150 to 210 mg/L as CaCO₃; conductivity 651±75 μS/cm; and total ammonia less than 0.1 mg/L as NH₃. Adult zebrafish were fed three times daily on weekdays: 2 feedings of Gemma Micro 300 (Skretting Zebrafish, Westbrook, ME) and 1 feeding of live brine shrimp nauplii (Brine Shrimp Direct, Ogden, UT). On weekends, adult zebrafish received 2 feedings: one Gemma Micro 300 and one live brine shrimp nauplii.

Adult zebrafish aged 6-18 months were used to supply healthy embryos for chemical screening. The day prior to testing, ˜60 zebrafish were selected and used for breeding. Equal numbers of males and females were placed into two iSPAWN-S (Techniplast, West Chester, PA, USA) breeding chambers. A divider placed in each iSPAWN-S breeding chamber confined 15 females to the bottom of the chamber, and 15 male zebrafish were placed into the top section. Lids prevented the zebrafish from jumping out of the chambers. I-SPAWN chambers used a recirculating water system, with temperature maintained at 28.5°±1° C. At about 6:30 AM or 1 h after the lights came on the next morning, the lid and divider were removed to allow breeding. The spawning platform of the breeding chamber was raised up to its upper most position to confine the zebrafish to shallow water and stimulate breeding. This method produced >1000 embryos per breeding event per breeding chamber. After 15 min, the spawning platform was lowered and the embryos were collected by placing a fine-mesh strainer under the valve located at the bottom of the breeding chamber and opening the valve only part of the way so that the embryos slowly come out without being damaged. Once the embryos were collected they were transferred to glass Petri dishes (˜300-400 embryos/dish) containing fresh fish culture water and placed into an incubator shielded from light at 28.5°±1° C. At the conclusion of the study, all surviving larvae were euthanized with sodium hypochlorite according to the AVMA Guidelines for Euthanasia of Animals, 2020.

### 1.2. Zebrafish Model Optimization: LPS and Endpoint Screening

The endotoxicity/sepsis zebrafish model used was based on the method of Philip et al. (2017). In this model, larval zebrafish (3 days post fertilization (dpf)) were exposed to Escherichia coli 0111:B4 LPS by immersion in microplate wells for 24 hours, and three endpoints were measured: mortality, tail edema, and ROS production. To facilitate rapid and repeatable drug screening for rescue from LPS-induced endotoxicity, the responsiveness of these three endpoints to alternative LPS sources were determined.

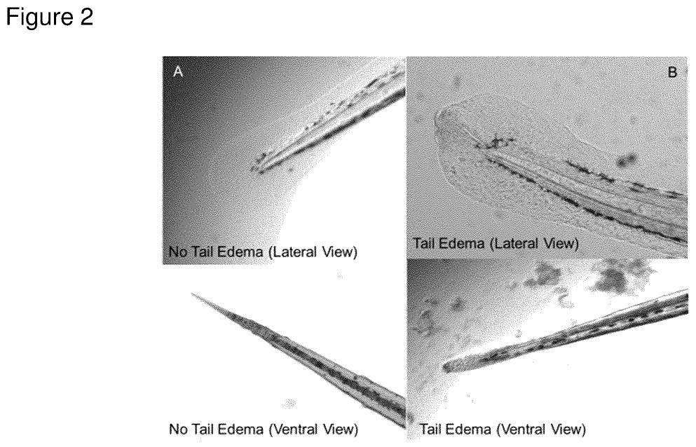

Three sources of LPS were evaluated: E. coli 0111:B4, P. aeruginosa Sigma L9143, and K. pneumoniae. E. coli 0111:B4 LPS was selected as the reference LPS used by Philip et al. (2017). P. aeruginosa LPS is commonly associated with infections in hospitals, accounting for an estimated 32,600 cases and resulting in an estimated 2,700 deaths in 2017 (CDC Report 2019). P. aeruginosa infections are also a common cause of sepsis in both patients with severe burns and immunocompromised patients. K. pneumoniae LPS has military relevance related to battlefield injury infections (Kiley et al., 2021). Initial range finding LC50 tests with larval zebrafish were conducted for each LPS strain at both 3-dpf and 5-dpf. For the 5-dpf LC50 range find test, 15 zebrafish embryos were exposed in 6 well microplates containing 3.5 mL of 0, 25, 50, 100, 150, and 200 μg/mL of LPS in EM. (E. coli 0111:B4 was also tested at higher concentrations, 400, 800, and 1000 μg/mL due a lack of toxicity within the initial dose range.) Well plates were then covered with parafilm and placed into an incubator at 28.5° C. for 24 hours. After 6 and 24 hours, the plates were removed and scored for mortality, tail edema, and ROS production. The tail edema endpoint evaluates vascular leakage from caudal fin in response to LPS exposure (FIG. 2). ROS production was determined as described by Philip et al. (2017) by adding 2,7-dichloroflurescein diacetate (100 μM) to the wells containing the zebrafish larvae and incubating them in a 100% dark photoperiod incubator at 28.5° C. for one hour. Florescent images of the incubated zebrafish were then captured with a Keyence BZ-X710 fluorescent microscope using 10× objective with a GFP filter and fixed 1/15 second exposure for all imaging. The 3-dpf LC50 range find was the same as the 5 dpf range find with the following changes: 96 well plates were used in place of the 6 well plates, the number of zebrafish was increased from 15 to 32, and the dose ranges tested for P. aeruginosa and K. pneumoniae were changed to 10, 20, 30, 40, 50, 60, 70, 80, 90, and 100 g/mL due to the steep mortality curve in the 5-dpf range find. Results from the LPS toxicity testing and endpoint screening were used to develop the optimized testing procedure through evaluation in selected model compounds, as described in the next section and in the results.

### 1.3. Model Compound Testing

The optimized zebrafish sepsis model was evaluated against compounds previously shown to have efficacy in previous zebrafish sepsis models: fasudil, hydrocortisone, dexamethasone, and fisetin. Fasudil, an anti-vascular leakage compound (inhibitor of the RhoA/Rho-kinase pathway) was shown by Philip et al. (2017) to rescue zebrafish from LPS-induced mortality, ROS production, and tail fin edema. Fasudil also rescued LPS-induced vascular leakage in murine models of experimental sepsis (Li et al., 2010) and reduced acute lung injury in septic rats through inhibition of the Rho/ROCK signaling pathway (Wang et al., 2018). Hsu et al. (2018) found that the corticosteroids hydrocortisone and dexamethasone produced responses similar to a protein tyrosine phosphatase inhibitor (Shp2 (Ptpn11a) inhibitor 11a-1), leading to decreased inflammatory cytokines, increased il-10 anti-inflammatory activity, reduced tissue damage and preservation of vascular junction proteins in their zebrafish model. Fisetin is a dietary flavonoid that showed inhibition of LPS-induced inflammation and reduced mortality from endotoxic shock in zebrafish larvae; possible mechanisms include crosstalk between GSK-3β/β-catenin and the NF-κB signaling pathways (Molagoda et al., 2021). Endpoints evaluated with model compounds included mortality, tail edema, and ROS at 6- and 24-hours post exposure to varying dose ranges of LPS.

### 1.4. Custom Drug Library Selection

Four commercially available drug discovery libraries including nearly 2500 compounds were selected to evaluate drug targets known to affect pathways implicated in the initiation and progression of sepsis in humans (Adadey et al. (2018), Bergmann et al. (2021), Dare et al. (2009), Huerta and Rice (2019)), including inflammation, mitochondrial dysfunction, coagulation, and apoptosis. Libraries selected included the Selleckchem Cytokine Inhibitor Library (L9500), the Selleckchem Highly Selective Inhibitor Library (L3500), the MedChemExpress Mitochondria-Targeted Compound Library (HY-L089), and the MedChemExpress Coagulation and Anti-coagulation Compound Library (HY-L136). To reduce the high costs associated with developing new or novel drugs, this initial set of candidate compounds was cross-referenced with Selleckchem FDA-Approved Drug Library (L1300) and MedChemExpress FDA-Approved Drug Library (HY-L022). This evaluation identified 644 drugs for sepsis efficacy screening in the optimized zebrafish endotoxicity/sepsis model.

### 1.5. Initial Drug Screening

Healthy, normally developing embryos collected from the adult AB zebrafish spawning events were treated with 75 μM of 1-phenyl 2-thiourea (PTU) in EM prior to 24 hours post fertilization (hpf) to block pigmentation formation (Karlsson et al, 2001). These embryos were then placed into an incubator at 28.5° C. until they reached 3-dpf. Embryos were then removed from the incubator and plated individually into the wells of 96 well plates containing 50 μL of EM.

A super stock of 120 μg/mL of P. aeruginosa LPS was made up in EM in a glass container. This stock was then used to make sub stocks of 120 μg/mL P. aeruginosa LPS (positive control) and 120 μg/mL P. aeruginosa LPS containing 20 μM of test drug in glass scintillation vials. Immediately after the sub stocks were made, the wells were dosed with either 50 μL of 120 μg/mL of P. aeruginosa, 120 μg/mL of P. aeruginosa with 20 μM of test drug, or EM (1:1 Dilution). Each 96 well plate had one LPS positive control (n=16), 4 different test drugs with P. aeruginosa LPS (n=16 per drug), and one negative control (n=16). Once the plates were dosed, they were covered with parafilm and placed into an incubator at 28.5° C. The plates were removed at 6- and 24-hours post exposure to be scored for tail edema and mortality. All 644 drugs evaluated for rescue from mortality and tail edema induced by P. aeruginosa LPS exposure as compared to LPS-treated controls after 24 hours of exposure.

### 1.6. Top Rescue Drug Confirmatory Testing and Down Selection

To further down select the top rescue drugs, only drugs that had >60% rescue of mortality and tail edema induced by P. aeruginosa LPS exposure from the initial screen were further evaluated in confirmatory testing. Confirmatory testing followed the same methods as the initial testing but used 5-dpf larvae instead of the 3-dpf larvae that were used for the initial screening because they were easier to handle and replicated the methods of Philip et al. (2017). The 5-dpf larvae have more fully developed organ functionality than 3-dpf larvae, and in a meta-analysis of acute toxicity data for 600 chemicals, Ducharme et al. (2015) found that larval zebrafish acute toxicity data were predictive of rodent and rabbit acute toxicity for 4 and 5-dpf larvae, but not for 3-dpf larvae.

In a second stage of confirmatory testing, drugs that had >80% rescue of mortality and tail edema with the 5-dpf larvae were evaluated further. First, a concentration range find test was used to determine the potential therapeutic window for the drugs. Briefly, 5-dpf zebrafish embryos were placed into the wells of the 96 well plate (one embryo per well), with each well containing 50 μL of EM. Then, an additional 50 μL of either 120 μg/mL of P. aeruginosa or 120 μg/mL of P. aeruginosa with the test drug (1:1 dilution) was added to the 96 well plate. Each 96 well plate had one positive control (n=16) and 4 different concentrations of the test drug with P. aeruginosa LPS (n=16 per concentration). Once the plates were dosed, they were covered with parafilm and placed into an incubator at 28.5° C. The plates were removed at 6- and 24-hours post exposure to be scored for tail edema and mortality. The drug concentrations tested were 10, 1, 0.1, 0.01 and 0.001 μM.

Drugs identified with >80% rescue were subjected to additional testing. Drug pretreatment for 24 hours prior to LPS challenge was evaluated to determine if any prophylactic benefit could be realized with any of the top rescue drugs. The intent was to determine if drug pre-treatment might be used at the point of injury as a preventative therapeutic before the onset of infection and development of sepsis. Parallel groups of fish received a 24-hour drug pre-treatment or no drug pre-treatment prior to LPS challenge. For this test, 4 dpf PTU-treated zebrafish embryos were placed into 96 well plates containing 100 μL of EM. The 24-hour drug pre-treatment group had a 10 μM dose of test drug added when the zebrafish embryos were 4 dpf to allow for a 24-hour pre-treatment prior to LPS exposure at 5-dpf. A parallel group that did not receive 24-hour drug pre-treatment was evaluated to provide comparative results due to the slight change in LPS challenge/toxicity required to complete the studies. All plates were covered with parafilm and placed into an incubator at 28.5° C. for 24 hours. After 24 hours, all treatments had an additional 100 μL of either 120 μg/mL of P. aeruginosa, 120 μg/mL of P. aeruginosa with test drug (to maintain a 10 μM final in well drug concentration and 60 μg/mL of P. aeruginosa LPS), or EM alone (control group). Once the plates were dosed, they were covered with parafilm and placed into an incubator at 28.5° C. The plates were removed at 6- and 24-hours post exposure and scored for tail edema and mortality. Once the 24-hour tail edema and mortality endpoints were collected, a neurobehavioral test was conducted as described in section 2.8.

The top 3 drugs (Ketanserin, Tegaserod, and Brexpiprazole) identified from the confirmatory testing were also tested against another strain of LPS to determine if the candidate drug showed efficacy in more than one LPS strain. These tests followed the same methods described in the 5-dpf zebrafish confirmatory testing, but K. Pneumoniae LPS (45 μg/mL final in well concentration) was used instead of P. aeruginosa LPS.

### 1.7. Neurobehavioral Testing

All groups were evaluated at the end of the study using a neurobehavioral assay to evaluate deviations from normal fish behavior and further evaluate rescue from sepsis, as well as to determine if any of the drugs might be exhibiting off-target side effects. This assay is a modified version of the larval zebrafish light dark transition assay with an added UV light (wavelength=295 nm) phase to capture any compound specific neurological effects to UV light. (Basnet et al., 2019, Kokel et al., 2013, Peng et al., 2016). The light period assesses deviations from normal locomotor behavior while the rapid transition to dark provides a measure of stress response by identifying any deviations from the normal heightened locomotor activity caused by the transition to dark. The neurobehavioral assay used a custom trial control script created by Noldus Ethovision XT 15, and it was executed in a Noldus DanioVision system. Zebrafish larvae in microplates were allowed to habituate in the DanioVision system for 5 minutes under white light conditions prior to the start of the test. The neurobehavioral assay consisted of three phases: a 3-minute light phase (white light), a 3-minute dark phase (no light) and a 5 second UV phase (UV light only). The Danio Vision system captured larval zebrafish movement throughout this assay at 30 frames per second.

### 1.8. Statistical Analysis

LC50s for the LPS tests were determined using the Trimmed Spearmen-Karber method (Hamilton et al., 1977). The tail edema and mortality data from the initial and confirmatory drug testing was analyzed using GraphPad Prism 9 using Fisher's Exact Test. Each drug treatment group was compared pairwise with the on-board positive control group on each test plate. Statistical significance was determined with values of p≤0.05. Neurological behavioral testing data was analyzed by Ethovision software that produced an activity analysis profile per frame for each well of the 96 well plates. Any dead embryos were removed from the data set, and the remaining data set was imported into Excel. The offset function in Excel was used to average the percentage of pixel change per 0.1 s for each plate. These averages were then used to calculate the area under the curve (AUC) for each concentration and each phase of the neurological behavioral tests. The behavioral data was then statistically analyzed by drug per phase using a 2 tailed t-test to determine statistical significance p≤0.01.

### Example 2. Model Optimization

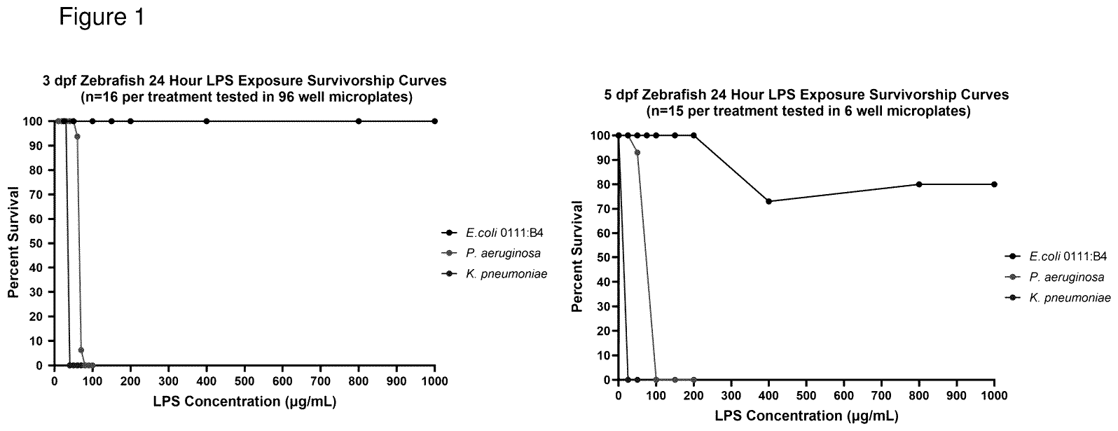

Of the three bacterial strains of LPS tested, K. Pneumoniae LPS was the most toxic, with a 24-hour exposure 5-dpf zebrafish LC50 of <25 μg/mL and a 24-hour exposure 3-dpf zebrafish LC50 of 34.6 μg/mL (95% confidence limits could not be calculated). The second most toxic LPS tested was P. aeruginosa with a 24-hour exposure to 5-dpf zebrafish LC50 of 67.5 μg/mL (95% confidence limits 61.8-73.8 μg/mL) and a 24-hour exposure to 3-dpf zebrafish LC50 of 64.71 μg/mL (95% confidence limits of 63.0-66.5 μg/mL). E. coli LPS was over an order of magnitude less toxic to zebrafish with an LC50 of >1000 μg/mL for both 24-hour exposures to the 5-dpf and the 3-dpf zebrafish embryos. (FIG. 1).

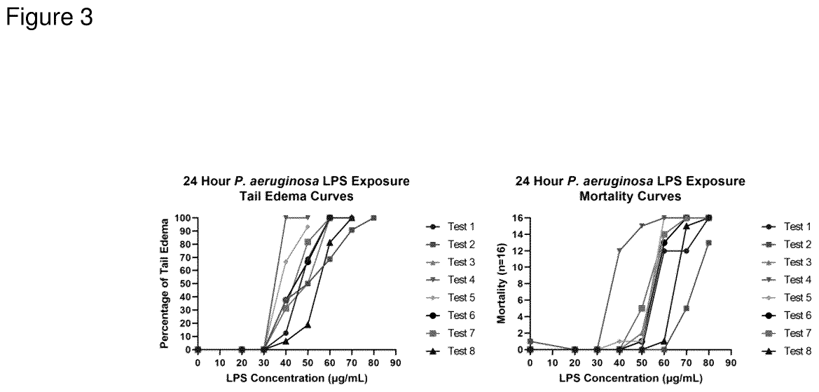

P. aeruginosa LPS endpoint screening optimization was conducted to select a single LPS concentration that would produce a consistent endotoxic effect to facilitate reliable and repeatable drug compound screening studies. Specifically, the goal was to obtain near 50% mortality with 24-hour LPS exposed fish while generating a 100% combined response of tail edema and mortality. FIG. 2 shows an example of a mild tail edema indictive of LPS endotoxicity in larval zebrafish. FIG. 3 shows LPS dose response curves for tail edema and mortality during the protocol optimization and reference compound studies. Based on these response data, a concentration of 60 μg/mL P. aeruginosa LPS was found to be optimal for eliciting mortality and tail edema responses suitable for demonstrating the rescue potential of candidate drugs.

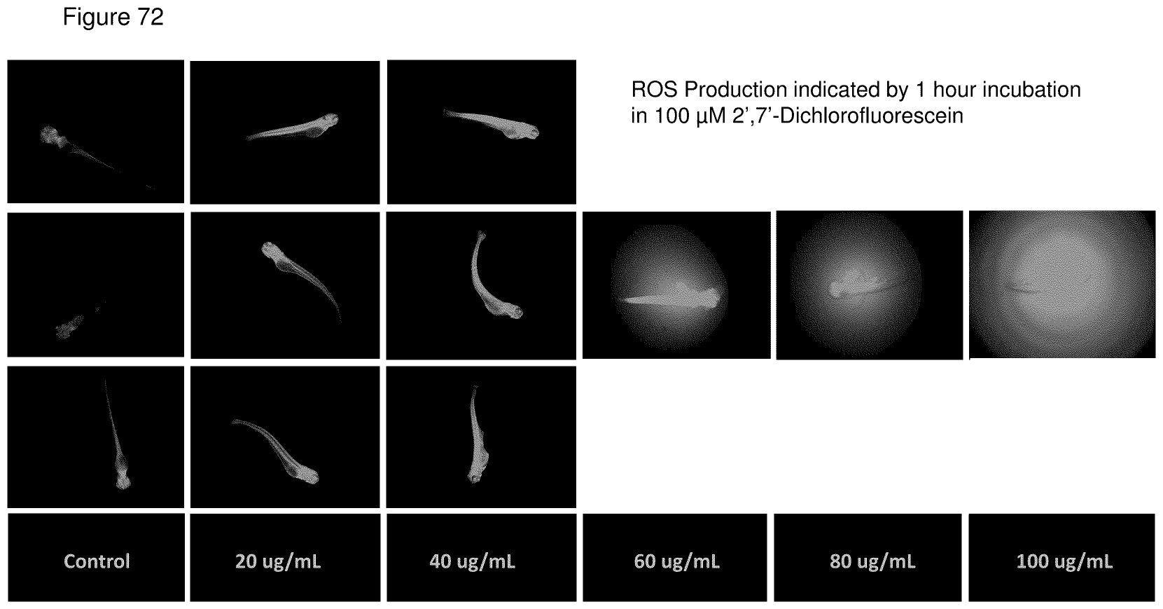

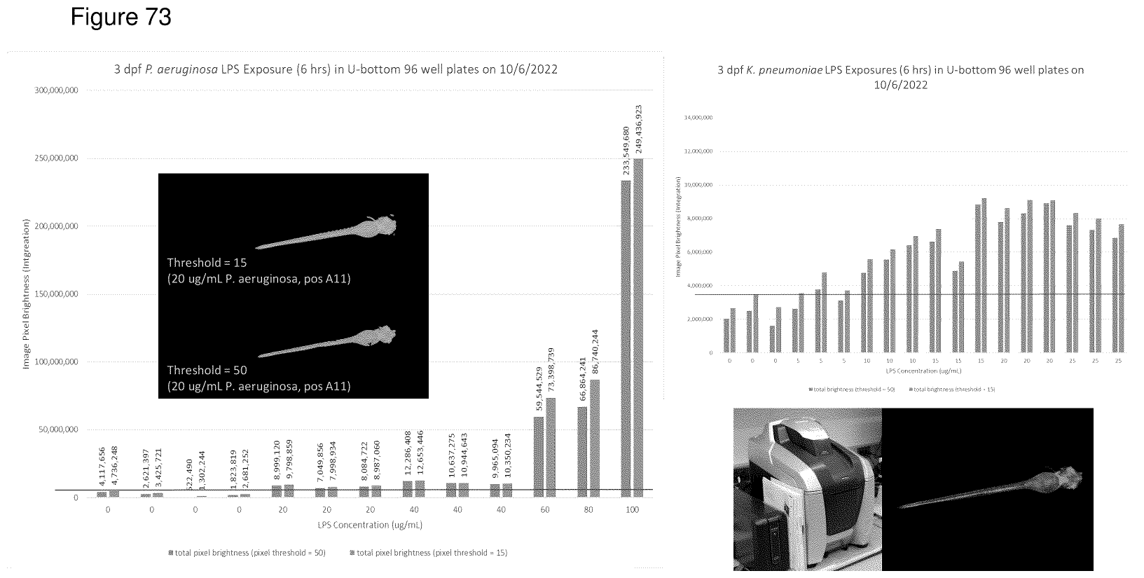

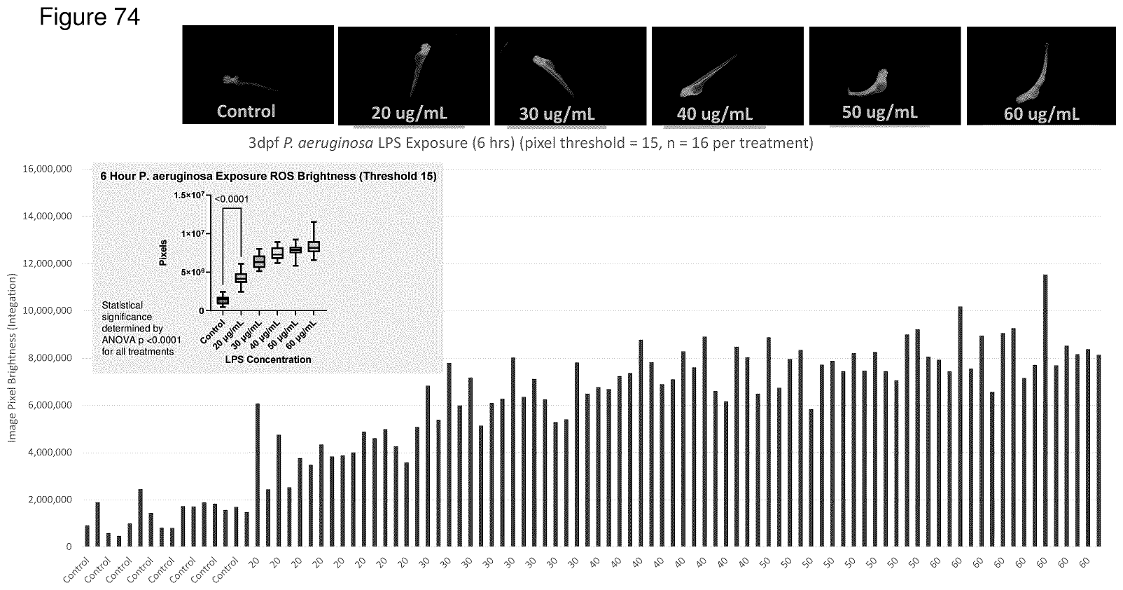

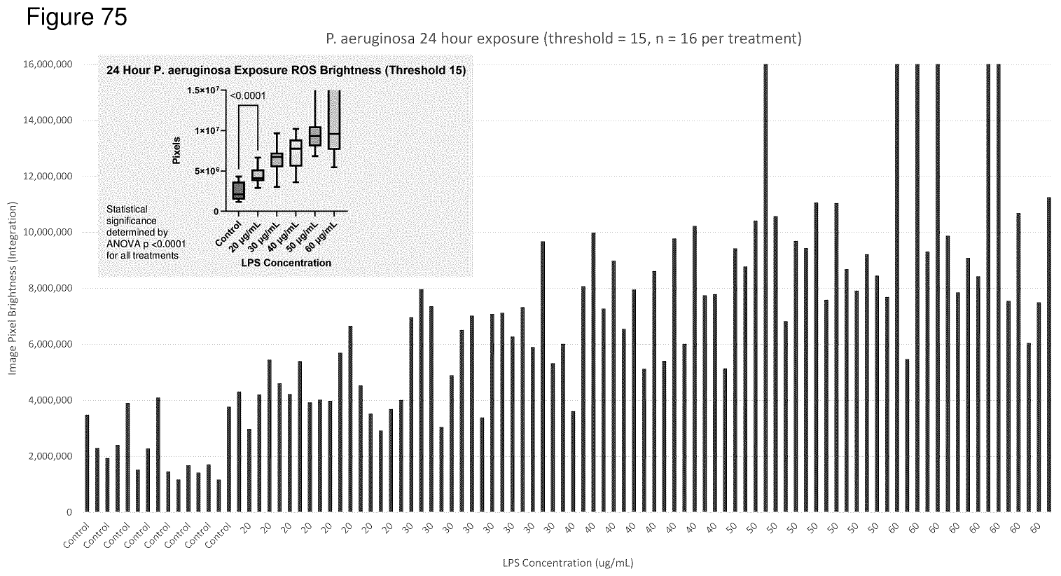

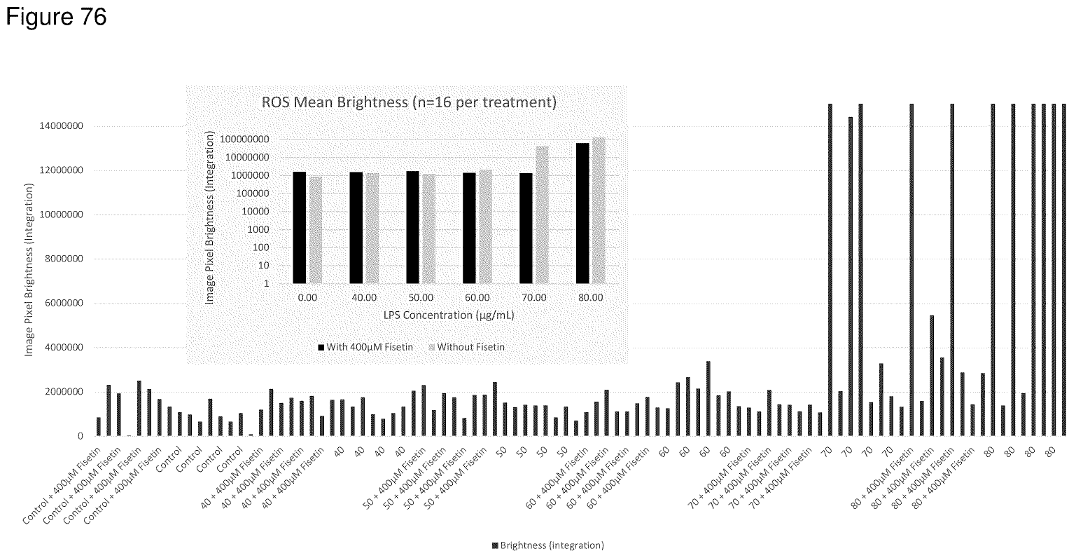

While the ROS endpoint did show a quantifiable and statistically significant dose response to increasing concentrations of LPS (see FIGS. 72-76), it was not included as a drug screening endpoint. The additional time requirement for ROS measurements could not be justified given that mortality and tail edema together provided reliable and reproducible data sufficient for targeting efficacious drugs to prevent or treat sepsis.

### Example 3. Model Compound Testing

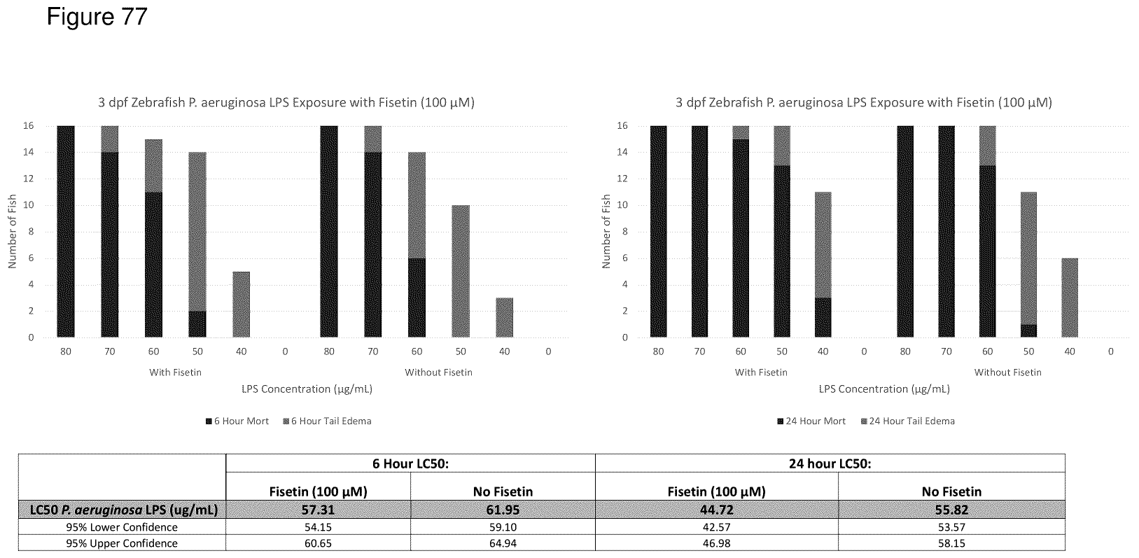

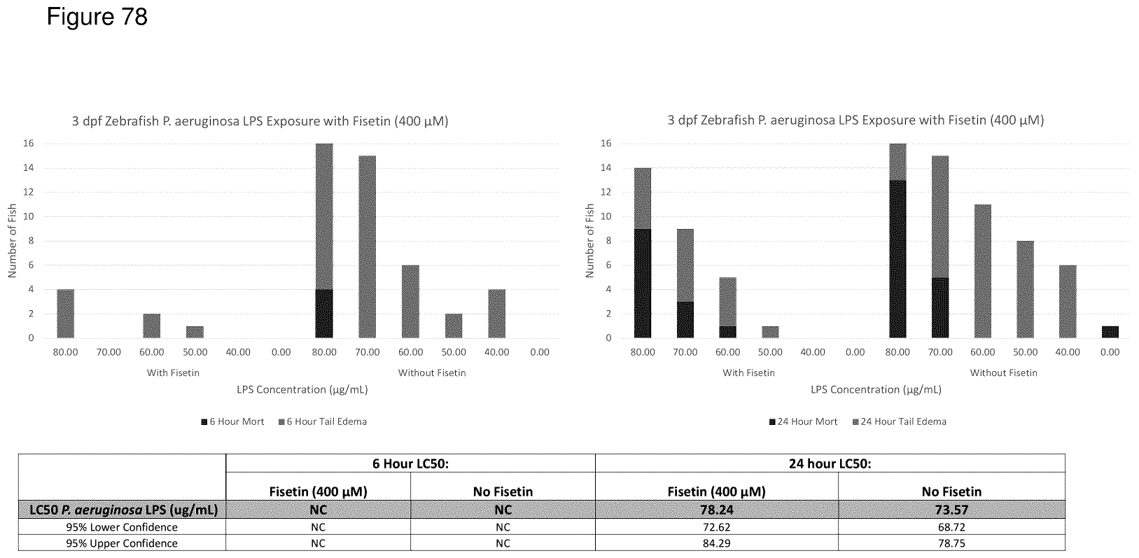

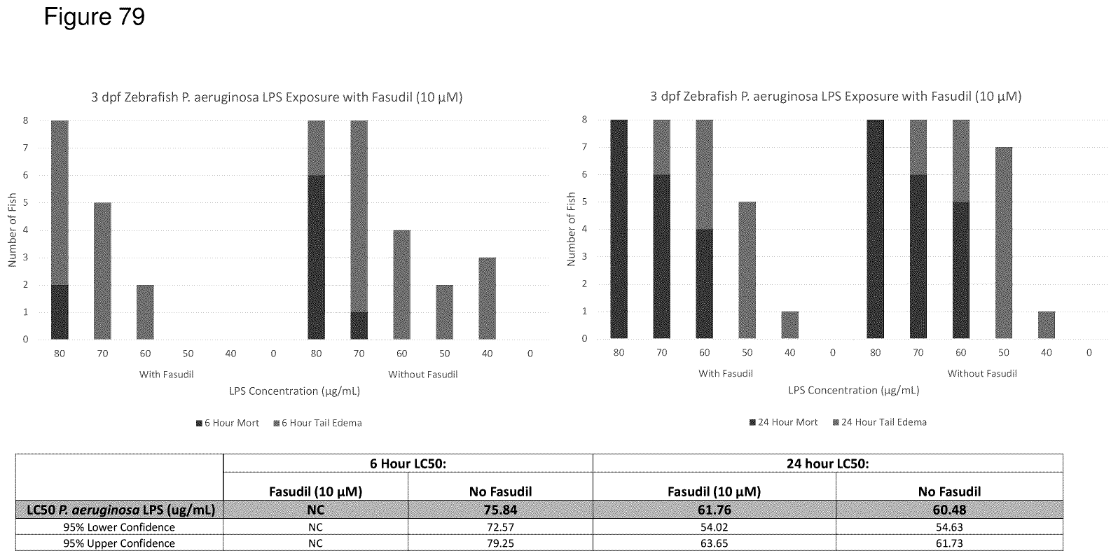

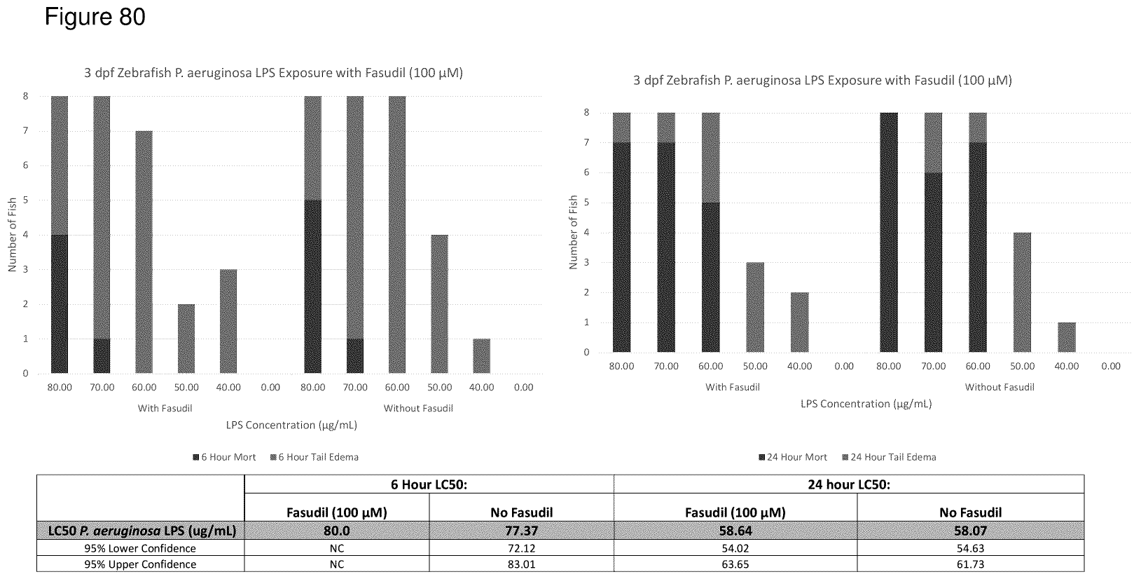

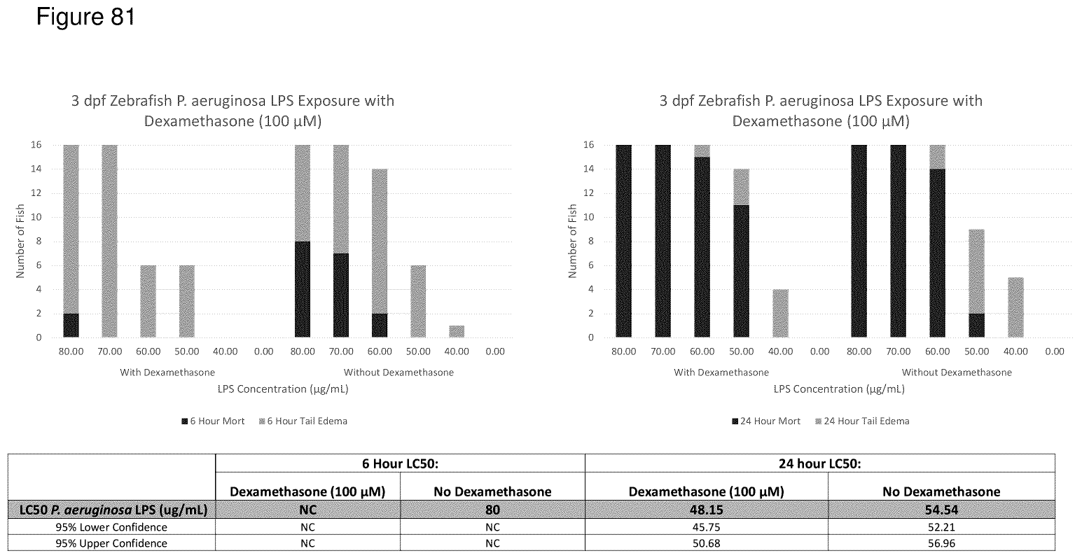

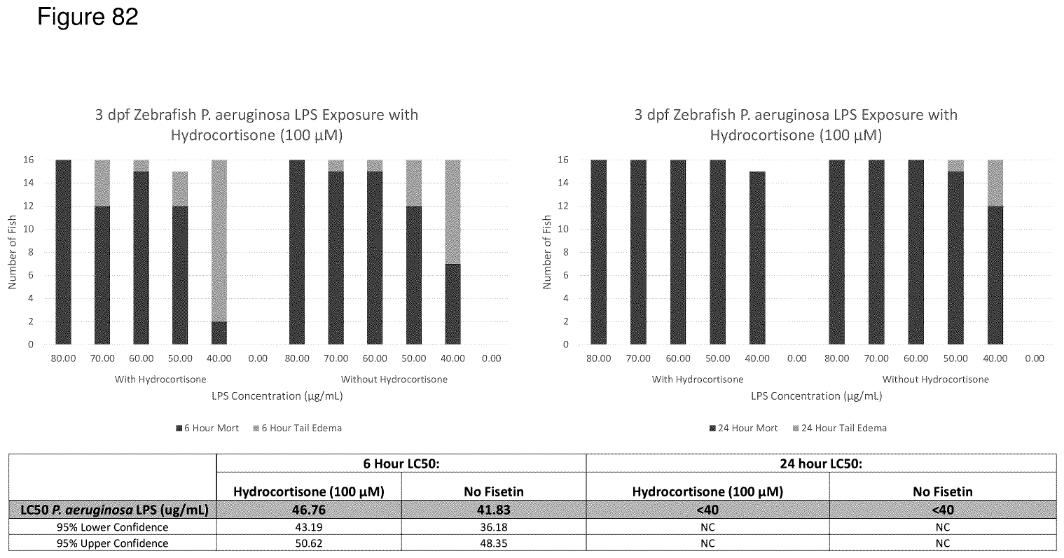

Four model compounds (fasudil, hydrocortisone, dexamethasone, and fisetin) identified from prior published zebrafish LPS studies were evaluated. LPS LC50 values increased to 78.2 (95% confidence limits 72.6-84.3 μg/mL) from 73.6 μg/mL (95% confidence limits 68.7-78.8 μg/mL) with and without Fisetin (400 μM) respectively and to 61.8 (95% confidence limits 57.4-66.5 μg/mL) from 60.5 μg/mL (95% confidence limits 56.3-65.0 μg/mL) with and without Fasudil (10 μM). Neither compound showed complete rescue in any of the exposed fish at the 60 μg/mL LPS challenge concentration, although Fasudil did show a statistically significant (p=0.0233) reduction in mortality at 24 hours post exposure. Prior work with Fisetin by Molagoda et al. (2021) and other work by Hsu et al. (2018) used LPS microinjection methods and mortality rates in LPS positive controls for those studies were 30% (after 36 hours) and 5% (after 24 hours) respectively. The LPS challenge used in this study led to >60% mortality in LPS positive controls and thus direct comparisons are not advised given the differing exposure route and increased endotoxic challenge. Likewise, for Fasudil, our selection of LPS (P. aeruginosa rather than E. coli) differed from prior work and may have been responsible for some of the differences in efficacy when compared to our study. For dexamethasone, the preliminary 6-hour reading did show some level of efficacy, with a reduction in mortality across concentrations, although 24-hour readings resulted in greater mortality with dexamethasone than with no treatment; the LPS LC50 values were 48.2 μg/mL with dexamethasone (100 μM) and 54.5 μg/mL without dexamethasone. Hydrocortisone showed a similar response, with some efficacy noted at 6 hours post LPS exposure but with increased mortality over no treatment at 24 hours post exposure. FIGS. 77-82 provide data related to these findings.

### Example 4. Initial Drug Screening

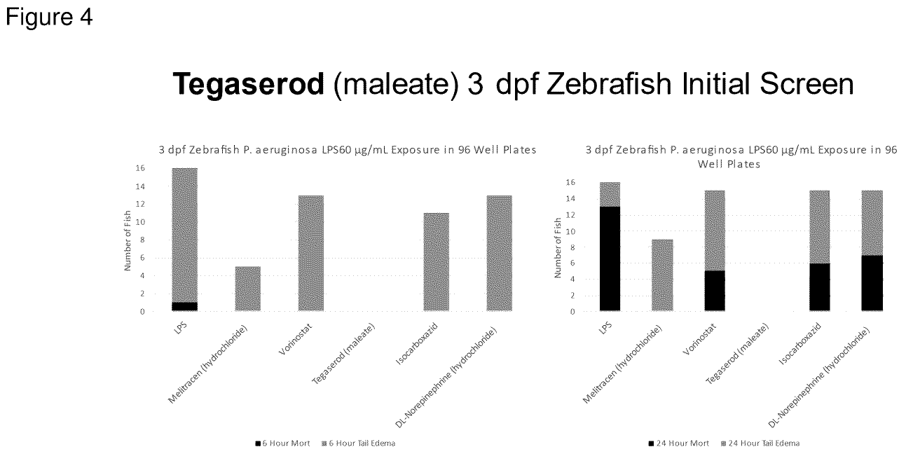

Screening of the sepsis-focused custom FDA-approved drug library of 644 compounds yielded 29 compounds that exceeded the threshold of 60% rescue of tail edema and mortality; 4 compounds provided 100% rescue (ketanserin, tegaserod, brexpiprazole, and dapoxetine). FIG. 4 shows the response of tegaserod along with other drugs from the initial screening library that were tested on the same 96 well plate. For this test plate, tegaserod was the only drug that showed efficacy at preventing significant tail edema and mortality.

Of the 29 compounds that exceeded 60% rescue, 19 came from the coagulation/anti-coagulation pathway library, 8 came from the mitochondria-targeted pathway library, and 2 each were from the cytokine targeted pathways and the highly selective targeted pathways. Two of the 29 top compounds were classified in more than one library. Sunitinib was in the mitochondria targeted library but also in the cytokine targeted library. Iproniazid was in the mitochondria target library as well as the highly selective library. Hit rates within a library were highest for the coagulation/anti-coagulation and the mitochondria-targeted pathway libraries as shown in table 15.

TABLE 15
Summary of Novel Rapid Drug Discovery Model for Sepsis

| | | Number of | |
|---|---|---|---|
| | Number of | Compounds | |
| | Compounds | Exceeding | Hit Rate |
| Compound Library | Evaluated | 60% Rescue | (>60% Rescue) |
| MedChemExpress | 295 | 19 | 6.4% |
| Coagulation and | | | |
| Anti-Coagulation | | | |
| Targeted Pathways | | | |
| (FDA approved only) | | | |
| MedChem Express | 136 | 8 | 5.8% |
| Mitochondria- | | | |
| Targeted Pathways | | | |
| (FDA approved only) | | | |
| Selleckchem Cytokine | 140 | 2 | 1.4% |
| Targeted Pathways | | | |
| (FDA approved only) | | | |
| Selleckchem | 114 | 2 | 1.7% |
| Highly Selective | | | |
| Targeted Pathways | | | |
| (FDA approved only) | | | |

Note:
Only 644 compounds were tested (not 685) as duplicates between pathway libraries were not tested.

### Example 5. Top Rescue Drug Confirmatory Testing and Down Selection

Confirmatory testing of the top 29 compounds showing >60% rescue in the initial drug screening effort yielded 16 compounds that demonstrated >80% rescue using 5-dpf larvae. Five of the 16 compounds showed 100% rescue of tail edema and mortality at 5-dpf (ketanserin tartrate, tegaserod, brexpiprazole, sunitinib and paliperidone palmitate).

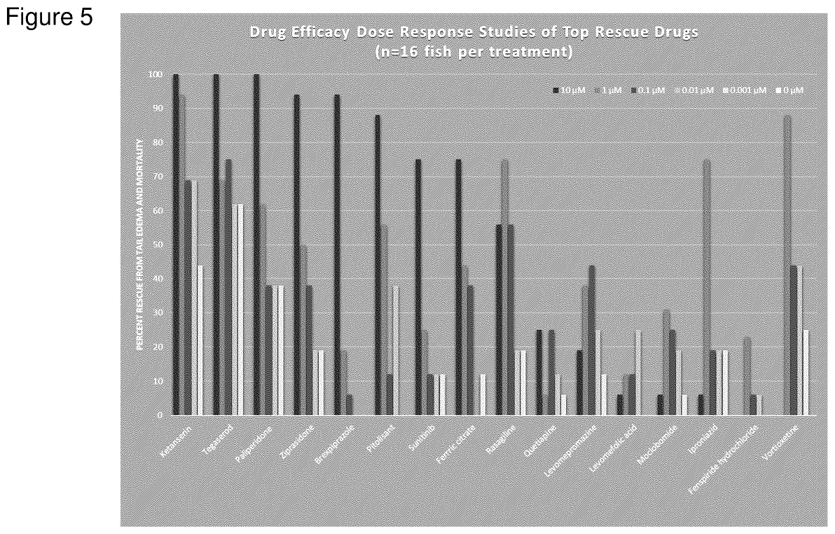

Full drug efficacy dose range response curves for the top 16 compounds are shown in FIG. 5. Ketanserin and tegaserod showed >60% rescue at drug concentrations as low as 0.01 μM, providing the widest therapeutic window when compared to other top rescue compounds, none of which had similar rescue below 1 μM.

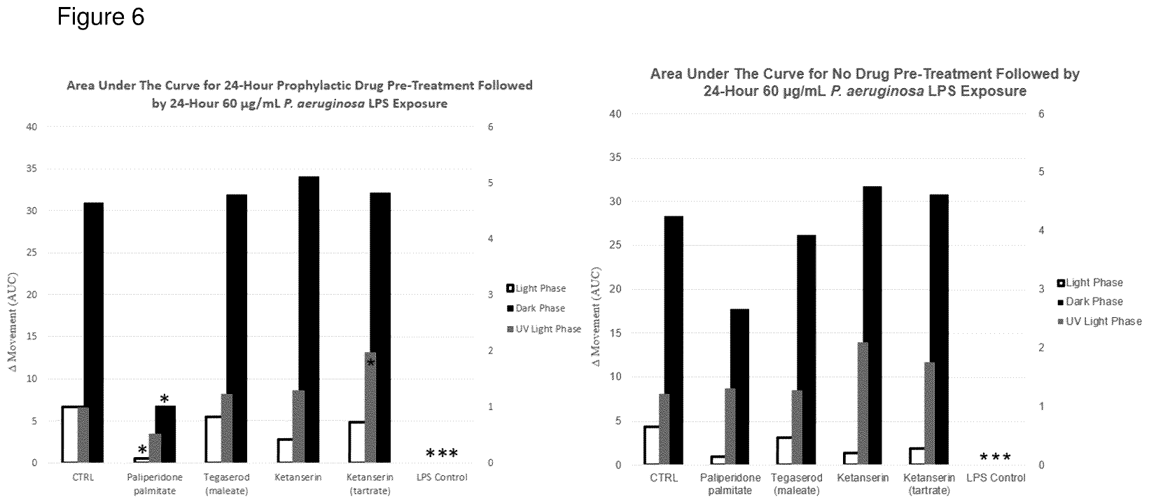

Further evaluation of the top 16 rescue compounds used a 24-hour drug pre-treatment prior to 24-hour LPS exposure. Because of the increased LPS exposure in the pre-treatment dosing studies, there was increased LPS toxicity, leading to 100% mortality in the LPS positive controls. Drug pre-treatment generally didn't improve rescue for most of the top drugs other than sunitinib and ziprasidone (Table 16). The light/dark neurobehavioral testing (FIGS. 6 & 7) identified 3 drugs (ketanserin, tegaserod, and brexpiprazole) that showed little to no difference from negative controls. No significant differences were noted in any of these three drugs other than brexpiprazole, which showed hyperactivity (p=0.005) during the dark phase with LPS exposure without 24-hour drug pre-treatment but was not significant when given prophylactically 24 hours prior to LPS exposure. Ketanserin, tegaserod, and brexpiprazole were further challenged with a lethal concentration of K. Pneumoniae LPS (45 μg/mL). Both brexpiprazole and tegaserod showed 100% rescue of tail edema and mortality, while ketanserin showed 88% rescue. Thus, these drugs show strong efficacy against endotoxicity not only from P. aeruginosa LPS, but also from K. Pneumoniae LPS.

TABLE 16
Summary of 24-hour pretreatment test with the compounds

| | 24 hour Tail Edema | | 24 Hour Mortality | | 24 Hour Mortality and Tail Edema | |
|---|---|---|---|---|---|---|
| | n = 16 fish treatment | | n = 16 fish treatment | | n = 16 fish treatment | |
| | No-Drug | Drug | No Drug | Drug | No-Drug | Drug |
| Compound Name | Pretreatment | Pretreatment | Pretreatment | Pretreatment | Pretreatment | Pretreatment |
| Ketanserin | 0 | 0 | 0 | 0 | 0 | 0 |
| (tartrate) | | | | | | |
| Ketanserin | 0 | 0 | 0 | 0 | 0 | 0 |
| Brexpiprazole | 1 | 0 | 0 | 1 | 1 | 1 |
| Tegaserod | 1 | 0 | 1 | 0 | 2 | 0 |
| (maleate) | | | | | | |
| Sunitnib (malate) | 4 | 1 | 1 | 1 | 5 | 2 |
| Sunitinib | 4 | 1 | 3 | 3 | 7 | 4 |
| Ziprasidone | 2 | 0 | 5 | 4 | 7 | 4 |
| (Hydrochloride | | | | | | |
| monohydrate) | | | | | | |
| Paliperidone | 3 | 2 | 6 | 10 | 9 | 12 |
| palmitate | | | | | | |
| Ziprasidone | 2 | 4 | 8 | 6 | 10 | 10 |
| pitolisant | 2 | 0 | 13 | 16 | 15 | 16 |
| Ferric citrate | 2 | 0 | 13 | 15 | 15 | 15 |
| Fenspiride | 0 | 0 | 16 | 16 | 16 | 16 |
| (hydrochloride) | | | | | | |
| Levomefolic acid | 1 | 0 | 15 | 16 | 16 | 16 |
| levomepromazine | 1 | 0 | 15 | 16 | 16 | 16 |
| Rasagiline | 0 | 0 | 16 | 16 | 16 | 16 |
| (mesylate) | | | | | | |
| vortioxetine | 0 | 0 | 16 | 16 | 16 | 16 |
| Quetiapine | 1 | 1 | 15 | 15 | 16 | 16 |
| (hemifumarate) | | | | | | |
| Moclobemide | 0 | 0 | 16 | 16 | 16 | 16 |
| Iproniazid | 1 | 0 | 15 | 16 | 16 | 16 |
| Bedaquiline | 0 | 0 | 16 | 16 | 16 | 16 |

TABLE 17
Drugs selected after screening using the zebrafish sepsis model

| | No Pre- | Pre- | Post | | |
|---|---|---|---|---|---|
| | Treatment | Treatment | Test | Known | Current Drug |
| | Rescue | Rescue | Behavioral | Receptor | Use Owners |
| | (n = 16) | (n = 16) | Assay | Targets | (Brand Name) |
| Ketanserin | 100% | 100% | Normal | 5-HT2a (inverse agonist) | Jansen |
| | | | | | (Sufrexal) |
| Tegaserod | 88% | 100% | Normal | 5-HT4 (partial agonist) | Alfasigma |
| | | | | 5-HT2a, b and c (antagonist) | (Zellnorm ™) |
| Brexpiprazole | 94% | 94% | Normal | 5-HT1a (partial agonist) | Otsuka |
| | | | | 5-HT2a (antagonist) | America |
| | | | | Noradenergic a1b (antagonist) | (Rexulti^{R}) |
| | | | | Noradenergic a2c (antagonist) | |
| | | | | Dopamine D2 (partial agonist) | |
| Sunitinib | 69% | 88% | Impaired | Inhibits multiple receptor | Pfizer |
| | | | neurobehavior | tyrosine kinases (RTKs) | (Sutent) |
| | | | from | Platelet-derived growth factor | |
| | | | incomplete | receptor alpha and beta, | |
| | | | rescue of | Vascular endothelial growth | |
| | | | endotoxicity | factor receptor 1, 2 and 3, | |
| | | | | Mast/stem cell growth factor | |
| | | | | Kit, RTKe FLT3, Macrophage | |
| | | | | colony stimulating factor 1, | |
| | | | | Hepatocyte growth factor | |
| | | | | receptor | |
| Ziprasidone | 56% | 75% | Impaired | Dopamine D2 (antagonist) | Pfizer |
| | | | neurobehavior | 5-HT2a, c and d (antagonist) | (Zeldox^{R}) |
| | | | from | 5-HT1a (agonist) | |
| | | | incomplete | | |
| | | | rescue of | | |
| | | | endotoxicity | | |

### Example 6. Initial Sepsis Compound Library Screening Summary (644 FDA Approved Compounds)

3-dpf embryos were challenged with 60 μg/mL P. aeruginosa LPS in combination with single 10 μM concentration of each compound for 24 hours (n=16 fish per compound). Among them, 4 compounds showed 100% rescue of tail edema and mortality, which are ketanserin, tegaserod, brexpiprazole, and dapoxetine. Other 29 compounds exceeded 60% rescue of tail edema and mortality at 24 hours post exposure.

Next, follow-on 5-dpf screening was implemented to confirm initial screening with more larvae. Zebrafish swim faster at 5-dpf (requiring more time to load 96 plates), and all major organ systems are functioning at 5-dpf and thus determined as advantageous for follow-on confirmation. LPS toxicity appears to be similar with 3-dpf embryos and 5-dpf larvae. Five compounds (ketanserin, tegaserod, brexpiprazole, sunitinib and paliperidone palmitate) showed 100% rescue of tail edema and mortality at 5-dpf. However, dapoxetine which showed 100% rescue at 3-dpf only showed 38% rescue at 5-dpf.

Ciprofloxacin, a quinolone antibiotics, was also tested at 5-dpf larvae to confirm low rescue seen at 3-dpf embryos (as standard of care antibiotic).

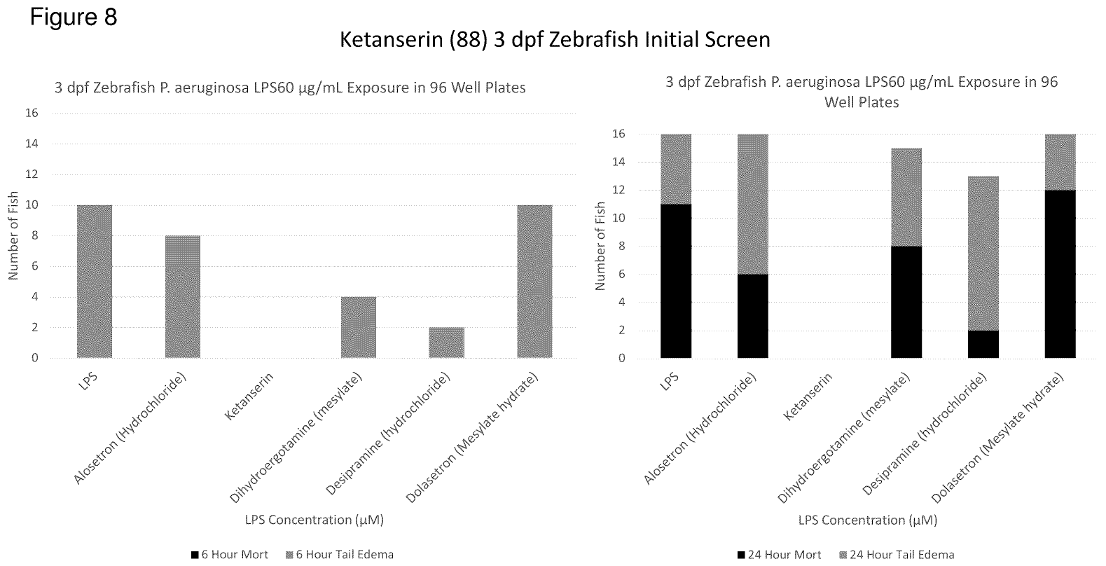

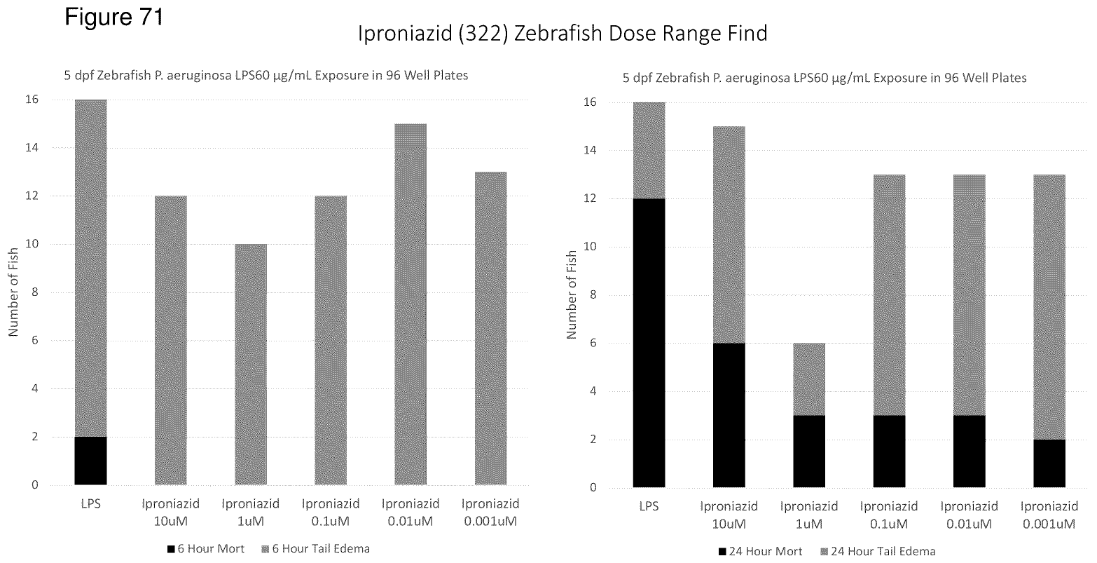

Ziprasidone (hydrochloride monohydrate) was also evaluated at 5-dpf even though it only showed 44% rescue at 3-dpf (to compare to Ziprasidone which showed 63% rescue at 3-dpf). Both compounds had 100% rescue at 5-dpf. Overall, 16 compounds exceeded 80% rescue at 5-dpf as seen on slide 5. Therapeutic dose range finding was performed (10, 1, 0.1, 0.01, 0.001 μM) for these 16 compounds. FIGS. 8-71 provide results of initial screening.

### Example 7. Leading Sepsis Rescue Compounds (10 μM) after 3-Dpf and 5-Dpf Evaluations

Below is the summary of 644 FDA approved compounds as presumptive sepsis therapeutics at a concentration 10 UM of the compounds after 3-dpf and 5-dpf zebrafish.
(1) The sepsis rescue rate of ketanserin tartrate (Compound 437) was 100% at 3-dpf and 5-dpf, and ketanserin (Compound 88) 100% and 81% rescue 3-dpf and 5-dpf, respectively.
(2) The sepsis rescue rate of tegaserod maleate (Compound 400) was 100% at 3-dpf and 5-dpf.
(3) Although the sepsis rescue rate of paliperidone (Compound 61) was only 6% at 3-dpf (not tested at 5-dpf) and that of risperidone (Compound 4), the parent compound of paliperidone, was only 6%, the sepsis rescue rates of paliperidone palmitate (Compound 429) were 94% and 100% at 3-dpf and 5-dpf, respectively. (Differences in schizophrenia relapse in humans with injectable paliperidone palmitate vs oral extended release paliperidone https://www.ncbi.nlm.nih.gov/pmc/articles/PMC3722005/).
(4) The sepsis rescue rates of quetiapine hemifumerate (Compound 11) were 94% and 81% at 3-dpf and 5-dpf, respectively.
(5) The sepsis rescue rates of ziprasidone (Compound 361) were 63% and 100% at 3-dpf and 5-dpf, respectively, and those of ziprasidone hydrochloride monohydrate (Compound 63) were 44% and 100% at 3-dpf and 5-dpf, respectively.
(6) The sepsis rescue rates of brexpiprazole (Compound 45) were 100% and 94% at 3-dpf and 5-dpf, respectively.
(7) The sepsis rescue rates of sunitinib malate (Compound 621) were 88% and 100% at 3-dpf and 5-dpf, respectively, and those of sunitinib malate (Compound 354) and sunitinib (Compound 97) were 88 and 81% at 3-dpf and 100% rescue at 5-dpf. All 3 sutinib compounds had yellow crystal precipitate during all tests.
(8) The sepsis rescue rates of pitolisant (Compound 435) were 94% and 88% at 3-dpf and 5-dpf, respectively. However, the sepsis rescue rates of pitolisant hydrochloride (Compound 589 and 126) were only 56% and 38% at 3-dpf (not tested at 5-dpf, but dose range finding was completed for comparison to compound 435).
(9) The sepsis rescue rates of fenspiride hydrochloride (Compound 17) were 63% and 88% at 3-dpf and 5-dpf, respectively.
(10) The sepsis rescue rates of levomepromazine (Compound 316) were 63% and 88% at 3-dpf and 5-dpf, respectively
(11) The sepsis rescue rates of levomefolic acid (Compound 344) were 63% and 88% at 3-dpf and 5-dpf, respectively.
(12) The sepsis rescue rates of ferric citrate (Compound 340) were 94% and 81% at 3-dpf and 5-dpf, respectively.
(13) The sepsis rescue rates of vortioxetine (Compound 376) were 63% and 88% at 3-dpf and 5-dpf, respectively
(14) The sepsis rescue rates of rasagiline mesylate (Compound 314) were 63% and 88% at 3-dpf and 5-dpf, respectively.
(15) The sepsis rescue rates of moclobemide (Compound 5) were 75% and 81% at 3-dpf and 5-dpf, respectively.
(16) The sepsis rescue rates of iproniazid (Compound 322) were 63% and 81% at 3-dpf and 5-dpf, respectively.

TABLE 18
Summary of Sepsis Compound Library Screen using 3-dpf Zebrafish Embryos Challenged with LPS (P. aeruginosa 60 μg/mL)

| Compound | Product | Percent Rescue | | |
|---|---|---|---|---|
| Number | Name | (Mort + Tail Edema) | Target | Pathway |
| 45 | Brexpiprazole | 100% | 5-HT Receptor; Adrenergic | GPCR/G Protein; Neuronal |
| 156 | Dapoxetine (hydrochloride) | 100% | Serotonin Transporter | Neuronal Signaling |
| 88 | Ketanserin | 100% | 5-HT Receptor; Autophagy; | Autophagy; GPCR/G Protein; |
| 437 | Ketanserin (tartrate) | 100% | 5-HT Receptor; Autophagy; | Autophagy; GPCR/G Protein; |
| 400 | Tegaserod (maleate) | 100% | 5-HT Receptor; Apoptosis | Apoptosis; GPCR/G Protein; |
| 474 | Bedaquiline fumarate | 94% | Anti-infection | Microbiology |
| 340 | Ferric citrate | 94% | Antibiotic; Reactive Oxygen | Anti-infection; |
| 429 | Paliperidone palmitate | 94% | 5-HT Receptor; Dopamine | GPCR/G Protein; Neuronal |
| 435 | Pitolisant | 94% | Histamine Receptor | GPCR/G Protein; |
| 11 | Quetiapine (hemifumarate) | 94% | 5-HT Receptor; Dopamine | GPCR/G Protein; Neuronal |
| 621 | Sunitinib Malate | 94% | c-Kit, PDGFR,VEGFR | Protein Tyrosine Kinase |
| 8 | Gabexate (mesylate) | 88% | Factor Xa; Proteasome | Metabolic Enzyme/Protease |
| 37 | Sertraline (hydrochloride) | 88% | Serotonin Transporter | Neuronal Signaling |
| 354 | Sunitinib (Malate) | 88% | Apoptosis; Autophagy; IRE1; | Apoptosis; Autophagy; Cell |
| 7 | Tranylcypromine (hemisulfate) | 88% | Histone Demethylase; | Epigenetics; Neuronal Signaling |
| 97 | Sunitinib | 81% | Apoptosis; Autophagy; IRE1; | Apoptosis; Autophagy; Cell |
| 31 | Avatrombopag | 75% | Thrombopoietin Receptor | Immunology/Inflammation |
| 227 | Bedaquiline | 75% | Antibiotic; Bacterial | Anti-infection |
| 378 | Ceritinib dihydrochloride | 75% | Anaplastic lymphoma kinase | Protein Tyrosine Kinase/RTK |
| 30 | Lumateperone (tosylate) | 75% | 5-HT Receptor; Dopamine | GPCR/G Protein; Neuronal |
| 5 | Moclobemide | 75% | Monoamine Oxidase | Neuronal Signaling |
| 44 | Terfenadine | 75% | Apoptosis; Caspase; Histamine | Apoptosis; GPCR/G Protein; |
| 138 | Alverine (citrate) | 69% | 5-HT Receptor | GPCR/G Protein; Neuronal |
| 155 | Dapsone | 69% | Antibiotic; Bacterial; Parasite; | Anti-infection; |
| 6 | Almotriptan (malate) | 63% | 5-HT Receptor | GPCR/G Protein; Neuronal |
| 17 | Fenspiride (hydrochloride) | 63% | Histamine Receptor; | GPCR/G Protein; |
| 322 | Iproniazid | 63% | Monoamine Oxidase | Neuronal Signaling |
| 344 | Levomefolicacid | 63% | DNA/RNA Synthesis; | Cell Cycle/DNA Damage; |
| 316 | Levomepromazine | 63% | 5-HT Receptor; Dopamine | GPCR/G Protein; Neuronal |
| 10 | Levonordefrin | 63% | Adrenergic Receptor | GPCR/G Protein; Neuronal |
| 314 | Rasagiline (mesylate) | 63% | Apoptosis; Autophagy; | Apoptosis; Autophagy; Neuronal |
| 376 | Vortioxetine | 63% | 5-HT Receptor; Serotonin | GPCR/G Protein; Neuronal |
| 361 | Ziprasidone | 63% | 5-HT Receptor; Dopamine | GPCR/G Protein; Neuronal |
| 3 | Avatrombopag (hydrochloride) | 56% | Thrombopoietin Receptor | Immunology/Inflammation |
| 310 | Betahistine | 56% | Histamine Receptor | GPCR/G Protein; |
| 549 | Lidocaine hydrochloride | 56% | EGFR | Angiogenesis |
| 318 | Mebhydrolin | 56% | Histamine Receptor | GPCR/G Protein; |
| 12 | Pindolol | 56% | 5-HT Receptor; Adrenergic | GPCR/G Protein; Neuronal |
| 589 | Pitolisant hydrochloride | 56% | Histamine Receptor | Neuronal Signaling |
| 598 | Quizartinib (AC220) | 56% | FLT3 | Angiogenesis |
| 157 | Chlorphenoxamine | 50% | Histamine Receptor | GPCR/G Protein; |
| 386 | Duloxetine (hydrochloride) | 50% | Serotonin Transporter | Neuronal Signaling |
| 516 | Gefitinib (ZD1839) | 50% | EGFR | Protein Tyrosine Kinase |
| 207 | Hydroxyzine (pamoate) | 50% | Histamine Receptor | GPCR/G Protein; |
| 317 | Imeglimin (hydrochloride) | 50% | Mitochondrial Metabolism; | Immunology/Inflammation; |
| 438 | Naratriptan (hydrochloride) | 50% | 5-HT Receptor | GPCR/G Protein; Neuronal |

29 compounds exceeding a 60% rescue threshold were selected for follow-on evaluations using 5-dpf embryos at 10 μM concentration to confirm initial 3-dpf response.
Note: Initial screen was performed with 3-dpf embryos based upon literature methods of Phillip et al.

TABLE 19
Leading Rescue Compounds Evaluated with 5-dpf Larvae Combined Effects (Tail
Edema and Mortality) (Initial 3-dpf Screen Results Shown for Reference)

| | | 3 dpf | 5 dpf | | |
|---|---|---|---|---|---|
| | | Embryos | Embryos | | |
| | | Percent | Percent | | |
| | | Rescue | Rescue | | |
| | | (Mort + | (Mort + | | |
| Compound | Compound | Tail | Tail | | |
| Number | Name | Edema) | Edema) | Target | Pathway |
| 437 | Ketanserin (tartrate) | 100% | 100% | 5-HT Receptor; | Autophagy; GPCR/G |
| | | | | Autophagy, Potassium | Protein; Membrane |
| 400 | Tegaserod (maleate) | 100% | 100% | 5-HT Receptor; | Apoptosis; GPCR/G |
| | | | | Apoptosis | Protein; Neuronal |
| 429 | Paliperidone palmitate | 94% | 100% | 5-HT Receptor, | GPCR/G Protein; |
| | | | | Dopamine Receptor | Neuronal Signaling |
| 621 | Sunitinib Malate | 94% | 100% | c-Kit, PDGFR, VEGFR | Protein Tyrosine Kinase |
| 354 | Sunitinib (Malate) | 88% | 100% | Apoptosis; Autophagy; | Apoptosis; Autophagy; |
| | | | | IRE1; Mitophagy; | Cell Cycle/DNA |
| 97 | Sunitinib | 81% | 100% | Apoptosis; Autophagy; | Apoptosis; Autophagy; |
| | | | | IRE1; Mitophagy; | Cell Cycle/DNA |
| 361 | Ziprasidone | 63% | 100% | 5-HT Receptor; | GPCR/G Protein; |
| | | | | Dopamine Receptor | Neuronal Signaling |
| 63 | Ziprasidone (hydrochloride | 44% | 100% | 5-HT Receptor; | GPCR/G Protein; |
| | monohydrate) | | | Dopamine Receptor | Neuronal Signaling |
| 45 | Brexpiprazole | 100% | 94% | 5-HY Receptor; | GPCR/G Protein; |
| | | | | Adrenergic Receptor; | Neuronal Signaling |
| 435 | Pitolisant | 94% | 88% | Histamine Receptor | GFCR/G Protein; immunology/ |
| | | | | | inflammation; |
| 17 | Fenspiride (hydrochloride) | 63% | 88% | Histamine Receptor; | GPCR/G Protein; immunology/ |
| | | | | Phosphodiesterase | inflammation; |
| 344 | Levomefolic acid | 63% | 88% | DNA/RNA Synthesis; | Cell Cycle/DNA Damage; |
| | | | | Endogenous | |
| 316 | Levomepromazine | 63% | 88% | 5-HT Receptor; | GPCR/G Protein; |
| | | | | Dopamine Receptor | Neuronal Signaling |
| 314 | Rasagiline (mesylate) | 63% | 88% | Apoptosis; Autophagy; | Apoptosis; Autophagy; |
| | | | | Monoamine | Neuronal Signaling |
| 375 | Vortioxetine | 63% | 88% | 5-HT Receptor, | GPCR/G Protein; |
| | | | | Serotonin Transporter | Neuronal Signaling |
| 88 | Ketanserin | 100% | 81% | 5-HT Receptor; | Autophagy; GPCR/G |
| | | | | Autophagy; Potassium: | Protein; Membrane |
| 340 | Ferric citrate | 94% | 81% | Antibiotic; Reactive | Anti-infection; immunology/ |
| | | | | Oxygen Species | inflammation; |
| 11 | Quetiapine (hemifumarate) | 94% | 81% | 5-HT Receptor; | GPCR/G Protein; |
| | | | | Dopamine Receptor | Neuronal Signaling |
| 5 | Moclobemide | 75% | 81% | Monoamine Oxidase | Neuronal Signaling |
| 322 | Iproniazid | 63% | 81% | Monoamine Oxidase | Neuronal Signaling |
| 227 | Bedaquiline | 75% | 75% | Antibiotic; Bacterial | Anti-infection |
| 10 | Levonordefrin | 63% | 75% | Adrenergic Receptor | GPCR/G Protein; |
| | | | | | Neuronal Signaling |
| 6 | Aimotriptan (malate) | 63% | 63% | 5-HT Receptor | GPCR/G Protein; |
| | | | | | Neuronal Signaling |
| 7 | Tranylcypromine (hemisulfate) | 88% | 56% | Histone Demethylase; | Epigenetics; Neuronal |
| | | | | Monoamine | Signaling |
| 156 | Dapoxetine (hydrochloride) | 100% | 38% | Serotonin Transporter | Neuronal Signaling |
| 378 | Ceritinib dihydrochloride | 75% | 38% | Anaplastic lymphoma | Protein Tyrosine |
| | | | | kinase (ALK); IGF-1R; | Kinase/RTK |
| 138 | Alverine (citrate) | 69% | 38% | 5-HT Receptor | GPCR/G Protein; |
| | | | | | Neuronal Signaling |
| 8 | Gabexate (mesylate) | 88% | 31% | Factor Xa; Proteasome | Metabolic Enzyme/Protease |
| 37 | Sertraline (hydrochloride) | 88% | 31% | Serotonin Transporter | Neuronal Signaling |
| 31 | Avatrombopag | 75% | 31% | Thrombopoietin Receptor | Immunology/inflammation |
| 155 | Dapsone | 69% | 25% | Antibiotic; Bacterial; | Anti-infection; immunology/ |
| | | | | Parasite; Reactive | inflammation; |
| 30 | Lumateperone (tosylate) | 75% | 19% | 5-HT Receptor; | GPCR/G Protein; |
| | | | | Dopamine Receptor | Neuronal Signaling |
| 34 | Ciprofloxacin (hydrochloride | 13% | 6% | Antibiotic; Apoptosis: | Anti-infection; Apoptosis; |
| | monohydrate) | | | Bacterial; | Cell Cycle/DNA |
| 474 | Bedaquiline fumarate | 94% | 0% | Anti-infection | Microbiology |
| 44 | Terfenadine | 75% | 0% | Apoptosis; Caspase; | Apoptosis: GPCR/G Protein; |
| | | | | Histamine Receptor; | |

### REFERENCES

1. Aboyoussef, A. M., Mohammad, M. K., Abo-Saif, A. A., & Messiha, B. A. (2021). Granisetron attenuates liver injury and inflammation in a rat model of cecal ligation and puncture-induced sepsis. Journal of Pharmacological Sciences, 147 (4), 358-366. https://doi.org/10.1016/j.jphs.2021.08.005
2. Adadey, S. M., Yakass, M. B., Agyemang, S., & Duodu, S. (2019). The Modulatory Effect of Lead Drug Candidates on Inflammatory Gene Expression in Sepsis: A Mini-Review. Current drug discovery technologies, 16 (1), 48-56. https://doi.org/10.2174/1570163815666180227162926
3. Ahern, G. P. (2011). 5-HT and the immune system. Current opinion in pharmacology, 11 (1), 29-33. https://doi.org/10.1016/j.coph.2011.02.004
4. Allen, J. P., & Neely, M. N. (2010). Trolling for the ideal model host: zebrafish take the bait. Future microbiology, 5 (4), 563-569. https://doi.org/10.2217/FMB.10.24
5. AVMA Guidelines for Euthanasia of Animals, 2020. American Veterinary Medical Association. ISBN 978-1-882691-09-8, Version 2020.0. Guidelines-on-Euthanasia-2020.pdf (avma.org)
6. Barber, A. E., Fleming, B. A., & Mulvey, M. A. (2016). Similarly lethal strains of extraintestinal pathogenic Escherichia coli trigger markedly diverse host responses in a zebrafish model of sepsis. mSphere, 1 (2), e00062-00016. https://doi.org/10.1128/mSphere.00062-16
7. Basnet, R. M., Zizioli, D., Taweedet, S., Finazzi, D., & Memo, M. (2019). Zebrafish Larvae as a Behavioral Model in Neuropharmacology. Biomedicines, 7 (1), 23.
8. Bergmann, C. B., Beckmann, N., Salyer, C. E., Hanschen, M., Crisologo, P. A., & Caldwell, C. C. (2021). Potential targets to mitigate trauma- or sepsis-induced immune suppression. Frontiers in Immunology, 12, 622601. https://doi.org/10.3389/fimmu.2021.622601
9. Bowman, T. V., & Zon, L. I. (2010). Swimming into the future of drug discovery: in vivo chemical screens in zebrafish. ACS chemical biology, 5 (2), 159-161. https://pubs.acs.org/doi/10.1021/cb100029t
10. Carius, B. M.; G. E. Bebarta; M. D. April; A. D. Fisher; J. Rizzo; P. Ketter; J. C. Wenke; V. S. Bebarta; S. G. Schauer. 2021. A retrospective analysis of combat injury patterns and prehospital interventions associated with the development of sepsis. Prehospital emergency care. https://doi.org/0.1080/10903127.2021.2001612
11. CDC. Antibiotic Resistance Threats in the United States, 2019. Atlanta, GA: U.S. Department of Health and Human Services, CDC; 2019. http://www.cdc.gov/drugresistance/Biggest-Threats.html
12. Chipiti, T; A. M. Viljoen; M. L. Cordero-Maldonado; C. G. Veale; F. R. Van Heerden. 2021. Anti-seizure activity of African medicinal plants: the identification of bioactive alkaloids from the stem bark of Rauvolfia caffra using an in vivo zebrafish model. Journal of ethnopharmacology 279:114282.
13. Clatworthy, A. E., Lee, J. S.-W., Leibman, M., Kostun, Z., Davidson, A. J., & Hung, D. T. (2009). Pseudomonas aeruginosa infection of zebrafish involves both host and pathogen determinants. Infection and immunity, 77 (4), 1293-1303.
14. Dare, A. J., Phillips, A. R., Hickey, A. J., Mittal, A., Loveday, B., Thompson, N., & Windsor, J. A. (2009). A systematic review of experimental treatments for mitochondrial dysfunction in sepsis and multiple organ dysfunction syndrome. Free Radical Biology and Medicine, 47 (11), 1517-1525. https://doi.org/10.1016/j.freeradbiomed.2009.08.019
15. Ducharme, N. A., Reif, D. M., Gustafsson, J.-A., & Bondesson, M. (2015). Comparison of toxicity values across zebrafish early life stages and mammalian studies: implications for chemical testing. Reproductive Toxicology, 55, 3-10.
16. Fleming, A., & Alderton, W. (2013). Zebrafish in pharmaceutical industry research: finding the best fit. Drug Discovery Today: Disease Models, 10 (1), e43-e50.
17. Forn-Cuní, G., Varela, M., Pereiro, P., Novoa, B., & Figueras, A. (2017). Conserved gene regulation during acute inflammation between zebrafish and mammals. Scientific reports, 7, Article 41905.
18. Gomes, M. C., & Mostowy, S. (2020). The case for modeling human infection in zebrafish. Trends in microbiology, 28 (1), 10-18. https://doi.org/10.1016/j.tim.2019.08.005
19. Guan, J., Liao, Y., Guo, Y., Yu, S., Wei, R., Niu, M., Gan, J., Zhang, L., Li, T., & Lv, J. (2022). Adjunctive granisetron therapy in patients with sepsis or septic shock (GRANTISS): A single-center, single-blinded, randomized, controlled clinical trial. Frontiers in Pharmacology, 13, 1013284. https://doi.org/10.3389/fphar.2022.1013284
20. Hamilton, M. A., Russo, R. C., & Thurston, R. V. (1977). Trimmed Spearman-Karber Method for Estimating Median Lethal Concentrations in Toxicity Bioassays. Environmental Science and Technology, 11 (7), 714-719-719. https://doi-org.wrair.idm.oclc.org/10.1021/es60130a004
21. Howe, K., Clark, M. D., Torroja, C. F., Torrance, J., Berthelot, C., Muffato, M., Collins, J. E., Humphray, S., McLaren, K., & Matthews, L. (2013). The zebrafish reference genome sequence and its relationship to the human genome. Nature, 496 (7446), 498-503.
22. Hsu, A. Y., Gurol, T., Sobreira, T. J., Zhang, S., Moore, N., Cai, C., Zhang, Z.-Y., & Deng, Q. (2018). Development and characterization of an endotoxemia model in zebra fish. Frontiers in Immunology, 9, 607.
23. Huang, Y., Ji, Q., Zhu, Y., Fu, S., Chen, S., Chu, L., Ren, Y., Wang, Y., Lei, X., & Gu, J. (2022). Activated platelets autocrine 5-hydroxytryptophan aggravates sepsis-induced acute lung injury by promoting neutrophils extracellular traps formation. Frontiers in cell and developmental biology, 9, 777989. https://doi.org/10.3389/fcell.2021.777989.
24. Huerta, L. E., & Rice, T. W. (2019). Pathologic difference between sepsis and bloodstream infections. The journal of applied laboratory medicine, 3 (4), 654-663. https://doi.org/10.1373/jalm.2018.026245
25. Johnston, A. M.; D. Easby; I. Ewington. 2013. Sepsis management in the deployed field hospital. BMJ Military Health. 159 (3): 175-180.
26. Karlsson, J., von Hofsten, J. & Olsson, PE. Generating Transparent Zebrafish: A Refined Method to Improve Detection of Gene Expression During Embryonic Development. Mar. Biotechnol. 3, 522-527 (2001). https://doi-org.wrair.idm.oclc.org/10.1007/s1012601-0053-4 Kiley J L, Mende K, Beckius M L, Kaiser S J, Carson M L, Lu D, et al. (2021) Resistance patterns and clinical outcomes of Klebsiella pneumoniae and invasive Klebsiella variicola in trauma patients. PLOS ONE 16 (8): e0255636. https://doi.org/10.1371/journal.pone.0255636
27. Keij, F. M.; B. E. V. Koch; F. L. Vigario; S. H. P. Simons; J. G. C. van Hasselt; et al. 2021. Zebrafish larvae as experimental model to expedite the search for new biomarkers and treatments for neonatal sepsis. Journal of Clinical and Translational Science 5 (1): E140.
28. Kelly, J. F.; A. E. Ritenour; D. F. Mclaughlin; K. A. Bagg; A. N. Apodaca; et al. 2008. Injury severity and causes of death from Operation Iraqi Freedom and Operation Enduring Freedom: 2003-2004 versus 2006. J Trauma Acute Care Surgery 64 (2): 521-527.
29. Kokel, D., Dunn, T. W., Ahrens, M. B., Alshut, R., Cheung, C. Y. J., Saint-Amant, L., Bruni, G., Mateus, R., van Ham, T. J., & Shiraki, T. (2013). Identification of nonvisual photomotor response cells in the vertebrate hindbrain. Journal of Neuroscience, 33 (9), 3834-3843.
30. Leber, A. T. (2019). Development of a bacterial infection model in zebrafish embryos with special focus on colistin-resistant Klebsiella pneumoniae Uppsala Universitet]. Uppsala, Sweden.
31. Li, Y., Wu, Y., Wang, Z., Zhang, X.-h., & Wu, W.-k. (2010). Fasudil attenuates lipopolysaccharide-induced acute lung injury in mice through the Rho/Rho kinase pathway. Medical Science Monitor, 16 (4), BR112-BR118. Li, Y.; Y. Wu; Z. Wang; X. Zhang; W. Wu. 2010. Fasudil attenuates lipopolysaccharide-induced acute lung injury in mice through the Rho/Rho kinase pathway. Med Sci Monit 16 (4): BR112-118.
32. Lieschke, G. J., & Currie, P. D. (2007). Animal models of human disease: zebrafish swim into view. Nature Reviews Genetics, 8 (5), 353-367.
33. Loes, A. N.; M. N. Hinman, D. R. Farnsworth, A. C. Miller, K. Guillemin; et al. 2021. Identification and characterization of zebrafish TLR4 coreceptor Md-2. Journal of Immunology 206 (5): 1046-1057.
34. Marcoleta, A. E., Varas, M. A., Ortiz-Severín, J., Vásquez, L., Berríos-Pastén, C., Sabag, A. V., Chávez, F. P., Allende, M. L., Santiviago, C. A., & Monasterio, O. (2018). Evaluating different virulence traits of Klebsiella pneumoniae using Dictyostelium discoideum and zebrafish larvae as host models. Frontiers in cellular and infection microbiology, 8, 30.
35. Masud, S., Torraca, V., & Meijer, A. H. (2017). Modeling infectious diseases in the context of a developing immune system. Current Topics in Developmental Biology, 124, 277-329. https://doi.org/10.1016/bs.ctdb.2016.10.006
36. Molagoda, I. M. N., Jayasingha, J. A. C. C., Choi, Y. H., Jayasooriya, R. G. P. T., Kang, C.-H., & Kim, G.-Y. (2021). Fisetin inhibits lipopolysaccharide-induced inflammatory response by activating β-catenin, leading to a decrease in endotoxic shock. Scientific reports, 11, 8377. https://doi.org/10.1038/s41598-021-87257-0
37. Neumann, J., Hofmann, B., Dhein, S., & Gergs, U. (2023). Cardiac roles of serotonin (5-HT) and 5-HT-receptors in health and disease. International Journal of Molecular Sciences, 24 (5), 4765. https://doi.org/10.3390/ijms24054765
38. Nogaret, P., El Garah, F., & Blanc-Potard, A.-B. (2021). A Novel Infection Protocol in Zebrafish Embryo to Assess Pseudomonas aeruginosa Virulence and Validate Efficacy of a Quorum Sensing Inhibitor In Vivo. Pathogens, 10 (4), 401. https://doi.org/10.3390/pathogens10040401
39. Novoa, B.; T. V. Bowman; L. Zon; A. Figueras. 2009. LPS response and tolerance in the zebrafish (Danio rerio). 26 (2): 326-331.
40. Novoa B.; A. Figueras. 2012. Zebrafish: Model for the study of inflammation and the innate immune response to infectious diseases.
41. Ogino, H., Fujii, M., Ono, M., Maezawa, K., Kizu, J., & Hori, S. (2009). In vivo and in vitro effects of fluoroquinolones on lipopolysaccharide-induced pro-inflammatory cytokine production. Journal of Infection and Chemotherapy, 15 (3), 168-173. https://doi.org/10.1007/s10156-009-0680-1
42. Ogungbemi, A. O.; R. Massei, R. Altenburger, S. Scholz and E. Küster. 2021. Assessing combined effects for mixtures of similar and dissimilar acting neuroactive substances on zebrafish embryo movement. Toxics 9 (5): 104.
43. Opal, S. M.; R. P. Dellinger; J. L. Vincent; H. Masur; D. C. Angus, D. C. 2014. The next generation of sepsis clinical trial designs: what is next after the demise of recombinant human activated protein C? Critical care medicine 42 (7): 1714-1721.
44. Patton, E. E., Zon, L. I., & Langenau, D. M. (2021). Zebrafish disease models in drug discovery: from preclinical modelling to clinical trials. Nature Reviews Drug Discovery, 20, 611-628. https://doi.org/10.1038/s41573-021-00210-8
45. Peng, X., Lin, J., Zhu, Y., Liu, X., Zhang, Y., Ji, Y., Yang, X., Zhang, Y., Guo, N., & Li, Q. (2016). Anxiety-related behavioral responses of pentylenetetrazole-treated zebrafish larvae to light-dark transitions. Pharmacology Biochemistry and Behavior, 145, 55-65.
46. Petersen, K.; M. S. Riddle; J. R. Danko; D. L. Blazes; R. Hayden; et al. 2007. Trauma-related infections in battlefield casualties from Iraq. Ann Surg 245:803-811.
47. Philip, A. M.; Y. Wang; A. Mauro; S. El-Rass; J. C. Marshall; W. L. Lee; A. S. Slutsky; C. C. dos Santos; X.-Y. Wen. 2017. Development of a zebrafish sepsis model for high-throughput drug discovery. Molecular Medicine 23 (1): 134-148. https://doi.org/10.2119/molmed.2016.00188
48. Ramage, A. G., & Villalón, C. M. (2008). 5-hydroxytryptamine and cardiovascular regulation. Trends in pharmacological sciences, 29 (9), 472-481. https://doi.org/10.1016/j.tips.2008.06.009
49. Schlapbach, L. J.; J. Trück; T. Roger. 2020. Editorial: The Immunology of Sepsis-Understanding host susceptibility, pathogenesis of disease, and avenues for future treatment. Frontiers in Immunology 11:1263. https://doi.org/10.3389/fimmu.2020.01263
50. Sisodia, B. S., Kumar, V., Singh, S., Singh, S., Datta, S., & Singh, J. (2020). Zebra Fish Infection Model: From Pathogenesis to Therapeutics. In B. Siddhardha, M. Dyavaiah, & A. Syed (Eds.), Model Organisms for Microbial Pathogenesis, Biofilm Formation and Antimicrobial Drug Discovery (pp. 429-440). Springer. https://doi.org/10.1007/978-981-15-1695-5_22
51. Snitchler, C. L.; D. M. Patel; S. L. Stahlman; A. V. Chauhan; N. Y. Wells; A. A. Mcquistan. 2021. Sepsis hospitalizations among active component service members, US Armed Forces, 2011-2020. MSMR 28 (11): 2-8. https://europepmc.org/article/med/35044733.
52. Staruch, R. M. T.; S. Hettiaratchy. 2019. Warzone trauma and surgical infections. Surgery (Oxford) 37 (1): 58-63.
53. Stein, C.; M. Caccamo; G. Laird; M. Leptin. 2007. Conservation and divergence of gene families encoding components of innate immune response systems in zebrafish. Genome Biology 8 (11): R251.
54. Sullivan, C., Matty, M., Jurczyszak, D., Gabor, K., Millard, P., Tobin, D., & Kim, C. (2017). Infectious disease models in zebrafish. Methods in Cell Biology, 138, 101-136.
55. Sullivan, C., & Kim, C. H. (2008). Zebrafish as a model for infectious disease and immune function. Fish & shellfish immunology, 25 (4), 341-350.
56. Tanaka, T., Mori, M., Sekino, M., Higashijima, U., Takaki, M., Yamashita, Y., Kakiuchi, S., Tashiro, M., Morimoto, K., & Tasaki, O. (2021). Impact of plasma 5-hydroxyindoleacetic acid, a serotonin metabolite, on clinical outcome in septic shock, and its effect on vascular permeability. Scientific reports, 11, 14146. https://doi.org/10.1038/s41598-021-93649-z
57. Torraca, V., & Mostowy, S. (2018). Zebrafish infection: from pathogenesis to cell biology. Trends in cell biology, 28 (2), 143-156. van der Sar, A. M.; B. J. Appelmelk; C. M. J. E. Vandenbroucke-Grauls; W. Bitter. 2004. A star with stripes: zebrafish as an infection model. Trends in microbiology 12 (10): 451-457.
58. Ulloa, L., Brunner, M., Ramos, L., & Deitch, E. A. (2009). Scientific and clinical challenges in sepsis. Current pharmaceutical design, 15 (16), 1918-1935. https://doi.org/10.2174/138161209788453248
59. van der Sar, A. M., Appelmelk, B. J., Vandenbroucke-Grauls, C. M., & Bitter, W. (2004). A star with stripes: zebrafish as an infection model. Trends in microbiology, 12 (10), 451-457.
60. van der Vaart, M.; H. P. Spaink; A. H. Meijer. 2012. Pathogen recognition and activation of the innate immune response in zebrafish. Advances in hematology 2012:159807.
61. Vellinga, N. A., Veenstra, G., Scorcella, C., Koopmans, M., van Roon, E. N., Ince, C., & Boerma, E. C. (2015). Effects of ketanserin on microcirculatory alterations in septic shock: An open-label pilot study. Journal of critical care, 30 (6), 1156-1162. https://doi.org/10.1016/j.jcrc.2015.07.004
62. Wang, Y., Wang, X., Liu, W., & Zhang, L. (2018). Role of the Rho/ROCK signaling pathway in the protective effects of fasudil against acute lung injury in septic rats. Molecular medicine reports, 18 (5), 4486-4498. https://doi.org/10.3892/mmr.2018.9446
63. Weintrob, A. C.; C. K. Murray; X. Jiahong; M. Krauss; W. Bradley; T. E Warkentien; B. A. Lloyd; D. R. Tribble; Infectious Disease Clinical Research Program Trauma Infectious Disease Outcomes Study Group. 2018. Early infections complicating the care of combat casualties from Iraq and Afghanistan. Surgical Infections 19 (3): 286-97. https://doi.org/10.1089/sur.2017.240. White, R.; S. Jeffery; A. Elstone; K. Cutting. 2015. Sepsis and chronic wounds: the extent of the issue and what we should we be aware of. Wounds UK 11 (4): 8-14.
64. Yun, H. C.; J. G. Branstetter; C. K. Murray. 2008. Osteomyelitis in military personnel wounded in Iraq and Afghanistan. Journal of Trauma Injury, Infection, and Critical Care 64 (2): S163-S168.
65. Zhang J, Bi J, Liu S, Pang Q, Zhang R, Wang S, Liu C. 5-HT Drives Mortality in Sepsis Induced by Cecal Ligation and Puncture in Mice. Mediators Inflamm. 2017; 2017:6374283. doi: 10.1155/2017/6374283. Epub 2017 Jun. 13. PMID: 28694565; PMCID: PMC5485362.
66. Zhang, X., Zhao, Y., Wu, Q., Lin, J., Fang, R., Bi, W., Dong, G., Li, J., Zhang, Y., & Cao, J. (2019). Zebrafish and Galleria mellonella: Models to identify the subsequent infection and evaluate the immunological differences in different Klebsiella pneumoniae intestinal colonization strains. Frontiers in Microbiology, 10, 2750.

1. A method of preventing or treating severe inflammation, sepsis, and/or septic shock; comprising a step of administering an effective amount of:
a. a drug that suppresses the activity of 5-hydroxytryptamine (5-HT, serotonin) receptors, to a subject in need of such treatment, wherein the drug optionally comprises one or more of an antagonist, a partial agonist, or an inverse agonist of 5-HT receptors;
b. a drug that inhibits the activity of receptor tyrosine kinases (RTKs), to a subject in need of such treatment, wherein the drug optionally comprises one or more of an inhibitor of platelet-derived growth factor receptors (PDGFRa and PDGFRb), vascular endothelial growth factor receptors (VEGFR1, VEGFR2 and VEGFR3), stem cell factor receptor (KIT), Fms-like tyrosine kinase-3 (FLT3), colony stimulating factor receptor Type 1 (CSF-1R), and/or the glial cell-line derived neurotrophic factor receptor (RET);
c. a dietary supplement comprising a folic acid supplement and/or iron supplement
d. a drug that suppresses the activity of histamine receptors, wherein the drug optionally comprises an antagonist, a partial agonist, or an inverse agonist of histamine receptors, wherein the histamine receptor is H₁, H₂, H₃, or H₄; and/or
e. a drug that increases the level of 5-hydroxytryptamine (5-HT, serotonin) in blood, wherein the drug optionally comprises a selective serotonin reuptake inhibitor (SSRI), serotonin-norepinephrine reuptake inhibitors (SNRI), or monoamine oxidase inhibitor (MAOI).

2. The method of claim 1, wherein the 5-HT receptor is 5-HT_{2A}, 5-HT_{2B}, or 5-HT_{2C}, and optionally 5-HT_{2A}.

3. The method of claim 1, wherein the drug that suppresses the activity of 5-HT receptors is at least one selected from ketanserin, tegaserod, paliperidone, paliperidone palmitate, quetiapine, ziprasidone, brexpiprazole, ritanserin, phenoxybenzamine, cyproheptadine, aripiprazole, chlorpromazine, clozapine, olanzapine, risperidone, nefazodone.

4. (canceled)

5. The method of claim 1, wherein the inhibitor of RTK is sunitinib or sunitinib malate.

6. (canceled)

7. The method of claim 1, wherein the folic acid supplement is levomefolic acid.

8. The method of claim 1, wherein the iron supplement is ferric citrate.

9. (canceled)

10. (canceled)

11. (canceled)

12. (canceled)

13. (canceled)

14. (canceled)

15. The method of claim 1, wherein the drug that suppresses the activity of histamine receptors is at least one selected from mepyramine, triprolidine, cetirizine, cimetidine, ranitidine, tiotidine, thioperamide, iodophenpropit, clobenpropit, tiprolisant, proxyfan, promethazine, and pitolisant, and optionally pitolisant.

16. (canceled)

17. The method of claim 1, wherein the drug that suppresses the activity of histamine receptors is pitolisant.

18. (canceled)

19. (canceled)

20. (canceled)

21. A method of preventing or treating severe inflammation, sepsis, and/or septic shock; comprising a step of administering an effective amount of a composition comprising fenspiride hydrochloride, levomepromazine, folic acid supplement, iron supplement, and/or RTK inhibitors to a subject in need of such treatment.

22. (canceled)

23. (canceled)

24. (canceled)

25. (canceled)

26. (canceled)

27. (canceled)

28. (canceled)

29. The method of claim 1, wherein the SSRI is selected from citalopram, escitalopram, fluvoxamine, fluoxetine, paroxetine, sertraline, and vortioxetine.

30. The method of claim 1, wherein the MAOI is selected from isocarboxazid, phenelzine, selegiline, and tranylcypromine, rasagiline, moclobemide, and iproniazid.

31. The method of claim 1, further comprising co-administering an effective amount of at least one antibiotic,
wherein the severe inflammation, sepsis or septic shock is caused by bacterial infection, and
wherein the drug is one or more selected from ketanserin, tegaserod, brexpiprazole, paliperidone palmitate, and sunitinib.

32. The method of claim 1, further comprising co-administering an effective amount of at least one anti-viral drug,
wherein the severe inflammation is caused by mutation-prone RNA virus infection, and
wherein the drug is one or more selected from ketanserin, tegaserod, brexpiprazole, paliperidone palmitate, and sunitinib.

33. A method of treating severe edema, comprising a step of administering an effective amount a drug,
wherein the severe edema is caused by cardiogenic shock, neurogenic shock, or anaphylactic shock, and
wherein the drug is one or more selected from ketanserin, tegaserod, brexpiprazole, paliperidone palmitate, and sunitinib.

34. (canceled)

35. (canceled)

36. (canceled)

37. (canceled)

38. (canceled)

39. A method of screening for an agent to prevent or treat inflammation, sepsis or circulatory shock using a zebrafish sepsis model, the method comprising subjecting a test agent to zebrafish embryos 3 days post-fertilization (dpf) zebrafish embryos and/or 5-dpf fully developed zebrafish exposed to 20-80 μg/mL or about 60 μg/mL P. aeruginosa LPS in the presence for a predetermined time period 15-30 hours, or about 24 hours, and
detecting vascular leakage (tail fin edema), reactive oxygen species (ROS) production and/or mortality rate of the zebrafish embryos and/or fully developed zebrafish after administering;
identifying an agent as an agent to treat or prevent inflammation, sepsis or circulatory shock if it reduces vascular leakage, ROS and/or mortality compared to untreated zebrafish embryos and/or fully developed zebrafish.

39. (canceled)
